(19) United States
(12) Patent Application Publication
(10) Pub. No.: US 2022/0112270 A1
(43) Pub. Date: Apr. 14, 2022

(54) METHODS OF PRODUCING AND USING RECOMBINANT ALPHA 1-ANTITRYPSIN (AAT) AND COMPOSITIONS THEREOF

(71) Applicant: EXCELLGENE SA, Monthey (CH)

(72) Inventors: Maria João WURM, Montreux (CH); Florian Maria WURM, Montreux (CH); Sabina JANCIAUSKIENE, Hannover (DE); Jürg HAMACHER, Bern (CH); Uz STAMMBERGER, Basel (CH)

(21) Appl. No.: 17/383,109

(22) Filed: Jul. 22, 2021

Related U.S. Application Data
(63) Continuation of application No. PCT/IB20/50581, filed on Jan. 24, 2020.
(60) Provisional application No. 62/796,159, filed on Jan. 24, 2019.

(51) Int. Cl. C07K 14/81; A61P 11/00

(52) U.S. Cl. CPC C07K 14/8125; A61P 11/00

(57) ABSTRACT

Embodiments of the present invention generally relate to recombinant alpha 1-antitrypsin (AAT) proteins, including variants of human AAT with individually introduced mutations, compositions containing such recombinant AAT proteins and carriers, expression plasmids or vectors and host cells that express such recombinant AAT proteins, methods of producing such recombinant AAT proteins, and methods of treating AAT deficiency-related diseases, disorders, and conditions or diseases, disorders, and conditions resulting in protease-induced tissue damage in a subject in need thereof with the recombinant AAT proteins and/or recombinant AAT protein compositions described here. The recombinant AAT proteins derived from mammalian host cells as produced by the methods described here may be produced in large quantities, without any animal components, i.e., highly pure, highly glycosylated, and may be advantageously used over plasma-derived AAT.

### CROSS-REFERENCE TO RELATED APPLICATIONS

This application is a Continuation application relating to and claiming the benefit of commonly-owned, co-pending PCT International Application No. PCT/M2020/050581, filed Jan. 24, 2020, which claims priority to and the benefit of U.S. Provisional Application Ser. No. 62/796,159 filed on Jan. 24, 2019, the contents of each of the foregoing are herein incorporated by reference in its entirety.

### SEQUENCE LISTING

The instant application contains a Sequence Listing, which has been submitted in ASCII format via EFS-Web and is hereby incorporated by reference in its entirety. Said ASCII copy, created on Dec. 29, 2021, is named 178225-010102US-SEQListing.txt and is 39 kilobytes in size.

### FIELD OF INVENTION

The present invention relates generally to recombinant human alpha 1-antitrypsin (AAT) proteins as well as variants thereof, vectors and host cells containing such recombinant AAT, and methods of making and using such AAT products in the treatment of AAT deficiency-related diseases, disorders, and conditions.

### BACKGROUND

Alpha 1-antitrypsin or α1-antitrypsin (AAT, A1AT, A1A) is a broad glycopeptide, an acute phase protein, and an inhibitor of serine proteases, including for example, an inhibitor of the Serine protease inhibitor (Serpin) superfamily that also has anti-inflammatory functions. In humans, AAT is encoded by the SERPINA1 (Serpin Family A Member 1) gene. The wild type mature AAT glycoprotein has 394 amino acids and is N-glycosylated at Asparagine at residue 46 (Asn 46), 83 (Asn 83), and 247 (Asn 247). AAT is also referred to as an Alpha-1 Protease Inhibitor (α1PI) because of its ability to inhibit a variety of proteases, not simply trypsin. It is primarily an inhibitor of neutrophil elastase, but also inhibits cathepsin G, chymotrypsin, plasminogen activator, proteinase 3, and the like (Janciauskiene S. Biochim Biophys Acta, 1535(3):221-235, 2001).

AAT is also characterized as an anti-inflammatory and an immunoregulatory protein in vitro and in vivo (Janciauskiene S, et al. Resp. Med. 105:1129-1139, 2011). The majority of proteins innately fold into their most stable form. However, an essential function of AAT is due to its higher conformational flexibility, which facilitates its binding with a variety of proteases and other substances. AAT is a mainly produced in the liver and released into circulation. At least one of the best recognized functions of this protein is to protect the lungs from damage caused by activated enzymes of inflammatory cells, such as, for example, neutrophil elastase, an enzyme released by a body's white blood cells as they respond to inflammation or infection. To protect connective tissue in the lung from serine protease degradation, a balance between proteases and antiproteases is important and this balance is provided by AAT's antiprotease activity. In the absence of correct quantitative and qualitative amounts of AAT, neutrophil elastase enzymes may disrupt or breakdown elastin, an important protein in connective tissue that enables tissues to return to its original shape after extending or contracting.

An alpha 1-antitrypsin (AAT) deficiency occurs when the body does not make enough of the AAT protein to protect tissues from proteolytic damage, due to genetic defects in the SERPINA1 gene. Many AAT deficiency variants have been described; however, the clinically most significant deficiency is caused by the Z variant (Glutamic Acid to Lysine mutation at position 342; E342K) (Hag I, et al. Am J Respir Cell Mol Biol. 54(1):71-80, 2016).

Severe AAT deficiency in humans is defined by plasma levels of AAT below 11 mmol/L (i.e., about 0.5 g/L). The deficiency of AAT may result from abnormalities in protein stability, secretion, and functional activities. For example, misfolding of the Z-type AAT variant is associated with intracellular accumulation of AAT molecules through ordered self-association (or polymerization). Because the liver is the main organ producing AAT, the retention of the abnormal AAT protein in the liver may result in liver disease (e.g., neonatal cholestasis, liver cirrhosis, liver cancer, etc.). On the other hand, less secretion of defective AAT protein and its extracellular polymerization provide insufficient quantities of AAT to protect the lungs and other organs during acute and chronic inflammatory situations. This condition can result in pulmonary disease (e.g., emphysema, asthma, bronchiectasis, chronic obstructive pulmonary disease (COPD), chronic bronchitis, etc.), and other disorders and conditions, including but not limiting to, for example, panniculitis, Wegener's granulomatosis, and vasculitis.

Preparations of AAT protein purified from human plasma are commonly used in human therapy (Lebing, W. “Alpha-1 Proteinase Inhibitor: The Disease, the Protein and Commercial Production.” In: Bertolini, J, Goss, N., Curling, J. (eds): Wiley and Sons, Inc. (2013) pp. 227-240; Lundblad, R. L. In: Lundblad Biotechnology of Plasma Proteins. Taylor and Francis Group 2013. pp. 285-323). In healthy human blood plasma, AAT is present at a concentration of about 1-2 g/L. Patients with severe inherited AAT deficiency (AATD) are typically treated with human AAT protein derived from human plasma. Severe deficiency is defined by having a known severe deficient genotype/phenotype (e.g., PI*ZZ, PI*Z null, PI* null null, etc.) and/or if an available serum AAT level is less than 0.5 g/L. The patients with severe AAT deficiency generally have an AAT concentration of about 0.1-0.25 g/L (Ferrarotti I, et al. Thorax, 67:669-674, 2012; Stoller J, et al. Ann Am Thorac Soc 12(12):1796-1804, December 2015). One of the most common severe inherited AATDs is PiZZ, arising from a homozygous mutation (Glycine to Lysine at residue 342; Gly342Lys) in the SERPINA1 gene. The PiZZ mutation leads to an intracellular aggregation of the AAT protein that prevents its secretion. A mechanistic link is suggested between intracellular accumulation of a misfolded Z-AAT protein (i.e., gain-of-function or proteotoxicity) and a decreased secretion of Z-AAT protein (i.e., loss-of-function) leading to tissue damage and the eventual development of chronic disease.

Clinically, PiZZ carriers are strongly predisposed to the development of lung emphysema and liver disease. Lung disease, usually in the form of progressive emphysema, develops during the 4th or 5th decade of a human's life, especially in smokers. The PiZZ mutation may result in the development of both an acute, neonatal liver injury and a chronic, progressive liver disease in adulthood. In addition to that, PiZZ carriers display heightened inflammatory or immune responses that make them susceptible to developing panniculitis or granulomatosis with polyangiitis. Several other forms of disease can be present in PiZZ carriers.

Plasma-derived purified preparations of human AAT, as biological products, are used for treating AATD lung disease patients with a purpose of decreasing progression of the disease. Among the group of pathological disorders, other than lung emphysema, that are considered to be linked to reduced levels and/or reduced functional activity of AAT are HIV type 1 infection, hepatitis C infection, diabetes mellitus, fibromyalgia, systemic vasculitis, and necrotizing panniculitis. AAT replacement or substitution therapy for Type-1 diabetes, cystic fibrosis, and graft-versus-host-disease are being evaluated in clinical trials.

However, purified, formulated plasma-derived AAT preparations suffer from several quality-related drawbacks, since these products are contaminated with other proteins, albeit in small quantities. In addition, plasma-derived AAT is derived from pooled blood donations, and thus represent AAT molecules that may not all be identical with respect to their amino acid sequence and glycosylation pattern. More than 100 molecular AAT variants differing from the dominant wild type human AAT have been identified using DNA sequencing technologies. Thus, donors having different mutations in the SERPINA1 gene, but without any clinical presentation of disease, are likely to become a part of the population contributing to the pooled plasma used as a substrate for the generation of purified human plasma AAT products. Because the pooled plasma-derived AAT is from hundreds of donors who have varying genotypes (i.e., not all donors have the MM genotype), there may be a low percentage of mutations that could result in misfolded proteins or have an immunological impact on the patients who receive the pooled plasma-derived AAT from repeated augmentation therapy treatments. Moreover, the isolation of AAT from human plasma is a very complicated and long process, which can modify the naturally circulating plasma AAT protein with respect to its structure, such as by oxidation, inducing AAT polymerization and/or the generation of other chemical forms (that are less inhibitory). The preparations of clinical grade AAT obtained from human plasma by different producers differ in the purification methods, concentrations, and dosage forms, and are expensive.

Numerous approaches for expressing mammalian proteins, including human AAT, in microorganisms have been reported. These include bacteria (EP0137633), yeast (U.S. Pat. Nos. 4,839,283 and 4,752,576; EP0304971), plants (U.S. Pat. No. 6,127,145; Sudarshana M R, et al. Plant Biotechnol J. 4(5):551-9, 2006 September), insects (Chang C J, et al. J Biotechnol. 102(1):61-71, 2003; Morifuji Y, et al. Mol Biotechnol. 60(12):924-934, 2018 (https://doi.org/10.1007/s12033-018-0127-y), mammalian cells (U.S. Pat. Nos. 5,399,684 and 5,736,379; Garver R I, et al. Proc Natl Acad Sci USA 84:1050-1054, 1987), including Chinese hamster ovary (CHO) cells (Paterson T, et al. Appl Microbiol Biotechnol. 40: 691-698, 1994; Lee K J, et al. Glycoconj. J. 30, 537-547, 2013; Amann T, et al. Metab. Eng., 52:143-152, 2019 (epub 2018 Dec. 1: https://doi.org/10.1016/j.ymben.2018.11.014)) and a human neuronal cell line (AGE1.HN®) (Blanchard V, et al. Biotechnol. Bioeng. 108:2118-28, 2011). As an alternative, human recombinant AAT can be produced in the milk of transgenic animals (Archibald A L, et al., Proc. Natl. Acad. Sci. USA 87:5178-5182, July 1990; U.S. Pat. No. 5,650,503; Wright G, et al. Bio/Technology 9:830-834, 1991). Even transgenic silkworms have been recently used to make human AAT (Morifuji Y, et al. Mol. Biotech. 60(12):924-934, 2018).

Recombinant methods based on microbial systems, insect cells, or plant-based technologies resulted in materials that differed in major secondary modifications from human plasma-derived AAT, and thus had either insufficiently complex human-like glycosylations, exposed different non-human-like glycosylations, or lacked them entirely. These features resulted in, among other impacts on functionality, shortened circulation half-lives of such materials when injected into animals, which made them unsuitable for therapy in general.

The generation of transgenic animals, such as sheep or goats, producing the desired proteins in the milk of the females, is a very complex, costly, and long-term exercise in order to raise sufficiently large herds for “industrial milking.” In addition, these methods have drawbacks since certain risks for the contamination of the product by known and unknown pathogenic agents may exist because the animals cannot be raised in virus-, fungus-, and bacteria-free environments. The AAT product needs to be purified from the milk of hundreds of animals, bred, and raised on specialized farms. In spite of major investments by several companies, these attempts were terminated as of decades ago.

Attempts to express recombinant human AAT in cultivated animal cells have also been made, but the overall success has been limited and was deemed insufficient for further development. Both CHO cells and human cell lines have been utilized. In all cases, the amounts of AAT materials obtained were rather low, in spite of major efforts to increase the productivity. In one of the best cases, from a human cell line not widely used in industry, less than 3 g/L was obtained (Ross D, et al. J. Biotech. 162:262-273, 2012). In addition, issues of acceptable quality remained due to the use of fetal bovine serum (FBS) during the production process. FBS, a growth-promoting agent in culture medium, i.e., a medium additive, is typically excluded today from large-scale manufacture processes because of potential risk factors associated with bovine prion diseases. Amann T, et al. (Metab. Eng., 52:143-152, 2019 (epub 2018 Dec. 1: https://doi.org/10.1016/j.ymben.2018.11.014)) discloses the expression of an AAT made in glycoengineered CHO cell lines has been performed, however, again, the yields obtained in an “optimized” Fed-Batch process over 13 days merely reached 150 mg/L (i.e., 0.15 g/L), which again verifies the insufficient productivity to obtain a commercially viable alternative for plasma-derived AAT. Up to 20 liters of such a cell culture process would be necessary to obtain a single 1-week dose of AAT for one patient. There is still a long felt need to produce high yields of AAT (or recombinant AAT used interchangeably here) in view of current technologies utilized by Allende et al. which only yielded 1 g/L of recombinant AAT in CHO cells using an inducible expression system (see, e.g. Lalonde, et al., J Biotechnology 307 (2020) 87-97, 2019, which is incorporated herein by reference). This amount is still too low for commercial viability and occurs at a production rate that is too low as well. Considering that production yields in cell culture do not reflect the yield of a formulated and highly formulated protein, one may assume that less than half of these culture yields would actually be provided to the patient.

Amounts of 100-200 grams of AAT per year are required to supply a single patient requiring AAT replacement therapy. The necessary manufacturing scales of operation for the recombinant product for a future market when using any of the previously described production systems, at cell culture yields of 1-3 g/L, would be extremely large, and thus make a recombinant product extremely costly.

Most importantly, none of the mentioned cell culture-based technologies applied to the synthesis of recombinant human-like AAT have been used to produce material that progressed towards its ultimate use clinically. As a result, patients who require AAT for therapy continue to be dependent on an inconsistent or shrinking supply of product derived from pooled and fractionated human plasma. Thus, there is a need in the art for methods and means for an improved production of glycosylated, therapeutically-effective AAT, and preferably, from a host production system and associated manufacturing technology that has consistently been shown to be an excellent source of therapeutic proteins over several decades.

The known treatment methods vary for AAT deficient patients, but mostly address the symptoms and physiological results of AAT deficiency. These treatments may include a liver/lung transplant, AAT replacement (or also called augmentation) therapy for lung emphysema, and treatment of the symptoms using, for example, bronchodilators, corticosteroids, supplemental oxygen, pulmonary rehabilitation, antibiotics and vaccinations against viral hepatitis and influenza strains, and the reduction or elimination of environmental risk factors. The most widely used treatment of AAT deficiency in patients with pulmonary emphysema is replacement therapy with injectable preparations of plasma-derived AAT. These preparations provide protease inhibition activity to those patients who carry mutant genes responsible for protein variants that provide no or little protease inhibition activity. A certain quantity of protease inhibition is necessary to maintain a balance between proteases and protease inhibitors in certain human tissues, such as the lung. The lack of or insufficient protease inhibition results in tissue-degrading processes, enhanced by inflammations. In addition, in these inflammatory processes, functional levels of AAT provide anti-inflammatory support beyond inhibition of proteases.

The clinically most relevant treatment goal for AAT-related pulmonary disease is to reduce or eliminate the progression of lung damage by augmenting protease inhibition with functional AAT proteins. Replacement or augmentation therapy is a specific therapy for AAT-related diseases and typically uses AAT from the blood plasma of healthy donors in order to increase the AAT protein levels circulating in the blood and other biological fluids of patients with inherited and/or acquired AAT deficiency. The replacement therapy with AAT is generally executed by intravenous infusion of human plasma-derived and purified AAT. In order to achieve a functional AAT level of about 1 g/L to 2 g/L in the blood of these AAT deficient patients, the patient may undergo intravenous infusion. The circulating half-life of endogenous AAT in humans has been shown to be about five days. Endogenous AAT in human plasma has been found to be in a concentration of about 1-1.75 g/L.

To provide a single weekly dose of AAT to an AAT deficient patient, more than half a liter to over 3 liters (e.g., greater than 0.5 L, 1 L, 1.25 L 1.5 L, 1.75 L, 2 L, 2.25 L, 2.5 L, 2.75 L, 3 L, 3.25 L, 3.5 L, 3.75 L, 4 L) of human plasma is needed from a donor. Thus, the presently available supply of plasma-derived AAT is limited and cannot fulfill the increasing use and demand. In spite of numerous attempts over decades to provide a safe and commercially viable source of high-quality AAT preparations by recombinant DNA technologies, none of the commonly known approaches have been satisfactory or considered suitable towards the manufacture of clinically acceptable qualities and quantities of human recombinant AAT for augmentation or replacement therapy.

Augmentation therapy with AAT is not considered to be a cure or a solution for restoring already lost lung function. However, this therapy can reduce the frequency and severity of pulmonary exacerbations and reduce emphysema progression. There are at least four commercially available augmentation therapy products approved by the U.S. Food and Drug Administration (FDA) that are available in the United States. These approved alpha-1 proteinase inhibitor products include Prolastin-C® (Grifols, S.A.), Aralast NP™ (Takeda Pharmaceutical), Zemaira® (CSL Behring LLC), and Glassia® (Kamada Ltd.). However, these are plasma-derived AAT products, which not only are in limited supply, but also may carry a risk of transmitting infectious agents, e.g., viruses, unknown or emerging viruses, and other pathogens. Accordingly, there remains a need for high-yielding, high-quality recombinant alpha 1-antitrypsin products with a reasonably long circulation half-life and bioavailability as a preventive or therapeutic treatment for AAT deficiency that can be administered safely and that are produced in sufficiently large quantities by a process with economic viability.

### SUMMARY

As described here, the present invention and embodiments thereof feature a recombinant AAT protein including variants thereof, compositions, expression vectors, host cells, and efficient methods for manufacturing the AAT protein including variants thereof or encoding the AAT protein including variants thereof, methods for producing the recombinant AAT protein including variants thereof, and methods for treating AAT deficiency-associated diseases, disorders, and conditions, in a subject in need thereof with compositions comprising the recombinant AAT including variants thereof described here. One aspect provides for a recombinant AAT protein, including variants thereof, where the recombinant AAT protein is from any species, including, but not limited to human. The terms “recombinant AAT,” “variants thereof,” or “recombinant AAT variants” are all used interchangeably here, where reference to “human recombinant AAT protein” also includes any of the interchangeable terms, such as for example, variants of human recombinant AAT protein.

An aspect of the invention may be directed to an expression vector and a method of incorporating a nucleic acid fragment containing a nucleotide or nucleic acid sequence which encodes a human AAT protein into an expression vector. Another aspect provides for a mobilizing or helper vector comprising a nucleic acid fragment containing a nucleic acid sequence encoding a transposase, where the transposase is a “cut-and-paste” transposase, such as but not limited to: a piggyBac, Tol-2, Sleeping Beauty, Leap-In, and any other “cut-and-paste” transposase, or the like useful in assisting the transposition of the nucleotide sequence encoding a human AAT protein into at least one host cell.

One aspect may be directed to a method for producing a human recombinant AAT protein including variants thereof, comprising: a) introducing a host cell with an expression vector comprising a nucleic acid fragment containing a nucleic acid sequence which encodes a human recombinant AAT protein, including variants thereof, to isolate a transformant, i.e. a recombinant cell, expressing the human recombinant AAT protein including variants thereof; b) culturing host cells with the transformant, recombinant cells, or expression vector comprising the nucleic acid fragment that comprises a nucleic acid sequence which encodes a recombinant AAT under conditions which allow for the expression of the human recombinant AAT protein; and c) isolating the human recombinant AAT protein, including variants thereof, from the recombinant cells, thereby producing the human recombinant AAT protein including variants thereof. Another aspect may be directed to the nucleic acid fragment comprising a nucleic acid sequence encoding a human recombinant AAT, including variants thereof, that is a CHO-cell codon-optimized sequence.

Another aspect may be directed to the method of producing a recombinant AAT protein including variants thereof, wherein the culturing step comprises: selecting the host cell with the nucleic acid fragment expressing the human AAT protein, wherein the selected cells are clonally-derived cells expressing human recombinant AAT protein. The selecting step comprises: a) growing or culturing the clonally-derived recombinant cells expressing human recombinant AAT protein permanently (constitutively) in a culture medium; b) feeding the clonally-derived cells expressing human recombinant AAT protein with at least one feed; c) maintaining the culture medium at a cell culture temperature sufficient to maintain or promote normal, healthy cells; d) modifying or decreasing the cell culture temperature; e) growing or culturing the clonally-derived cells at the decreased cell culture temperature until the cells express the recombinant AAT protein, for example, human recombinant AAT protein at a titer of about 1 g/L or greater.

A further aspect may be directed to a method for producing a human recombinant AAT protein including variants thereof, comprising: a) introducing into a host cell, e.g., eukaryotic, a first nucleic acid sequence encoding a human AAT protein and at least an additional nucleic acid sequence encoding a transposase; b) culturing the host cell under conditions which allow expression of the first nucleic acid sequence encoding a human AAT protein, including variants thereof, where the additional nucleic acid sequence encoding, for example, a transposase, such as but not limited to for example, piggyBac, may also be in the cell culture and expressed in order to assist in the incorporation of the gene of interest encoding, i.e., for example, a human AAT, where the host cell is transformed with a nucleic acid sequence encoding a human AAT; c) selecting the host cell with the nucleic acid fragment expressing a human AAT protein, wherein the selected cells are clonally-derived cells expressing human recombinant AAT protein; and d) isolating the recombinant AAT protein, including variants thereof, from the host cell or eukaryotic host cell, thereby producing the human recombinant AAT protein, where the step of isolating may comprise purifying the human recombinant AAT protein.

One aspect may be directed to an expression vector, comprising: a nucleic acid fragment containing a nucleotide sequence encoding an AAT protein, where the AAT protein may be, for example, a human AAT protein, including variants thereof, where the nucleic acid fragment is positioned in a multiple cloning site; an intron upstream of the nucleic acid fragment; a mouse or human cytomegalovirus (CMV) promoter upstream of an intron; a 5′ Inverted Terminal Repeat (5′ ITR) upstream of the CMV promoter; a poly-adenosine tail signal sequence downstream of the nucleic acid fragment; an E. coli replication origin sequence downstream of the nucleic acid fragment; a selectable marker sequence downstream of the replication origin sequence; a 3′ Inverted Terminal Repeat (3′ ITR) downstream of the selectable marker sequence, wherein the selectable marker sequence may be distinct from an antibiotic resistance sequence or may alternatively include an antibiotic resistance sequence.

Yet another aspect may be directed to a human recombinant AAT protein including variants thereof, comprising a polypeptide sequence having about 75% or greater identity to, about 80% or greater identity to, about 85% or greater identity to, about 90% or greater identity to, about 95% or greater identity to, about 96% or greater identity to, about 97% or greater identity to, about 98% or greater identity to, about 99% or greater identity to, or about 100% identity to SEQ ID NO: 1.

A further aspect of the invention may be directed to a human recombinant AAT protein including variants thereof, and the methods of producing a human recombinant AAT protein including variants thereof, where the isolated human recombinant protein has a purity of about or greater than about 90%, about or greater than about 95%, about or greater than about 97%, about or greater than about 98%, about or greater than about 99%, about or greater than about 99.9%, or any purity percentage greater than what may be accomplished by the production and isolation and/or purification of a plasma-derived AAT protein.

One aspect of the invention provides a composition, comprising the human recombinant AAT protein including variants thereof produced by any of the methods described herein. A further aspect provides a pharmaceutical composition comprising the human recombinant AAT protein including variants thereof as described here or as produced by the methods described here, where the composition further comprises a pharmaceutically-acceptable carrier, diluent, or medium.

In yet another aspect, a method of treating a subject suffering from an alpha 1-antitrypsin deficiency, comprises administering a human recombinant AAT protein or composition comprising a recombinant AAT protein including variants thereof as disclosed here and a pharmaceutically-acceptable carrier, diluent, or medium, where if the subject is human, then the composition comprises a human recombinant AAT protein including variants thereof, where the AAT deficiency may be ameliorated or improved upon treatment by administering human recombinant AAT protein. Yet another aspect provides a method of treating a subject suffering from any type of protease-induced tissue damage, induced by a variety of underlying diseases, including those that may be caused by inflammation of an undefined origin, where the tissue damage may be ameliorated or improved upon treatment by administering a recombinant AAT protein.

Another aspect provides a method for producing a human recombinant alpha 1-antitrypsin (AAT) protein, comprising: culturing a host cell with a first nucleic acid sequence encoding a human AAT protein and at least a second nucleic acid sequence encoding a transposase, wherein the culturing step occurs at a first period of time at a first temperature and at a second period of time at a second temperature, and optionally at a third period of time at a third temperature.

### BRIEF DESCRIPTION OF FIGURES

The characteristics and advantages of embodiments of the invention will be described in detail in conjunction with the accompanying figures.

FIG. 1A, FIG. 1C, FIG. 1E, and FIG. 1G show the amino acid sequence of the human “wild type” AAT, which is preceded by a leader peptide sequence indicated by bold and underlined text and corresponding nucleic acid sequence with restriction enzyme sites (SpeI and EcoR1) indicated in italicized text (FIG. 1B; FIG. 1D; FIG. 1F; FIG. 1H). FIG. 1A shows the wild type human AAT amino acid sequence without a leader peptide sequence (SEQ ID NO:1). FIG. 1B shows the nucleotide sequence (SEQ ID NO:2) comprising the encoded human AAT protein sequence of SEQ ID NO:1. Various leader peptide signal sequences indicated by bold, underlined text of human AAT amino acid sequences are in FIG. 1C (SEQ ID NO:3) with a natural human AAT leader sequence (SEQ ID NO:11) and FIG. 1D, a nucleotide sequence (SEQ ID NO:4) comprising the encoded human AAT protein sequence with natural human AAT leader peptide sequence of SEQ ID NO:3, FIG. 1E (SEQ ID NO:5) with a human IgG heavy chain leader sequence (SEQ ID NO:12) and FIG. 1F, a nucleotide sequence (SEQ ID NO:6) comprising the encoded human AAT protein sequence with human IgG heavy chain leader peptide sequence of SEQ ID NO:5, and FIG. 1G (SEQ ID NO: 7) with a Chimpanzee AAT leader sequence (SEQ ID NO:13) and FIG. 1H, a nucleotide sequence (SEQ ID NO:8) comprising the encoded human AAT protein sequence with Chimpanzee AAT leader sequence of SEQ ID NO:7 are presented as examples of leader sequences from different sources.

FIG. 2A, FIG. 2B, FIG. 2C, and FIG. 2D show the 6504 single-stranded nucleic acid sequence of the plasmid pXLG6-AAT (distributed over 4 individual figures, but with a continuous accounting for the nucleotide sequence; SEQ ID NO:9), comprising the sequence encoding a CHO-cell codon-optimized AAT protein sequence that is capitalized and underlined (positions 1948 . . . 3214, and the restriction enzyme fragment encoding the AAT protein sequence is inserted in the pXLG6 plasmid vector at restriction enzyme recognition sites SpeI to EcoR1, where the italicized text represents the restriction enzyme recognition sites (positions 1948 . . . 1953; positions 3209 . . . 3214, respectively) in the direction of the 5′ end to the 3′ end.

FIG. 3 shows a plasmid map of the 5268 base pair pXLG6 vector used for expressing DNA of interest, typically inserted downstream of the EF-1-alpha intron element (998 . . . 1941 bp). The plasmid map also includes the ITR piggyBac terminal repeat sequences (321 . . . 13 bp/4050 . . . 4299 bp) and mammalian resistance marker for puromycin (Puro-r; 3834 . . . 3235 bp), as well as the bacterial ampicillin resistance marker (Amp-r; 5251 . . . 4391 bp).

FIG. 4A shows a plasmid map of the pXLG5 vector used as the “mobilizing” expression vector in co-transfections, where the gene coding for the Piggy Bac transposase enzyme (mPBase) (FIG. 4B) indicates the position of the transposase gene (905 . . . 2686 bp) which is driven by the cytomegalovirus (CMV) promoter (209 . . . 863 bp). This vector also contains a Zeocin resistance marker, including its own promoter (Zeo; 3870 . . . 4244 bp). FIG. 4B shows the PiggyBac amino acid sequence encoded by a sequence within the pXLG5 vector (mPBase 905-2686) (SEQ ID NO: 10).

FIG. 5 demonstrates the batch (filled circles) and fed-batch (filled triangles) culture performance of the clonally derived cell population CHO-rAAT_c112. The kinetics of cell growth in viable cell densities/mL (Top) (VCD; 10⁶ cells/mL) and the cell culture viabilities (%) (Bottom) over 10 days or 14 days are shown, respectively.

FIG. 6 shows that the 14-day fed-batch cell culture process of 2 clonal cell lines (No. 112 (light grey) and No. 423 (dark grey)) with respect to cell growth (VCD; cells/mL) (Top) and productivity kinetics (AAT titer; mg/L) (Bottom).

FIG. 7 shows a comparison of the average of AAT titers (mg/L) produced using different media conditions under the conditions described in FIG. 9 as measured on Days 12 and 14 (n=4).

FIG. 8 shows the analysis of the production of recombinant AAT from five different clonal AAT-producing cell lines (CHO-AAT Nos. 112, 275, 423, 555, 585) in batch processes (Day 6) and fed-batch processes (Day 14). The CDM columns (checkered) indicate a Day-6 culture using a commercially available chemically-defined medium (CDM; HyClone™ CDM4CHO; GE Healthcare), the XLG columns (Grey) indicate Day-6 batch cultures with chemically-defined medium, XLG_E21_07 (ExcellGene S.A.), and the XLG Fed-Batch (XLG FB) columns (Black) indicate Day-14 Fed-Batch process cultures with both a chemically-defined medium, XLG_E21_07 (ExcellGene S.A.) and a chemically-defined feed (Feed A; ExcellGene S.A.).

FIG. 9 shows an example of an experiment applying high-throughput culture conditions executed with the goal to obtain a high-yielding fed-batch process using the cell line CHO-rAAT_c112 to produce recombinant human AAT while using as a basic production medium, the chemically-defined medium XLG_E21_07 (ExcellGene S.A.).

FIG. 10 shows the profile of recombinant AAT purified from the AAT-producing CHO cells as analyzed through size exclusion chromatography. The purified recombinant AAT protein was collected from the main peak that eluted at about 12 minutes to about 14 minutes.

FIG. 11A-FIG. 11D show that recombinant AAT derived from the recombinant CHO cells efficiently reduces elastase activity in vitro and effectively forms a complex with its target-elastase. More specifically, FIG. 11A shows that elastase converts a substrate (N-succinyl-Ala-Ala-Ala-p-nitroanilide) over time to generate spectrophotometrically measurable absorbance (filled black circles) in duplicate. In the presence of recombinant or plasma-derived AAT, this conversion is inhibited to a significant degree (open circles, recombinant AAT; black triangles, plasma-derived AAT). FIG. 11B represents the data of FIG. 11A in the form of a column diagram, indicating that in both cases of the presence of AAT elastase activity is reduced by about 50%.

FIG. 11C shows the inhibitory activity of rec. AAT or Prolastin (GRIFOLS) towards elastase in a different way as shown in A and B and done independently from the AB experiment. Briefly, 50 ng of elastase was incubated with various concentrations of rec.AAT or Prolastin for 30 minutes at room temperature in 50 mM Tris, 1M NaCl, 0.05% (w/v) Brij35, pH 7.5. Upon addition of the neutrophil elastase substrate (MEOSUC Ala Ala Pro Val AMC, Bachem, Catalog #11270, 100 mM) for human neutrophil and porcine pancreatic elastase assays, fluorescence emission was recorded at 460 nm (380 nm excitation), in kinetic mode for 10 minutes at 37° C. Change in Relative Fluorescence Unit (RFU) was measured for each AAT concentration by subtracting RFU from blank for each point. The results were plotted using Prism 5.0 (GraphPad, San Diego, Calif.). FIG. 11D shows an image of an SDS PAGE gel (7.5% polyacrylamide) in which plasma-derived and recombinant AATs, respectively, are run alone or after incubation with elastase, showing an interaction between the two molecules resulting in a higher molecular weight band for the complex of AAT with elastase. Marker; Lane 1: 5 μg plasma-derived AAT (pAAT) (Zemaira) alone; Lane 2: 5 μg pAAT with 2.35 m elastase (pAAT+elastase); Lane 3: 5 μg recombinant AAT (recAAT); Lane 4: 5 μg recAAT with 2.35 m elastase (recAAT+elastase); Lane 5: 2.35 m elastase alone.

FIG. 12 shows that recombinant AATs (recAAT; ExcellGene; R&D Systems) and plasma-derived AAT (Prolastin), as well as AAT containing mouse plasma inhibit the potent protease trypsin over a range of AAT concentrations from the low nanogram/ml range to over 4 μg/ml (i.e., 4,000 ng/ml). The data are presented as an average of triplicate experiments and the horizontal lines near the symbols indicate the variations from the mean value.

FIG. 13 shows that increasing amounts of recombinant AAT (0 mg/ml-1 mg/ml) reduces endotoxin (1 μg/ml) (or lipopolysaccharide (LPS))-induced TNF-alpha (TNF-α) release (pg/ml)).

FIG. 14A and FIG. 14B show that recombinant AAT (1 mg/ml) inhibits endotoxin (1 μg/ml) (LPS)-induced expression of TNF-α (Top; FIG. 14A) and IL-6 (Bottom; FIG. 14B), respectively, in human adherent peripheral blood mononuclear cells (PBMCs) as compared to the hypoxanthine guanine phosphoribosyl transferase (HPRT) housekeeping gene. Each bar represents two independent repeats.

FIG. 15A-FIG. 15D show that recombinant AAT, as well as plasma-purified AAT can be efficiently transported across several layers of skin, in an in vitro model of human skin (epiCS®; CellSystems). The epiCS® skin model was used for in vitro toxicology and efficacy testing of AAT in skin penetration, where positive staining for AAT was demonstrated (darker stained areas indicated by arrows) in FIG. 15 B as opposed to control without AAT in FIG. 15 A. FIG. 15 C shows epiCS® culture supernatant analysis by Western blot which demonstrated time-dependent positivity for AAT in epiCS® skin treated with both recombinant AAT (recAAT) and plasma AAT (pAAT), but not in controls without AAT (Co). FIG. 15 D shows that skin irritation as identified by total levels of IL-18 (pg/ml), a pro-inflammatory cytokine and marker of skin irritation, was inhibited by both recAAT (10 mg; light grey) and pAAT (10 mg; dark grey) at 6 hours as compared to control which did not have any AAT present (0 mg AAT control; black).

### DETAILED DESCRIPTION

Detailed embodiments of the present invention are disclosed here; however, it is to be understood that the disclosed embodiments are merely illustrative of the invention that may be embodied in various forms. In addition, each of the examples given in connection with the various embodiments of the invention is intended to be illustrative, and not restrictive. For example, the methods of producing a recombinant human alpha 1-antitrypsin may also apply to producing any other desired recombinant protein. A person of skill in the art would understand based on the teachings here and what is known in the art, how to prepare an expression vector which encodes the desired protein for introduction into an appropriate host cell or host cell population, culturing the host cell under conditions which allow for expression of the desired recombinant protein, and isolation of the desired recombinant protein, for further use in applications, including but not limited to therapeutic, research, and diagnostic.

All terms used here are intended to have their ordinary meaning in the art unless otherwise provided. All concentrations are in terms of percentage by weight of the specified component relative to the entire weight of the topical composition, unless otherwise defined.

As used here, “a” or “an” shall mean one or more. As used here, when used in conjunction with the word “comprising,” the words “a” or “an” may mean one or more than one. As used here “another” means at least a second or more.

As used here, all ranges of numeric values include the endpoints and all possible values disclosed between the disclosed values. The exact values of all half-integral numeric values are also contemplated as specifically disclosed and as limits for all subsets of the disclosed range. For example, a range of from 0.1% to 3% specifically discloses a percentage of 0.1%, 1%, 1.5%, 2.0%, 2.5%, and 3%, and all intervening percentages. Additionally, a range of 0.1% to 3% includes subsets of the original range including, for example, from 0.5% to 2.5%, from 1% to 3%, from 0.1% to 2.5%, etc. It will be understood that the sum of all weight % of individual components will not exceed 100%, unless otherwise noted.

It is understood that aspects and embodiments of the invention described here include “comprising,” “consisting,” and “consisting essentially of” aspects and embodiments.

Reference to “about” a value or parameter herein includes (and describes) embodiments that are directed to that value or parameter per se. For example, description referring to “about X” includes description of “X.” Numeric ranges are inclusive of the numbers defining the range.

The present disclosure provides an alternative supply of human alpha 1-antitrypsin (AAT) including variants, such as, for example, molecular amino acid variants, of human AAT that have individually-introduced mutations, generated by genetic engineering of mammalian host cells, resulting in an abundant and reproducible supply of the AAT protein. Embodiments of the disclosure are not necessarily comprehensive but provide to a person of ordinary skill in the art sufficient insight to follow the methods to generate or produce high-level protein expression from high-yielding cells, such as but not limited to, Chinese hamster ovary (CHO) cells, to obtain recombinant AAT (rAAT or recAAT) including variants thereof, for example, recombinant human AAT. In embodiments of the invention, the subject suffering from an AAT deficiency may include any human or alternatively any animal, where an animal may be classified as a mammal, including humans, domestic and farm animals, and zoo, sports, or pet animals, such as dogs, horses, cats, cattle, etc. Preferably, the mammal is human. Other embodiments of the invention are directed to recombinant AAT protein, including variants thereof, which are for the same species as those AAT-deficient subjects who are suffering and/or to be treated.

Embodiments show that a reliable, large-scale supply of high-quality AAT can be provided by recombinant mammalian cells in bioreactors that are optimized for high-yield productivity. The recombinant CHO cell lines or modified CHO cells, adapted to suspension culture, described here provide a basis for large-scale manufacturing, since the high-yielding small- to mid-scale processes developed here are scalable to about 1,000 L and 10,000 L operations. Thus, the production of hundreds of kilograms of modified CHO cell-derived recombinant human AAT including variants thereof can become a reality; whereas, in past decades of research and development, the supply of such recombinant human AAT in large quantities and of high quality and purity have failed, possibly also for commercial reasons that did not provide a balance of cost in manufacturing and market economics.

The problem of a low yield and substandard quality of recombinant AAT product, and methods of producing the same is solved by embodiments of the present invention, for example those embodiments that provide methods for generating highly productive, clonally-derived cell lines from a fast-growing host cell and its derivatives, i.e., optimized and thus modified CHO cells adapted for suspension culture, and the use of cell line development approaches and process conditions using very rich animal component-free media and/or feed supplements. Together, these methods may generate human AAT-producing cell cultures resulting in volumetric yields of recombinant AAT in fed-batch processes in the multi-gram/liter range, e.g., up to and including about 10 g/L or greater. In addition, but not limited to a specific characteristic of the produced recombinant AAT protein, it is possible to modulate the culture medium and certain process conditions in such a way to generate recombinant human AAT including variants thereof with a high level of terminal sialic acids and other secondary modifications that alter the glycosylation pattern and status of such CHO-derived AAT. Glycosylation is useful for protecting proteins such as AAT from removal from the circulating blood stream which is one factor of enhancing the circulation half-life of recombinant AAT in subjects, thereby extending the half-life of recombinant AAT in AAT-deficient subjects treated with recombinant AAT including variants thereof, where the subject is human in one embodiment.

Embodiments of the disclosure are directed to the materials and methods for generating or producing recombinant AAT preparations including variants thereof useful for treating subjects or patients suffering from an AAT-deficiency by, for example, substitution or augmentation therapy, or that could be used for a number of other diseases are disclosed where AAT may not be processed correctly or may be prevented from circulating at all, or at a reduced half-life, in an AAT-deficient subject. The AAT including variants thereof produced by the materials and methods described here may include a recombinant AAT that is glycosylated, with a high level of sialic acids, for example, terminal sialic acids, allowing for an enhanced circulation half-life of AAT in patients or subjects suffering from an AAT deficiency who have been treated by augmentation or substitution therapy. Because of the biological activities of AAT and increased demands for AAT products, there has been a long desired and active interest in obtaining or producing high quality recombinant AAT including variants thereof, and in large quantities, for human therapeutic use. The methods disclosed here allow for the production of extremely high purity recombinant AAT including variants thereof made in modified Chinese hamster ovary cells cultivated in a bioreactor, where large scale production may occur, and the use of the produced recombinant AAT including variants thereof in the treatment of human disease or conditions where AAT is deficient or absent.

### Human Alpha 1-Antitrypsin (AAT) Sequence

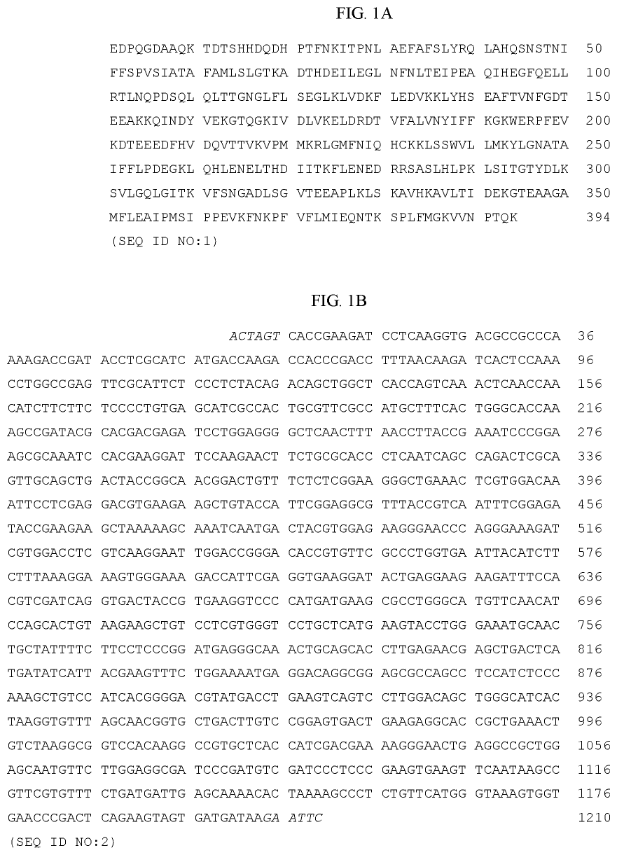

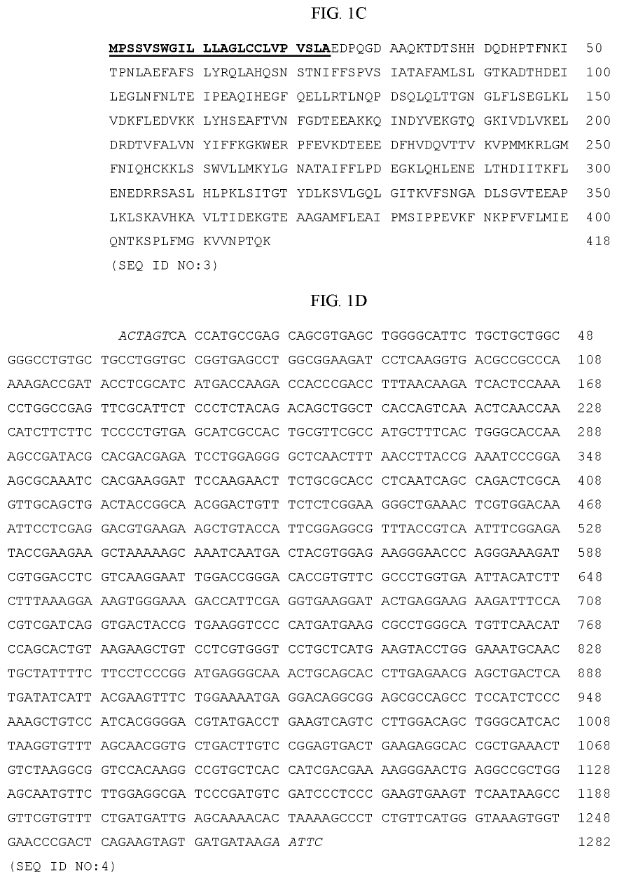

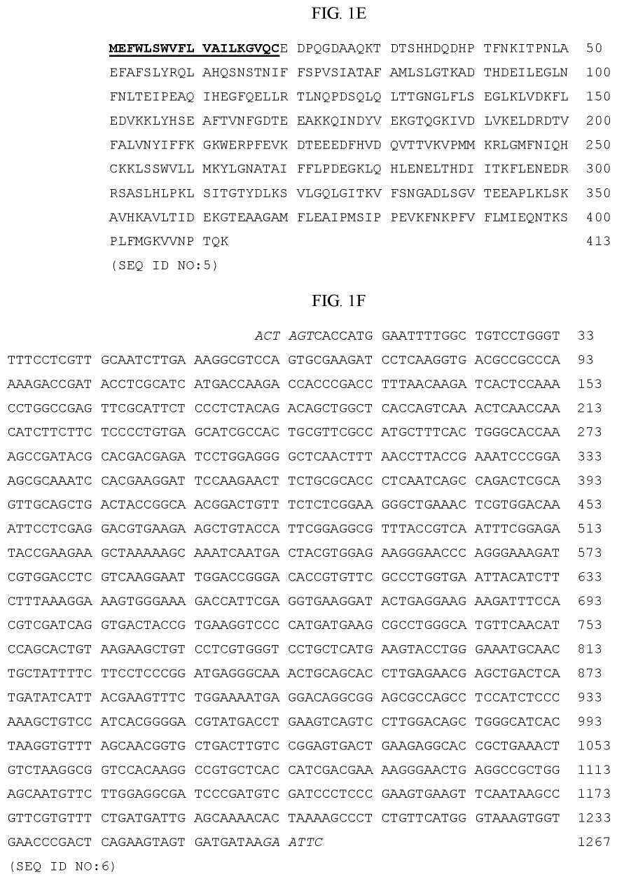

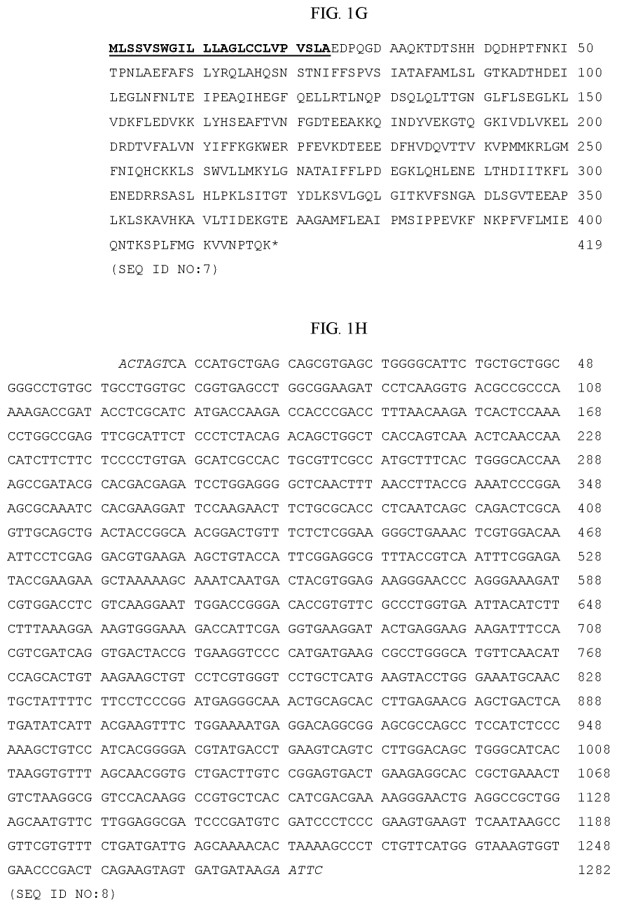

In one embodiment, the AAT sequence of interest is identical to the sequence published in Long G L, et al. (Biochemistry 23(21):4828-4837, 1984). It represents the M-allele, i.e., the most common and functional human Alpha 1-Antitrypsin. The full-length wild type AAT amino acid sequence comprises 394 amino acids corresponding to the human M-allele (FIG. 1A; SEQ ID NO:1). However, a leader sequence (i.e., secretory signal peptide sequence) of the wild type human AAT sequence may be replaced with a different leader sequence, such as but not limited to, the leader sequence of a human heavy chain IgG1 sequence, a human serum albumin leader sequence, a Chimpanzee AAT leader sequence, a mouse Ig Kappa light chain leader sequence, and others. TABLE 1 provides alternative leader sequences which may be incorporated into the AAT sequence. Another leader sequence is the “natural” AAT leader sequence (SEQ ID NO: 10) which also cleaves the leader peptide sequence from the mature AAT. The presence and location of signal peptide cleavage may be predicted and compared for the “natural” AAT leader sequence as well as the other leader sequences by the SignalP 4.1 program (H. Nielsen. Methods Mol. Biol. 1611:59-73, 2017. doi: 10.1007/978-1-4939-7015-5_6). One embodiment provides for a nucleic acid fragment comprising a nucleic acid sequence (SEQ ID NO:4) containing an AAT amino acid sequence comprising the natural AAT leader sequence (SEQ ID NO:3), where an expression vector comprising this nucleic acid fragment may be introduced into a host cell in order to produce human recombinant AAT vector using the methods described here.

TABLE 1

| SIGNAL PEPTIDE SEQUENCES | PROTEIN | SPECIES |
|---|---|---|
| MPSSVSWGILLLAGLCCLVPVSLA | Natural AAT | H. sapiens & Hylobates sp. |
| (SEQ ID NO: 11) | | |
| MEFWLSWVFLVAILKGVQC | IgG Heavy chain | H. sapiens |
| (SEQ ID NO: 12) | | |
| MLSSVSWGILLLAGLCCLVPVSLA | AAT | Pan troglodytes |
| (SEQ ID NO: 13) | | (Chimpanzee) |
| MKWVTFISLLFLFSSAYS | Serum albumin | H. sapiens |
| (SEQ ID NO: 14) | | |
| MTRLTVLALLAGLLASSRA | Azurocidin | H. sapiens |
| (SEQ ID NO: 15) | | |
| METPAQLLFLLLLWLPVSDTTG | Ig kappa Light | H. sapiens |
| (SEQ ID NO: 16) | chain | |
| METDTLLLWVLLLWVPGSTG | Ig kappa Light | Mouse & Hamster |
| (SEQ ID NO: 17) | chain | |

The leader sequence or signal sequence may be cleaved off before secretion from the cells. Including a natural human AAT leader peptide sequence of 24 amino acids, the AAT amino acid sequence may comprise 418 amino acids. (See, FIG. 1B; SEQ ID NO:3). Alternatively, other leader peptide sequences of varying lengths may be included in the AAT protein sequence. In one embodiment, the leader sequence has at least about 10 residues, at least about 15 residues, at least about 19 residues, or at least about 24 residues. Another embodiment may be directed to a leader sequence that is from the same species as the desired AAT, for example, human leader sequence and human AAT. A further embodiment may be directed to a leader sequence that is cleaved before secretion.

It is understood that for a protein to maintain its correct conformation, glycosylation is a useful factor. The absence of glycans or glycosylation may result in rapid clearance from circulation, a decreased half-life, decreased stability, and/or misfolding of the protein, which may result in aggregation or may more easily aggregate or degrade, thereby resulting in a loss of activity, while any of these factors may affect the stability of the protein. In fact, plasma-derived AAT disadvantageously contains a highly diverse glycan signature, i.e., since the plasma-derived AAT is collected from pooled blood, that is likely to be based on AAT polypeptides with non-identical sequences. The plasma-derived AAT molecules may reflect both the genetic and physiological factors (e.g., aging or disease, etc.) of an individual from whom the plasma is collected. Whereas the glycosylation pattern of recombinant AAT may be engineered to be more uniform since there is no individual variability with respect of the polypeptide sequence backbone of all AATs produced by the recombinant host cell.

In one embodiment, a recombinant alpha 1-antitrypsin (AAT) protein including variants thereof is an active, glycosylated protein that is about 90% to about 100% free of contaminants, such as but not limited to, non-human components, animal components, or human components which induce an undesirable immune response. The recombinant AAT protein, which includes variants thereof, described herein may have a purity of about 90% to about 100%, about 95% to about 99.9%, or about 98% to about 99%; a purity of greater than about 95%, greater than about 98%, greater than about 99%, or greater than about 99.9%; or a purity of about 95%, of about 98%, about 99%, about 99.9%, or about 100%.

Another embodiment may be directed to an active recombinant AAT protein including variants thereof described here that comprises a protease inhibition activity equivalent to or greater than the protease inhibition activity of a plasma-derived AAT protein, where the protease is, for example, elastase. In one embodiment, the elastase inhibition activity of the recombinant AAT (recAAT) protein including variants thereof may be about the same as that of a plasma-derived AAT protein. Another embodiment may be directed to the elastase inhibition activity of recAAT including variants thereof that may be greater than about 0% to about 20%, greater than about 5% to about 25%, or greater than about 10% to about 30% of the elastase inhibition activity of a plasma-derived AAT protein.

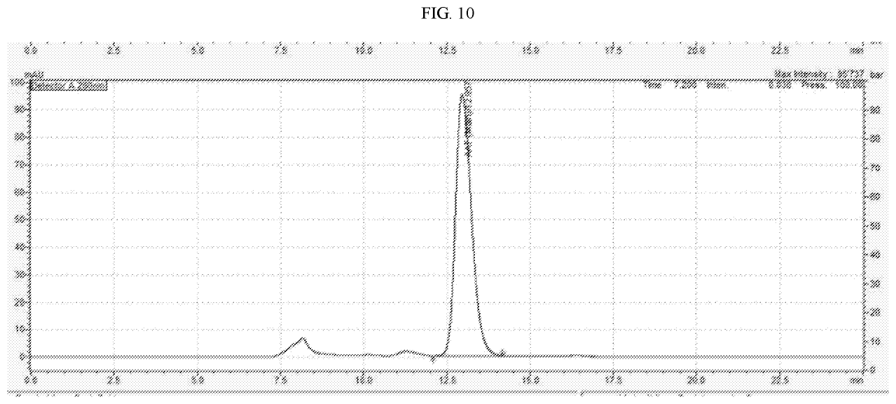

A further embodiment may be directed to the relationship between elastase and alpha 1-antitrypsin, where the recombinant AAT described here inhibits elastase activity. FIG. 10A shows that the absorbance at 405 nm increases the longer an elastase substrate is exposed to this enzyme. In the presence of both plasma-derived and recombinant AAT, added in the same amount to the reaction, the increase in absorbance is reduced in rate by about 50%. This data is expressed in a bar graph (FIG. 10B), indicating the same inhibition rate for elastase by the two AATs, i.e., recombinant AAT and plasma-derived AAT. FIG. 10C demonstrates that both plasma-derived AAT (pAAT) and recombinant AAT (recAAT) form a complex with the elastase in the reaction mixture, indicated by a higher molecular weight band in Lanes 2 and 4 as compared to AAT without elastase in Lanes 1 (pAAT) and 3 (recAAT), when the reaction mixes are subjected to SDS-PAGE.

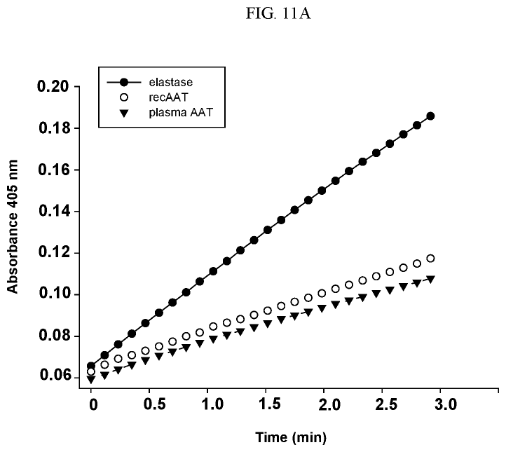

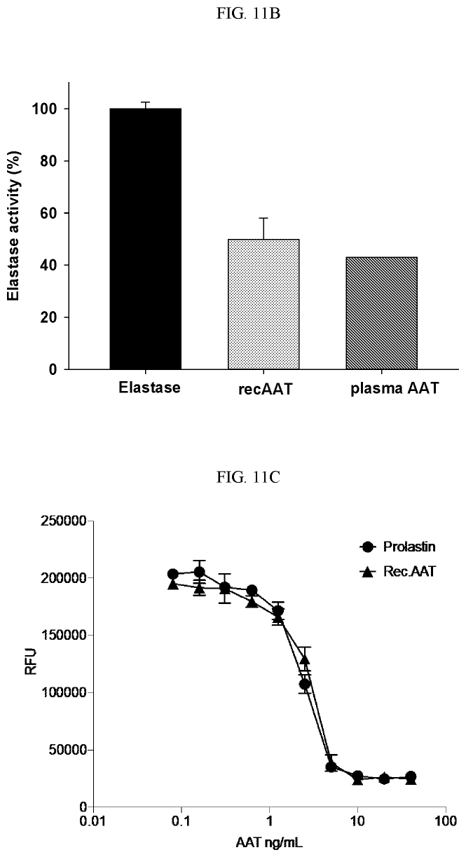

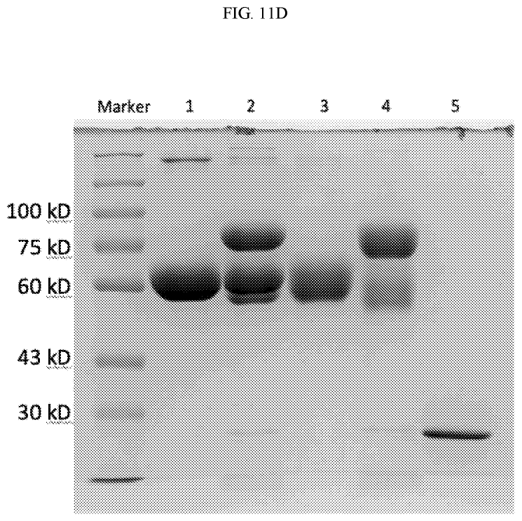

A further embodiment may be directed to the relationship between trypsin and alpha 1-antitrypsin, where the recombinant AAT, or variants thereof, described here inhibits trypsin activity in the same or similar way as human plasma-derived AAT, or crude mouse plasma containing AAT, or another recombinant AAT (R&D systems), derived from a human neuronal cell line. FIG. 11 shows inhibitory activity of AATs of different origins with trypsin, another potent protease as an example. A fluorogenic peptide Mca-RPKVE-Nval-WRK(Dnp)-NH₂ (SEQ ID NO:18) was used as a substrate for trypsin (Mca: (7-Methoxycoumarin-4-yl)acetyl; Nval: Norvaline; Dnp: 2,4-Dinitrophenyl), applying different concentrations of the aforementioned AATs from 0 ng/ml to about 4,000 ng/ml. The mouse serum contained 1.2 mg/ml of AAT.

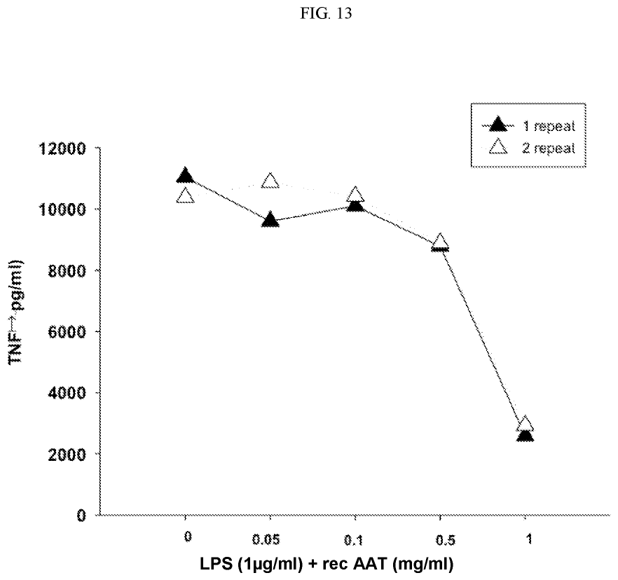

AAT deficiency may be associated with an inflammatory condition. Accordingly, in a further embodiment, the active recombinant AAT protein or variant thereof described here, may have high and broad-ranging anti-inflammatory activities, where the anti-inflammatory activity may be higher than that of a plasma-derived AAT protein. AAT may prevent or reduce the negative effects of TNF-alpha, for example, TNF-alpha-induced apoptosis, by, for example, blocking the TNF-alpha receptor. FIG. 13 shows that mRNAs in peripheral blood cells for TNF-alpha (FIG. 13A) and for IL-6 (FIG. 13B) are reduced when these cells are incubated with a given amount of LPS together with recombinant AAT, but not in the absence of recombinant AAT. Embodiments of the invention may be directed to an oxidized recombinant AAT including variants thereof that may block the effects of a TNF-alpha response. The recombinant AAT protein including variants thereof may, in some embodiments, decrease an inflammatory response by about 10% to about 100%, about 15% to about 90%, about 20% to about 80%, about 13%, about 18%, about 80%, about 81%, about 82%, about 83%, about 84%, about 85%, or greater than about 10%, greater than about 11%, greater than about 12%, greater than about 13%, greater than about 14%, greater than about 15%, greater than about 16%, greater than about 17%, greater than about 18%, greater than about 19%, greater than about 20%, greater than about 50%, or greater than about 80%.

A further embodiment may provide for a human recombinant AAT protein comprising a polypeptide sequence having a SEQ ID NO: 1 mutation, where the mutation is at least one of: a phenylalanine to a leucine at position 51 (F51L), a methionine to valine mutation at position 351 (M351V), or a methionine to valine mutation at position 358 (M358V). Also contemplated are other mutations that would replace amino acid residues which would normally be oxidized or result in a reduced ability to inhibit neutrophil elastase. In one embodiment, any methionine of SEQ ID NO: 1 that could be oxidized would instead have a mutation from a methionine to, for example, a valine. Alternatively, the methionines may be mutated to isoleucines or a combination of valines and isoleucines. In certain embodiments, the at least one mutation may be selected from at least one amino acid residue of: cysteines, tryptophans, phenylalanines, tyrosines, and histidines, where the mutation of one of these residues may prevent the oxidation at that particular position. A further embodiment may provide for at least one mutation that produces thermal stability. In certain embodiments, the at least one mutation may be selected from at least one amino acid residue of: phenylalanine, asparagine, threonine, arginine, and histidine residues, where the mutation of one of these residues may result in a thermal stability greater than without the mutation. For example, the phenylalanine may be mutated to leucine, asparagine to serine, threonine to isoleucine, or any other amino acid that has greater thermal stability that the initial amino acid.

Yet another embodiment may be directed to a recombinant AAT protein including variants thereof, such as for example, human recombinant AAT, comprising about 3 moles of sialic acid or greater per mole of AAT, about 4 moles of sialic acid or greater per mole of AAT, about 5 moles of sialic acid or greater per mole of AAT, about 6 moles of sialic acid or greater per mole of AAT, about 7 moles of sialic acid or greater per mole of AAT, about 8 moles of sialic acid or greater per mole of AAT, about 10 moles of sialic acid or greater per mole of AAT, or about 12 moles or sialic acid or greater per mole of AAT. A further embodiment may be directed to a recombinant AAT protein including variants thereof comprising about 3 moles of sialic acid per mole of AAT to about 12 moles of sialic acid per mole of AAT, about 3 moles of sialic acid per mole of AAT to about 10 moles of sialic acid per mole of AAT, about 5 moles of sialic acid per mole of AAT to about 8 moles of sialic acid per mole of AAT, about 4 moles of sialic acid per mole of AAT to about 6 moles of sialic acid per mole of AAT, and the like. Another embodiment may be directed to a recombinant AAT protein including variants thereof comprising greater than 3.5 moles of sialic acid per mole of AAT or about 5.5 moles of sialic acid or greater per mole of AAT. Yet a further embodiment may be directed to a recombinant AAT protein including variants thereof comprising at least about the same moles of sialic acid per mole of AAT as that of a plasma-derived AAT, or a recombinant AAT protein including variants thereof with a sialic acid content of at least about 10% to about 80% greater than or exceeding that of plasma-derived AAT. In a further embodiment, the sialic acid content exceeds that of a plasma-derived AAT protein by at least about 10%, by at least about 15%, by at least about 20%, or by less than about 90%, by less than about 85%, by less than about 80%. It is understood that the sialic acid molecules available on recombinant AAT including variants thereof stabilizes the protein and may extend the half-life of the protein which is particularly useful in maintaining levels of recombinant AAT protein in the circulation of a subject suffering from an AAT deficiency.

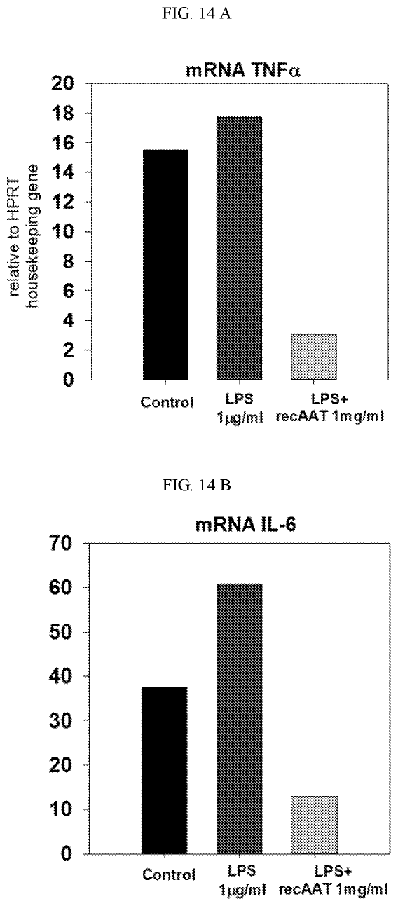

A further embodiment may be directed to recombinant AAT preparations including variants thereof that may pass several layers of human tissue, such as skin or other organ separating materials, such as but not limited to, alveoli, bronchi, bronchioles, pleura, and the like. FIG. 14A and FIG. 14B show results of an experiment in which small quantities of an AAT preparation were applied onto the “external” surface of an artificial skin model. After incubation, AAT was identified below the skin layers of this in vitro system for human skin using a Western Blot approach which detected AAT via rabbit polyclonal anti-human AAT antibody. Without being bound by theory, the recombinant AAT, including variants thereof, may be delivered to sites where there is a deficiency in AAT or misfolded AAT by traversing the external layers of skin, through the epidermis, dermis-epidermis junction, dermis, or some or all layers of skin. The recombinant AAT may be administered in a manner sufficient to deliver the recombinant AAT to locations including but not limited to the skin, or layers thereof, alveoli, bronchi, bronchioles, pleura, capillaries, blood vessels, arteries, and the like.

As long as a recombinant AAT protein maintains its activity of inhibiting protease activity, any sequence of the amino acid sequence shown in SEQ ID NO: 1 may be modified by at least one of substitution, insertion, or deletion of the partial amino acid for use. For example, the modified sequence may include an amino acid sequence having an amino acid sequence identity of about 70% or greater, about 80% or greater, about 90% or greater, about 95% or greater, about 98% or greater, or about 99% or greater, compared to the amino acid sequences of SEQ ID NO:

In one embodiment, the nucleotide sequence of the AAT protein may be a nucleotide sequence of any desired species, such as for example, human AAT protein, or a modified sequenced obtained by optimizing the nucleotide sequence to be suitable for expression in host cells. Another embodiment may provide a nucleotide sequence encoding an AAT protein having a sequence of at least one of the amino acid sequences selected from the sequences of SEQ ID NO:1, SEQ ID NO:3, SEQ ID NO:5, and SEQ ID NO:7, or an amino acid sequence identity of about 70% or greater, about 80% or greater, about 90% or greater, about 95% or greater, about 98% or greater, or about 99% or greater, compared to at least one of the amino acid sequences selected from the sequences of SEQ ID NO:1, SEQ ID NO:3, SEQ ID NO:5, and SEQ ID NO:7. Specifically, the nucleotide sequence can be any one selected from the sequences of SEQ ID NO:2, SEQ ID NO:4, SEQ ID NO:6, and SEQ ID NO:8, or a nucleotide sequence having a substantially similar sequence homology. A substantially similar sequence homology means that any nucleotide sequence that may have a nucleotide sequence identity of about 40% or greater, about 50% or greater, about 60% or greater, about 70% or greater, about 80% or greater, about 90% or greater, about 95% or greater, about 98% or greater, or about 99% or greater compared to by sequence alignment of at least a nucleotide sequence selected from SEQ ID NO:2, SEQ ID NO:4, SEQ ID NO:6, and SEQ ID NO:8.

### Host Cell Expression System

For more than three decades, Chinese hamster ovary (CHO) cells have been used for large scale manufacturing of pharmaceutically relevant proteins, based on suspension cultivated cells in bioreactors. CHO cells are very diverse in their phenotypic potential due to their origin as immortalized cells that are constantly evolving and have been shown to be adaptable to very different modes for growth and production (Wurm F. M., Processes, 1:296-311, 2013; Wurm F. M., Nat Biotechnol. 22(11):1393-1398, 2004; Wurm F. M., and Wurm M. J., Processes, 5(2):20, 2017). In one embodiment, the mammalian cells that are useful in the methods of producing recombinant AAT include a potent recombinant cell line for expression and scale-up in bioreactors derived from a non-recombinant host cell line with selected phenotypes for manufacturing, such as for example, modified CHO cells (CHOExpress™ cells; ExcellGene SA). This non-recombinant host cell line has phenotypic features of exceptionally high growth rate, a high maximal cell density under batch and fed-batch culture with certain media formulations, and when transfected with suitable vectors, recombinant progeny inherits these phenotypes with high fidelity. When using transfection appropriate expression vectors (e.g., vectors that drive the expression of the gene of interest (GOI) permanently-constitutive expression vectors) and appropriate selection and clonally-derived cell populations, the derived cell lines have a high synthetic capacity, and a high viability under fed-batch cultures, during which recombinant product formation will occur. In another embodiment, the mammalian recombinant cells are fast-growing, high yielding (>5 g/L with many protein targets), robust at high densities (>20 million cells/mL, up to 50 million cells/ml in Fed-batch processes), with a very high sub cultivation ratio (>1/30), ranging from subcultivation ratio of 1 to 2 to 1 to 100, and have the highest synthetic capacity, and the ability to maintain high viability (>90%) over an extended number of days, for example, 7 days, 11 days, 14 days, 17 days, greater than 7 days, greater than 11 days, greater than 14 days, etc. A further embodiment is directed to mammalian recombinant cells that are modified Chinese hamster ovary (CHO) cells having these features, including, e.g., CHO-rAAT, or such as but not limited to clonally derived cell populations such as, CHO-rAAT_c112, CHO-rAAT_c423, and the like. The recombinant CHO cells described here may rapidly grow (under 20 hours/cell doubling) through the described culture program, and more specifically, for growth in animal component-free media or in chemically defined media. These recombinant CHO cells have been generated from a non-recombinant host cell line CHOExpress™ cells—grown for 3 decades in animal-component free media —, that can be traced back to an initial CHO cell line obtained from an academic laboratory (Puck T T, et al. J Exp Med., 108(6):945-56, 1958).

In one embodiment, the compositions and formulations of the media, i.e., the production medium and the feed media used for culturing the mammalian host cells from which the recombinant AAT including variants thereof is derived, are known by their name and concentration of each component, such that certain components of the media can be modified in concentration or can be removed entirely. Also, the addition of certain components can be done without negatively impacting the overall performance of the medium, but by enhancing the productivity and/or the quality of the desired AAT. These modifications may, accordingly, influence the secondary modifications of the AAT molecules produced by these cells during a fed-batch process.

### Expression Vector System for Transfection and Selection of Recombinant Cell Populations

Another embodiment may provide a nucleic acid construct comprising a nucleic acid fragment containing one or more nucleotide sequences encoding the desired AAT protein, including variants thereof, where the desired AAT protein may include a human AAT protein or any variants thereof. The construct may comprise an expression vector into which a sequence has been inserted, such as in a cassette. The expression vector may include the coding sequence for an AAT protein, including variants thereof and a human AAT protein. For example, an expression vector comprising a nucleic acid sequence encoding a human AAT protein and a selectable marker sequence, where both are positioned in opposite reading frames and in between a 5′ Inverted Terminal Repeat (5′ ITR) and a 3′ Inverted Terminal Repeat (3′ ITR) may be useful for transforming host cells in order to produce human recombinant AAT protein, where the selectable marker sequence comprises a puromycin resistance gene sequence. A further embodiment may provide for an expression vector comprising a nucleic acid fragment containing a nucleotide sequence encoding a human AAT polypeptide sequence having about 70% or greater identity to, about 75% or greater identity to, about 80% or greater identity to, about 85% or greater identity to, about 90% or greater identity to, about 95% or greater identity to at least one sequence of: SEQ ID NO:1, SEQ ID NO:3, SEQ ID NO:5, or SEQ ID NO:7. Yet another embodiment may provide for a nucleic acid fragment containing at least one of a nucleotide sequence encoding a human AAT polypeptide sequence, where the nucleotide sequence has about 70% or greater identity to, about 75% or greater identity to, about 80% or greater identity to, about 85% or greater identity to, about 90% or greater identity to, about 95% or greater identity to at least one sequence of: SEQ ID NO:2, SEQ ID NO:4, SEQ ID NO:6, or SEQ ID NO:8.

In one embodiment, an expression vector may be used to transfer a nucleic acid sequence encoding the desired protein, for example, but not limited to an AAT protein or a human AAT protein, or variants thereof, into at least one host cell (in vitro). Expression vectors may be equipped with a nucleic acid sequence encoding a selectable marker, restriction enzyme sites, appropriate control elements, such as promoter and termination sequences, among other components. The expression vector may also comprise regulatory sequences, including, but not limited to, non-coding sequences, such as, for example, introns and control elements, i.e., promoter and terminator elements or 5′ and/or 3′ untranslated regions, that may be useful for expressing the coding sequence in host cells. Suitable vectors and promoters are known to those of ordinary skill in the art, many of which may be commercially available.

Non-limiting examples of suitable promoters may include constitutive promoters and inducible promoters, such as for example, a CMV promoter, an SV40 early promoter, an HSV promoter, an EF-1 a promoter, an actin promoter, and the like. Briefly, for expression purposes, a host cell may recognize a promoter sequence, where the promoter sequence is a DNA sequence. The promoter may be operably linked to a DNA sequence encoding a protein of interest, such as for example, an AAT protein or a human AAT protein, or variants thereof. The promoter may be positioned with respect to an initiation codon of the DNA sequence encoding the desired AAT protein in the expression vector in a manner such that the promoter may drive transcription or translation of the nucleic acid sequence encoding the AAT protein. The promoter sequence may contain transcription and translation control sequence which mediate the expression of the AAT protein.

An appropriate selective marker will generally depend on the host cell, and appropriate markers for different hosts are well known in the art. Such selectable markers may confer to transformants the ability to utilize a metabolite that is usually not metabolized by the host cell. A selectable marker may confer the ability of transformants to grow in the presence of an antibiotic, such as for example, puromycin, where the selectable marker is a puromycin resistant gene (Puro-r). The selectable marker coding sequence may be cloned into a suitable plasmid using methods generally employed in the art. Examples of suitable plasmids include pXLG5 or pXLG6. Conventional techniques of molecular biology, recombinant DNA, immunology, and the like are within the skill of the art. After nucleic acid sequences that encode the AAT protein, or other protein of interest, including variants thereof, have been cloned into the construct or expression vector, the construct or expression vector may be used to transform at least one host cell in order to express an AAT recombinant protein, such as for example, a human AAT recombinant protein, or variants thereof. The host cell that may be transformed for the purpose of expressing an AAT protein according to the embodiments described here may be chosen from a wide variety of host cells. The various examples of expression vector components and host cells presented here are not meant to limit their scope but may be employed in practicing the aspects and embodiments presented here.

Another embodiment may provide an expression vector, comprising: a nucleic acid fragment containing a nucleotide sequence encoding a human AAT protein, wherein the nucleic acid fragment is positioned in a multiple cloning site; an intron upstream of the nucleic acid fragment; a cytomegalovirus (CMV) promoter upstream of an intron; a 5′ Inverted Terminal Repeat (5′ ITR) upstream of the CMV promoter; a poly-adenosine tail signal sequence downstream of the nucleic acid fragment; a replication origin sequence downstream of the nucleic acid fragment; a selectable marker sequence downstream of the replication origin sequence; and a 3′ Inverted Terminal Repeat (3′ ITR) downstream of the selectable marker sequence. The selectable marker sequence may comprise in one embodiment, for example, a nucleic acid sequence of a puromycin resistance gene, where a person of skill in the art would understand how to select an appropriate selectable marker sequence and use its related counterpart, i.e., the antibiotic, such as puromycin, in order to select the clonally-derived cells containing the gene of interest, e.g., human AAT, from the cell culture. Moreover, the person of skill in the art would also understand to position the nucleic acid fragment with the gene of interest and the selectable marker sequence in opposite reading frames and between the 5′ inverted terminal repeat (ITR) and the 3′ ITR.

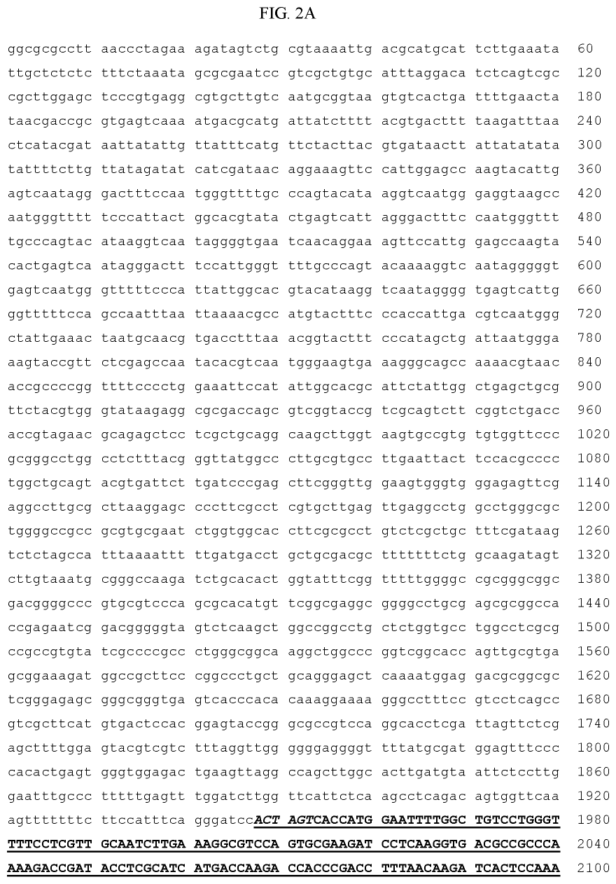

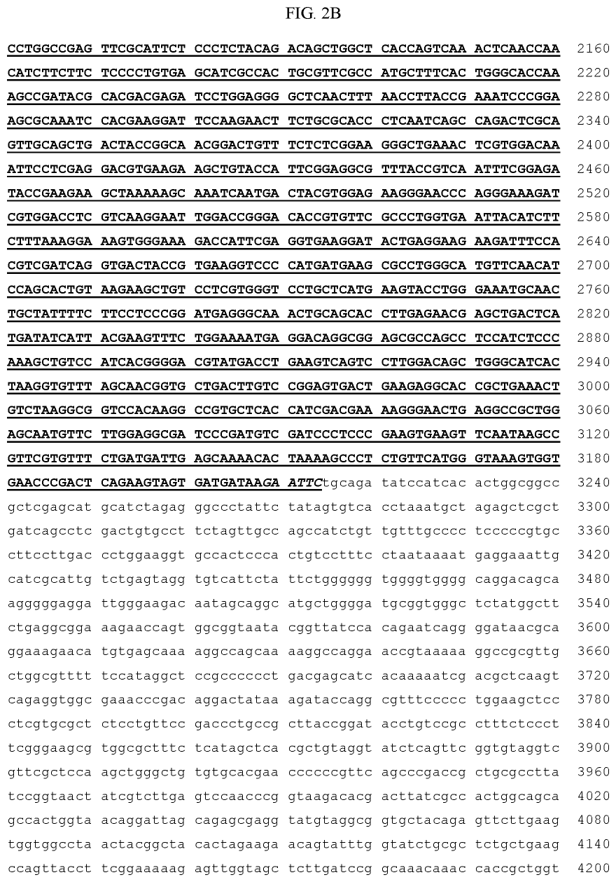

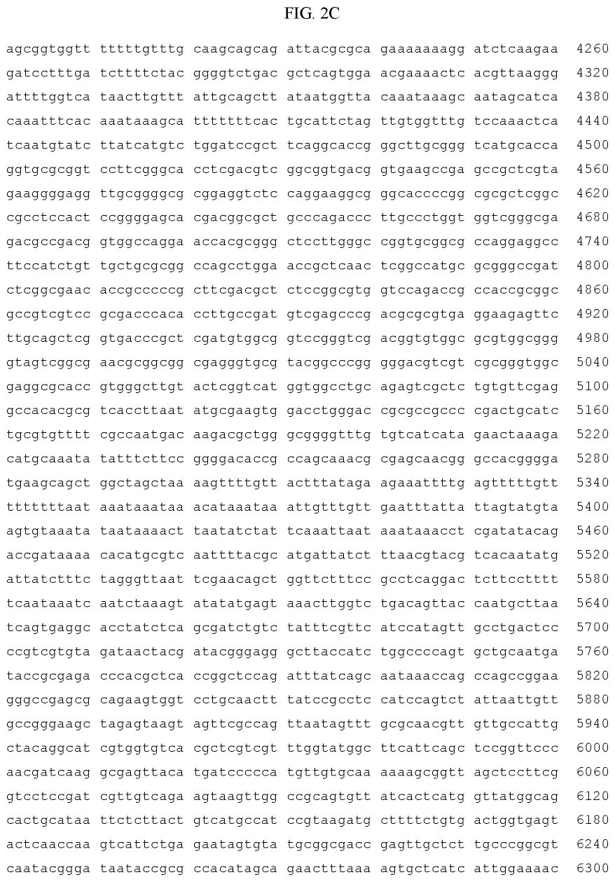

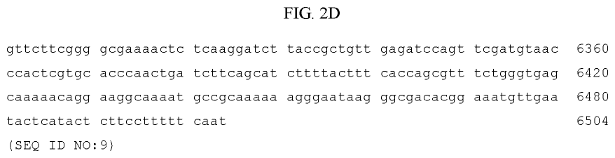

Another embodiment of the expression vector may be directed to the nucleic acid fragment containing a cDNA sequence encoding a human AAT protein. Non-limiting selectable marker sequences may include an antibiotic resistance sequence, a thymidine kinase sensitive to ganciclovir selection, triclosan resistance sequence, a metabolic selection sequence, such as for example, a sequence comprising a dihydrofolate reductase gene or a glutamine synthetase gene, and the like, or combinations thereof. In one embodiment, the selectable marker sequence and/or antibiotic resistant gene is a puromycin resistance gene, an ampicillin resistance gene, a zeocin resistance gene, a geneticin resistance gene, a gene for any other desired selectable marker, such as for example, dihydrofolate reductase or glutamine synthetase, and the like, or combinations thereof. Another embodiment may be directed to an expression vector where the nucleic acid fragment and the selectable marker sequence are positioned in opposite reading frames and in between the 5′ ITR and the 3′ ITR. A further embodiment of the expression vector provides a nucleotide sequence encoding a human AAT polypeptide sequence of any one or more of SEQ ID NO: 1, SEQ ID NO:3, SEQ ID NO:5, and SEQ ID NO:7 (FIG. 1A; FIG. 1C; FIG. 1E; FIG. 1G, respectively) or any other human AAT protein sequence, where the nucleotide sequence may be selected from at least one of SEQ ID NO:2, SEQ ID NO:4, SEQ ID NO:6, and SEQ ID NO:8 (FIG. 1B; FIG. 1D; FIG. 1F; FIG. 1H, respectively). In another embodiment, the expression vector comprises a nucleotide sequence having about 40% or greater identity to, about 50% or greater identity to, about 60% or greater identity to, about 70% or greater identity to, about 75% or greater identity to, about 80% or greater identity to, about 85% or greater identity to, about 90% or greater identity to, about 95% or greater identity, or about 99% or greater identity to at least one of SEQ ID NO: 9 (FIG. 2A-FIG. 2D), SEQ ID NO:2, SEQ ID NO:4, SEQ ID NO:6, SEQ ID NO:8, or the nucleotide sequence comprises at least one of SEQ ID NO: 2, SEQ ID NO:4, SEQ ID NO:6, SEQ ID NO:8, and SEQ ID NO:9.

In yet a further embodiment, a co-transfection system comprising a donor vector expressing the gene of interest (GOI), i.e., the AAT protein including variants thereof, and a mobilizing vector expressing the gene of a transposase that recognizes Inverted Terminal Repeats (ITRs) framing the sequence of the GOI, may be used to efficiently incorporate the gene of interest, for example, the nucleic acid sequence of AAT into at least one host cell for producing recombinant AAT protein including variants thereof. As would be understood by a person of skill in the art, the method of co-transfecting a donor vector comprising any gene of interest as well as the other methods disclosed here may be utilized in order to produce a recombinant protein encoded by the associated gene of interest. A further embodiment of the method of producing a human recombinant AAT protein, including variants thereof, may be directed to the step of introducing comprising co-transfecting the host cell with a vector containing the first nucleic acid sequence encoding a human AAT protein and with a vector containing the additional nucleic acid sequence encoding a transposase. An additional embodiment of the method may provide a helper vector or expression vector comprising a nucleic acid sequence or helper mRNA encoding a transposase that is introduced into the host cell or cell line with a vector or expression vector containing a nucleic acid sequence encoding a human AAT protein, where the nucleic acid sequence comprising the gene of interest (e.g., AAT protein, human AAT protein, etc.) integrates into the genome of the host cell and not the nucleic acid sequence from a mobilizing or helper vector (e.g., use of mRNA transposase in transfections without the use of any plasmid containing the DNA for transposase).

Briefly, a transposon is a genetic element that allows for efficient transposition between vectors and chromosomes by a “cut-and-paste” mechanism. Since the transposase expressed by the mobilizing or helper vector recognizes the transposon-specific ITRs of the donor vector containing the gene of interest, the transposase may “cut” the donor vector at the ITRs and then “paste” the donor vector sequence comprising the gene of interest and selectable marker sequence into the chromosomal DNA of the host cell, for example into TTAA chromosomal sites. Advantage of the transposon, for example, the piggyBac, system is that the sequence size for transposition is essentially unrestricted, it is non-viral, and highly efficient. Non-limiting examples of a transposase useful in embodiments of the invention include: piggyBac, Tol-2, Sleeping Beauty, Leap-In, and any other “cut-and-paste” transposases, or the like. Expression vectors or helper vectors comprising a transposase may include pD2500 vectors, particularly for the Leap-In transposase (ATUM℠; https://www.atum.bio/products/expression-vectors/mammalian#3; Newark, Calif.) or the vectors for the Tol-2- or Sleeping Beauty-based transfections (Balasubramanian S. Thesis No. 6563 (2015) “Study of Transposon-Mediated Cell Pool and Cell Line Generation in CHO Cells,” Swiss Federal Institute of Technology (EPFL), Lausanne, Switzerland).

In yet a further embodiment, the expression vector that is introduced into cells and its subsequent expression of the GOI may contain additional sequences, such as CHO-cell derived endogenous retroviral sequences, that may facilitate the integration of such expression vectors into the active chromatin of the non-recombinant CHOExpress™ host cell line. Such endogenous retroviral sequences may belong to the family of A-type retroviral sequences (Anderson K, et al. Virology 64, 5, 2021-2032, 1990), the family of C-type sequences (Anderson K, et al. Dev. Biol. Stand. 75:123-132, 1991), or any other family of repetitive sequences in eukaryotic genomes that are clustered preferentially in active regions of the genome (Mager D L and Stoye J P. 2014. Microbiol. Spectr. 3(1):MDNA3-0009-2014). These additional endogenous retroviral DNA sequences may either represent a full-length (non-functional) retroviral sequence or shorter fragments thereof. This approach may mediate homologous recombination events in cells that result in integration of the GOI sequences into a region of the genome that contains an endogenous retroviral DNA sequence (Wurm F M, et al. “Retrotargeting: Use of defective retroviral DNA fragments to improve recombinant protein production in mammalian cells.” Animal Cell Technology: Products for Today, Prospects for Tomorrow, edited by R. E. Spier et al., Butterworth-Heinemann Ltd., 1994, pp. 24-29).

In yet another embodiment, the expression vector for the GOI interest may be constructed for high-level productivity from recombinant cells by combining a transposon-based gene transfer approach with a homologous recombination approach for integration into active chromatin of the genome of the DNA receiving cells. Non-limiting approaches for transfecting cells and selecting recombinant cell populations include those that use targeted gene transfers into cells via zinc (Zn)-finger nucleases (Bibikova M, et al. Science. 300(5620):764, 2003), single chain homing nucleases (Grizot S, et al. Nucleic Acids Research. 37(16):5405-5419, 2009), or CRISPR/Cas 9 processes (Jinek M, et al. Science 337(6096):816-821, 2012).

Another embodiment of the disclosure is directed to expression vector constructs used to obtain high-level AAT expression from transfected mammalian cells. (FIGS. 2, 3 and 4). The plasmid vector pXLG6-AAT comprises a nucleic acid sequence that encodes the complete human AAT protein sequence described here, including the corresponding leader sequence, which is inserted in the multi-cloning site (MC S) of the pXLG6 plasmid vector. Also encompassed in an embodiment of the invention is a mobilizing or helper vector, i.e., the plasmid pXLG 5 comprising a transposase, such as but not limited to, a PiggyBac transposase (mPBase), or other transposases including, but not limited to Tol-2, Sleeping Beauty, Leap-In, any other “cut-and-paste” transposase, and optimized versions thereof. The pXLG 5 is co-transfected with pXLG6-AAT in mammalian host cells, such as for example, modified CHO cells (e.g., CHOExpress™ cells; ExcellGene S.A.) or the like, where the modification allows for cells to grow to a high cell density, for example, but not limited to more than 20 million cells/mL, a high sub-cultivation ratio of, such as but not limited to more than 1/20, and recombinant CHO cell process yields having expression levels of over 1 g/L, over 3 g/L, over 5 g/L, over 6 g/L, or over 7 g/L, among other advantages. The co-transfection into mammalian host cells may occur in varying amounts where the transposase expression vector generally has a low molar ratio relative to the GOI (e.g., the AAT expressing vector described here), as the transposase expression vector is a mobilizing or helper vector which aids in the integration or incorporation of the GOI into the genome of the host cells, while the transposase nucleic acid sequence avoids integration. Non-limiting weight/weight ratios of the transposase vector to the GOI vector may include, but are not limited to, about 1:1, about 1:3, about 1:9, about 1:10, about 0.75:9.25, about 0.5:9.5, about 0.25:9.75, about 0.1:9.9, less than about 1:10, less than about 1:9, less than about 1:3, less than about 0.75:9.25, less than about 0.5:9.5, less than about 0.25:9.75, less than about 0.1:9.9, greater than about 0.1:9.9, greater than about 0.25:9.75, greater than about 0.5:9.5, greater than about 0.75:9.25, greater than about 0.9:9.1, greater than about 1:9, greater than about 1:10, about 1:10 to about 0.1:9.9, about 0.75:9.25 to about 0.25:9.75, about 0.5:9.5 as well as any intervening or additional ratios that allow for successful incorporation of the GOI into the host cell genome. Since the transposase vector does not contain ITRs, the transposase would not integrate into the host cell genome, would however over time be eliminated from cells by degradative intracellular processes and thus, the transposase vector is merely active in this co-transfection protocol for a short time (transiently) to assist with the incorporation or integration of the GOI (e.g., AAT expression cassette) into the host cell genome. The same principle applies when Transposase-encoding mRNA is used in a co-transfection with GOI-encoding expression vector DNA.

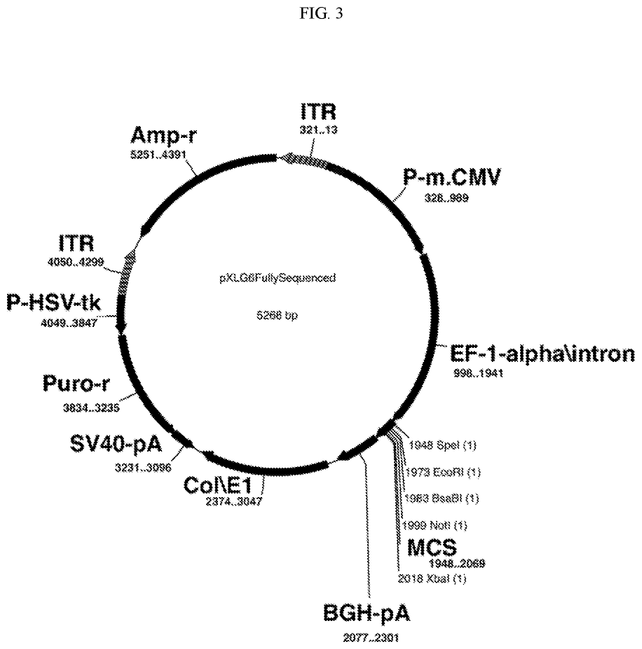

In one embodiment, the pXLG 6 expression vector cassette for the gene of interest (GOI) may be contain a strong constitutive promoter/enhancer derived from a mouse Cytomegalovirus (mCMV) sequence and other useful elements, such as splice-donor sequences, and another expression cassette for the constitutive expression of a selective marker (e.g., the gene encoding for puromycin resistance (Puro-r): PAC—puromycin N-acetyl-transferase) driven by a Herpes Simplex Thymidine Kinase promoter (HSV TK). The GOI sequence, such as for example, human AAT sequence of the disclosure (see, FIG. 1B; FIG. 1D; FIG. 1E; FIG. 1H), may be cloned into the multi-cloning site (MCS) of pXLG 6 (see, FIG. 3). Moreover, in another embodiment, the GOI sequence may comprise a human AAT sequence directed to an allele other than the M-allele.

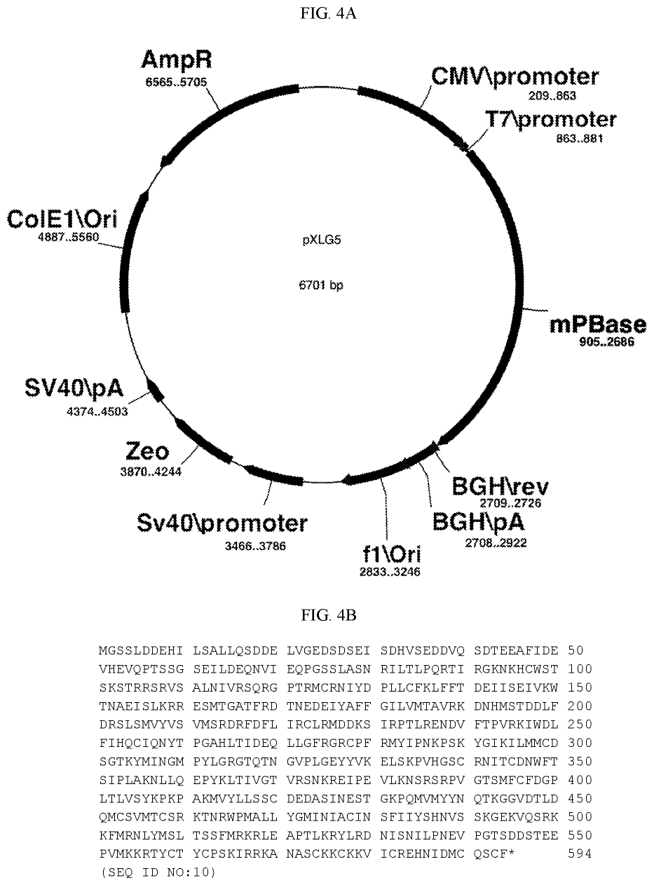

These two expression cassettes may be framed by inverted terminal repeat sequences, 5′ ITR and 3′ ITR. These two ITRs or other ITRs are recognized by a transposase protein, including those but not limited to the PiggyBac transposon of the cabbage looper moth (Trichoplusia ni), the Tol-2 transposon (Kawakami K. Genome Biol. 8(Suppl 1):S7, 2007) or the Sleeping Beauty transposon (Aronovich E L, et al. Human Molecular Genetics, 20:R14-R20, 2011), the Leap-In transposase (ATUM℠; https://www.atum.bio/products/expression-vectors/mammalian#3; Newark, Calif.), or any other DNA mobilizing system using transposases. In order to introduce the transposase protein to host cells, a second vector that expresses, e.g., a PiggyBac transposase or a Sleeping Beauty transposase, may be co-transfected with the plasmid vector comprising the gene of interest. One embodiment relates to the second vector, pXLG 5, comprising a PiggyBac transposase. (See, FIG. 4A). The pXLG 5 plasmid vector contains a corresponding expression cassette encoding the PiggyBac transposase (mPBase). In an embodiment of the disclosure, the ratio of plasmid DNA in co-transfections of CHO cells of GOI vector (e.g., pXLG6-AAT vector) and transposase vector (pXLG5-mPBase vector or helper vector) is 9:1 (by weight). Thus, 90% of the transfection cocktail contains the GOI vector. Higher and lower ratios of helper vector to GOI vector are not excluded and may include ratios, such as but not limited to weight percentages of about 1% of helper vector to about 99% of GOI vector, 5% helper vector to about 95% of GOI vector, about 15% helper vector to about 85% GOI vector, 20% of helper vector to 80% of GOI vector, and the like, such that there is a successful co-transfection that results in the production of active, mature recombinant AAT including variants thereof.

Upon transfection with a chemical transfection reagent (CHO4Tx® kit; ExcellGene SA) and following an optimized procedure, hundreds, if not thousands of plasmids—here a mixture of two different nucleic acid vectors—may be transferred into the nucleus of the host cells. The transposase vector may drive the transcription of the transposase gene and the synthesis of the transposase. The transposase proteins may then recognize the ITR sequences in the GOI vector and excise them from the plasmid. Subsequently, the excised GOI cassette may be integrated into the genome of the host cell mediated by the transposase (Matasci M, et al. Biotechnol Bioeng. 108(9):2141-50, 2011 Apr. 25 Epub). Another embodiment is directed to modified host CHO cells with at least one GOI cassette encoding a human AAT protein including variants thereof. In a further embodiment, about 5-30 copies, about 5-15 copies, or 10-20 copies, of the GOI cassettes may be integrated into the genome of the cloned recombinant CHO cell lines derived from such transfections. Since the PiggyBac transposase has a preference for integration into active chromatin, the expression levels of such recombinant cell lines are found to be high.

### Transfection and Selection

Another embodiment of the invention may be directed to the transfection and selection of recombinant human AAT expressing cells. Antibiotic resistance selection of the modified CHO cells co-transfected with the donor plasmid vector comprising the gene for AAT protein and the mobilizing plasmid vector comprising a transposase gene, such as but not limited to the PiggyBac transposase gene, suggests that the surviving cells have both the GOI and the antibiotic resistance gene, such as for example, a puromycin resistance gene integrated and expressed, or any other resistance providing DNA that is in the plasmid vector and has transformed the host cells, while the transposase gene of the mobilizing vector is not transformed in the host cells. Both the transfection and the selection may occur with cells that grow rapidly without any aggregation under suspension culture in cell culture media and are not at any time exposed to animal component-derived substances. The selection pressure, during about 7 days to about 10 days after transfection, may be maintained under very stringent conditions. These conditions include replacing the medium containing the selection-providing agent every day. Once the cells rapidly grow again and cell viability has been re-established to high values, the cultures may be sub-cultivated using typical and commonly used techniques. This heterogenous population of cells, once further grown and expanded in the absence of any antibiotic selective agent, is considered therefore recombinant and expresses the human AAT protein at very high levels, such as but not limited to about 1 g/L to about 10 g/L, greater than about 2 g/L, greater than about 5 g/L, greater than about 6 g/L, greater than about 8 g/L, greater than about 9 g/L, about 2 g/L, about 3 g/L, about 4 g/L, about 5 g/L, about 6 g/L, about 7 g/L, about 8 g/L, about 9 g/L, about 10 g/L, or any intervening amounts. Thus, an embodiment of the disclosure is directed to recombinant cells expressing high levels of active, highly glycosylated human AAT and clonal cell lines thereof.

In one embodiment, the modified CHO cells may be transfected with a high efficiency expression vector (e.g., by transposase-mediated gene integration or other known methodology) containing the wild-type AAT gene. Recombinant pools expressing AAT may be isolated and subsequently, clonal-derived cell lines may be selected by single-cell cloning and expansion. Briefly, modified CHO cells may be co-transfected with the donor GOI or human AAT expression vector and mobilizing or helper transposase vector at a ratio where there is more donor GOI expression vector to transposase vector or less transposase vector to donor GOI expression vector. The donor AAT vector to helper transposase vector ratio may include, but is not limited to, 9:1 (w/w), 9.25:0.75 (w/w), 9.5:0.5 (w/w), 9.75:0.25 (w/w), 9.9:0.1 (w/w), and 10:1 (w/w), or intervening ratios. For example, the ratio of pXLG-6 AAT vector to pXLG-5 transposase vector may include, but is not limited to, 9:1 (w/w), 9.25:0.75 (w/w), 9.5:0.5 (w/w), 9.75:0.25 (w/w), and 9.9:0.1 (w/w). The transfected cells may be maintained under suspension culture, where the medium may be supplemented with a selective agent, such as for example, puromycin at 50 μg/ml and changed daily with puromycin-supplemented medium for about 7 days to about 10 days or until a healthy population of cells has been recovered showing a cell viability of greater than or about 50%, greater than or about 60%, greater than or about 70%, greater than or about 80%, greater than or about 90%, greater than or about 95%, or about 100%. Where the cells have reached a viability of at least about 90%, the cells may be further sub-cultivated in a medium without puromycin since the selection has already occurred. A typical sub-cultivation schedule of about 3 days to about 4 days may continue. The sub-cultivated cells may be tested for successful recombinant protein expression and may be cloned using a limited dilution approach, a single-cell printer (Cytena AG, Freiburg Germany), or both techniques. Up to about 1,000 clonally-derived cell populations may be expanded and investigated for protein production for AAT.

One embodiment may be directed to a method for producing a human recombinant AAT protein including variants thereof, comprising: a) introducing a host cell with an expression vector comprising a nucleic acid fragment containing a nucleic acid sequence which encodes a human AAT protein, including variants thereof, to isolate a transformant, i.e. a recombinant cell, expressing the human recombinant AAT protein including variants thereof; b) culturing the host cells with the transformant, recombinant cells, or expression vector comprising the nucleic acid fragment that comprises a nucleic acid sequence which encodes a AAT under conditions which allow for expression of a human recombinant AAT protein; and c) isolating the human recombinant AAT protein, including variants thereof, from the recombinant cells, thereby producing the human recombinant AAT protein including variants thereof. Another embodiment of the method described herein may provide for the nucleic acid fragment comprising a nucleic acid sequence encoding a human AAT, including variants thereof, that is a CHO-cell codon-optimized sequence.

A further embodiment may provide for the introducing step of the method described here comprising co-transfecting the human AAT expression vector or expression vector comprising AAT variants and an expression vector encoding a transposase, where the transposase expression vector is a helper or mobilizing vector which assists with the incorporation of the gene of interest into at least one host cell genome. Co-transfecting may result in the delivery of the plasmid-excised AAT expression cassette into the genome of the non-recombinant host cells. Another embodiment may be directed to a transposase that is, for example, a piggyBac transposase and a mobilizing vector, helper vector, or an expression vector encoding, for example, a piggyBac transposase, where the transposase gene is introduced into the host cell or host cell culture and not introduced into the genome of the host cells. The expression vector encoding a transposase is a “helper vector” for the incorporation of the gene of interest, i.e., in one embodiment, the AAT expression cassette, into the host cell genome. Another embodiment may be the use of an in-vitro synthetized mRNA preparation that encodes for a transposase, instead of using an expression vector for transposase.

Another embodiment may be directed to a host cell or host cell population that is a eukaryotic cell or a eukaryotic cell population. A further embodiment may provide for a non-recombinant host cell population that is a Chinese hamster ovary (CHO) cell line. Yet another embodiment may be directed to a CHO cell line, where the CHO cell is a recombinant CHO cell line. The CHO cell or CHO cell line may be a CHO cell line that has been modified to produce rapidly-growing hearty or robust cells in a high density which produces high yields of expressed protein. These modified CHO cells may easily scale up from small scale production to large scale manufacturing as well.

In a further embodiment, the non-recombinant and recombinant CHO cell line may be modified to rapidly grow in a culture medium essentially without some or any animal components (i.e., including human components or non-human animal components) or essentially without some or any immune response-inducing human components or non-human animal components. Another embodiment provides for methods disclosed here that are directed to a culturing step of the recombinant CHO cells which occurs essentially without some or any animal (i.e., human or non-human) components. The culturing step occurs in a culture medium, where the culture medium contains less than about 5% (vol/vol), less than about 4% (vol/vol), less than about 3% (vol/vol), less than about 2% (vol/vol), less than about 1% (vol/vol) of any contaminants, or no or essentially no contaminants, where contaminants may include animal-derived components, such as for example, human- and/or non-human-derived components, or any animal-derived components that may trigger an immune response. The culture medium may contain any additives which assist in the growth and expansion of the transformed host cells, including but not limited to feeds, amino acids, and insulin (e.g., human recombinant animal origin free insulin) in an amount that facilitates to expression of recombinant protein isolated from the host cells, such as for example, human AAT protein.

In yet another embodiment, the method described here may be directed to a culturing step that produces or yields about 1 g/L or greater, about 2 g/L or greater, about 3 g/L or greater, about 4 g/L or greater, about 5 g/L or greater, about 6 g/L or greater, about 7 g/L or greater, about 8 g/L or greater, or about 10 g/L or greater, or about 15 g/L or greater of human recombinant AAT protein, about 1 g/L to about 10 g/L of human recombinant AAT protein, about 2 g/L to about 6 g/L of human recombinant AAT protein, or about 3 g/L to about 15 g/L of the human recombinant AAT protein including variants thereof. Another embodiment may be directed to the culturing step that produces about 4 g/L to about 10 g/L of the human recombinant AAT protein including variants thereof.

A further embodiment provides for the method of producing a recombinant AAT protein including variants thereof, wherein the culturing step comprises: selecting the host cell with the nucleic acid fragment expressing the human AAT protein, wherein the selected cells are clonally-derived cells expressing human recombinant AAT protein. The selecting step comprises: a) growing or culturing the clonally-derived recombinant cells expressing human recombinant AAT protein in a culture medium; b) feeding the clonally-derived cells expressing human recombinant AAT protein with at least one feed; c) maintaining the culture medium at a cell culture temperature sufficient to maintain or promote normal, healthy cells; d) modifying or decreasing the cell culture temperature; e) growing or culturing the clonally-derived cells at the decreased cell culture temperature until the cells express the recombinant AAT protein, for example, human recombinant AAT protein at a titer of about 1 g/L or greater, about 2 g/L or greater, about 3 g/L or greater, about 4 g/L or greater, about 5 g/L or greater, about 6 g/L or greater, about 8 g/L or greater, or about 10 g/L or greater, or about 15 g/L or greater, or to a point such that the clonally-derived cells are grown sufficiently to express human recombinant AAT protein including variants thereof at a desired titer of about 1 g/L or greater. Another embodiment may be directed to cells that express human recombinant AAT protein at a titer of about or greater than about 2 g/L, about or greater than about 3 g/L, about or greater than about 4 g/L, or about or greater than about 6 g/L, and in another embodiment, achieving these titers by or at Day 3, Day 5, Day 7, Day 10, Day 14, or Day 17 of cell culturing. A further embodiment may be directed to the host cells that express human recombinant AAT protein including variants thereof at a titer of about or greater than about 6 g/L at Day 17 of cell culturing. In yet a further embodiment, the cell culture temperature during the production phase, i.e. the culture of cells in bioreactors that terminates with the harvests of cells and culture medium, ranges from about 35° C. to about 38° C., or for example, about 37° C., or at the first days of culturing or Day 0 to Day 3 or Day 0 to Day 5, or starting at Day 0 of cell culturing or used interchangeably throughout the description, where Day 0 is the first day of culturing for production, or another appropriate day or range of days that is sufficient for achieving the desired protein levels encoded by the gene of interest, including for example, human recombinant AAT protein. One embodiment may be directed to a shifted, modified, or decreased cell culture temperature ranging from about 25° C. to about 34° C. or for example, about 31° C. to about 33° C., about 31° C., or about 33° C. by, at, or starting at Day 3 or Day 5 of cell culturing, or another appropriate day or range of days that is sufficient for achieving the desired protein levels encoded by the gene of interest, including for example, human recombinant AAT protein. A further embodiment may be directed to a decreased cell culture temperature ranging from about 31° C. to about 33° C. by, at, or starting at Day 3 or by, at, or starting at Day 5 of cell culturing, or another appropriate day or range of days that is sufficient for achieving the desired protein levels encoded by the gene of interest, including for example, human recombinant AAT protein.

In another embodiment, the feeding step of the methods described here provides for at least one feed selected from a neutral feed, an alkaline feed, or another feed that is sufficient to maintain or promote normal healthy cells for achieving the desired protein levels of the gene of interest, including for example, human recombinant AAT protein. Yet a further embodiment provides a neutral feed having a concentration ranging from about 1% to about 10%, about 1% to about 8%, about 1% to about 6%, about 1% to about 5% of the total cell culture volume. Another embodiment may provide for the at least one feed comprising an alkaline feed. In a further embodiment, the alkaline feed may have a concentration ranging from about 0.1% to about 1%, 0.1% to about 0.8%, about 0.1% to about 0.6%, about 0.1% to about 0.5% of the total cell culture volume. One embodiment provides for at least one feed comprising a neutral feed and an alkaline feed. A further embodiment provides for a feed comprising a neutral feed and an alkaline feed in an amount about one-fifteenth ( 1/15), one-tenth ( 1/10), about one-eighth (⅛), about one-sixth (⅙), about one-fifth (⅕) of that of the neutral feed in a total cell culture volume. In one embodiment, the feeding step occurs every day or every other day, or any other feeding schedule which maintains or promotes normal, healthy cells for achieving the desired protein levels of the gene of interest, including for example, human recombinant AAT protein. In another embodiment, the feeding occurs continuously using controlled flow-rates for the neutral feed and/or for the alkaline feed.

Another embodiment may be directed to the methods of producing a recombinant AAT protein where the culturing step during the production phase comprises an osmolarity of the cell culture of about 200 mOsm/kg to about 600 mOsm/kg, about 250 mOsm/kg to about 400 mOsm/kg, about 260 mOsm/kg to about 320 mOsm/kg, about 450 mOsm/kg to about 600 mOsm/kg, about 500 mOsm/kg or greater, about 550 mOsm/kg or greater, or any appropriate osmolarity which maintains or promotes normal, healthy cells for achieving the desired protein levels of the gene of interest, including for example, human recombinant AAT protein. In one embodiment, the osmolarity of the cell culture may be about 550 mOsm/kg or greater by or at Day 5 or later, or any another appropriate day or range of days that is sufficient for achieving the desired protein levels encoded by the gene of interest, including for example, human recombinant AAT protein.

A further embodiment provides a method for producing a human recombinant AAT protein including variants thereof, comprising: a) introducing into a host cell or a host cell population, e.g., eukaryotic, a first nucleic acid sequence encoding a human AAT protein and at least an additional nucleic acid sequence encoding a transposase; b) culturing the host cell or host cell population under conditions which allow expression of the first nucleic acid sequence encoding a human AAT protein, including variants thereof, where the additional nucleic acid sequence encoding, for example, a transposase, such as but not limited to for example, piggyBac, may also be in the cell culture and expressed in order to assist in the incorporation of the gene of interest encoding, i.e., for example, a human AAT, where the host cell is transformed with a nucleic acid sequence encoding a human AAT; c) selecting the host cell with the nucleic acid fragment expressing a human AAT protein, wherein the selected cells are clonally-derived cells expressing human recombinant AAT protein; and d) isolating the recombinant AAT protein, including variants thereof, from the host cell or eukaryotic host cell, thereby producing the human recombinant AAT protein, where the step of isolating may comprise purifying the human recombinant AAT protein.

Another embodiment may provide for a eukaryotic host cell or a eukaryotic cell population transformed with a nucleic acid sequence encoding a human AAT protein. In another embodiment, the step of introducing comprises co-transfecting the host cell with a vector containing the first nucleic acid sequence encoding an AAT protein, including variants thereof, and with a vector containing the additional nucleic acid sequence encoding a transposase, such as but not limited to, a piggyBac, Tol-2, Sleeping Beauty, Leap-In, and any other “cut-and-paste” transposase, or the like. Yet a further embodiment of the isolating step may provide a step of purifying the desired recombinant AAT protein. The purifying step may be performed by at least one of, but is not limited to, any one or more of the techniques of: affinity chromatography including, for example, antibody- or ligand-based affinity chromatography, size exclusion chromatography, ion exchange chromatography, hydrophobic interaction chromatography, reverse phase chromatography, gel filtration, magnetic bead separation, selective precipitation, molecular weight-based membrane filtration or exclusion, buffer exchange, virus filtration, pH-based inactivation of viruses, and the like.

A further embodiment of the invention may be directed to the method of producing a human recombinant AAT protein, including variants thereof, where the method comprises a culturing step in a culture medium that is essentially free of animal-derived components (i.e., human or non-human animal components) or proteins that induce an immune response, such as for example, human immunoglobulins, human serum albumin, non-human animal proteins, non-human animal immunoglobulins, non-human serum albumin, or any other known plasma protein that may be considered a contaminant. In a further embodiment, the method of producing a human recombinant AAT protein, including variants thereof, where the method comprises a culturing step in a culture medium that contains less than about 5% (vol/vol) of animal-derived components, less than about 4% (vol/vol) of animal-derived components, less than about 3% (vol/vol) of animal-derived components, less than about 2% (vol/vol) of animal-derived components, less than about 1% (vol/vol) of animal-derived components. Yet another embodiment is directed to the method of producing a human recombinant AAT protein, including variants thereof, where the method comprises a culturing step in a culture medium, where the culture medium may or may not comprise a human recombinant animal origin free insulin. A further embodiment may provide for a method of producing a human recombinant AAT protein having a purity of about 95% or greater or about 98% or greater, where the purity of the human recombinant AAT protein may be substantially or essentially free from components, which naturally accompany the human recombinant AAT protein, are used to produce the human recombinant AAT protein or are degradation products from producing the human recombinant AAT protein. Contaminant components may be those materials that differ from the desired human recombinant AAT protein, or those naturally occurring or present materials which would interfere with research, diagnostic or therapeutic uses for the protein, and may include enzymes, hormones, and other proteinaceous or non-proteinaceous solutes. The purity of the human recombinant AAT protein may be determined by any art-recognized method of analysis (e.g., polyacrylamide gel electrophoresis, HPLC, silver stained gel, and the like). Generally, the purity of the human recombinant AAT protein means that the protein has been increased in purity, such that it exists in a form that is purer than when in its natural environment and/or when initially produced and/or synthesized. Typically, the purity of an isolated human recombinant AAT protein may be about 60%, about 70%, about 80%, about 90%, about 95%, or greater than about 90%, about 92%, about 94%, about 95%, about 96%, about 98%, or about 100%.

### Cell Line and Process Optimization

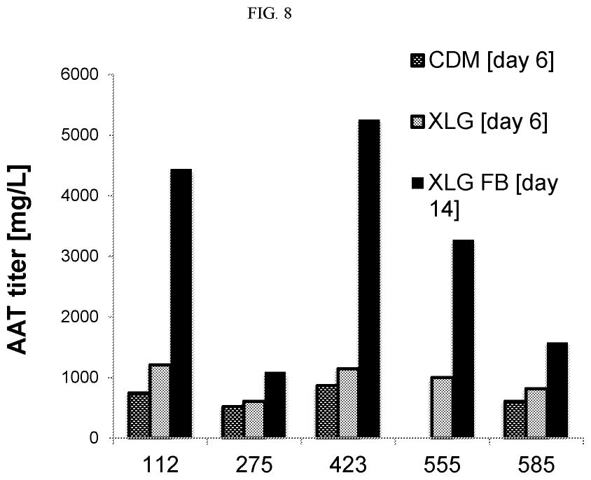

In one embodiment, clonally-derived cell lines comprising the desired recombinant AAT, including variants thereof, may be cultivated under high-throughput conditions. For example, the clonally-derived cell lines described here, including for example, cell lines 30, 112, and 423, were cultivated under numerous conditions using a high-throughput culture system while applying small scale (10 ml) cultures in orbitally shaken 50 ml-OrbShake tubes (e.g., TPP® TubeSpin® bioreactor tubes, TubeSpin® Bioreactor 50, Trasadingen, Switzerland; or similar product). These tubes are provided with a ventilated cap and are typically shaken at 180 rpm with a displacement radius of 50 mm within a CO₂-controlled, humidified incubator shaker (Kuhner Shaker, Birsfelden, Switzerland). In one embodiment, the clonally-derived cell lines, comprising clonally-derived cells, may be cultivated under high-throughput conditions where cell viability and growth under use of numerous media compositions, feed compositions, timing of the additions, volumes of feed additions, temperature shifts, and other process conditions were studied. In another embodiment, the clonally-derived cells may be cultivated under high-throughput conditions and processed on a larger scale, where it is understood that the conditions for scaling up the process from small scale to large scale is direct or essentially direct. An example of various procedures useful in producing the desired recombinant AAT including variants thereof may be found in FIG. 8, which compares the use of different feed strategies, i.e. feed volumes, feed types, feed timings, etc. and temperature shifts in a fed-batch process. The feeds (7A and 7B; HyClone™ Cell Boost 7a, HyClone™ Cell Boost 7b, Catalog Numbers SH31026.01 (RRG168030, SH31027.01) shown in the example are commercially available and are given in volumes in percent of the total effective cell culture volume and are provided every day (ED) or every other day (EOD). Each of the indicated culture condition has been executed in triplicate and the error-bars are indicated as thin lines over the columns. An embodiment of the invention may be directed to titers of recombinant AAT including variants thereof produced by clonally-derived cells under process conditions such as those identified in FIG. 8, conditions 1 and 3, such that AAT titers reach more than about 6 g/L by Day 17. For example, the clonally-derived cell line 112 was elevated to more than 6 g/L on Day 17 under process conditions 1 and 3.

Yet another embodiment may be directed towards the large-scale cell culturing and optimization using a bioreactor. In one embodiment, the use of chemically-defined feeds, such as a feed comprising inorganic salts, amino acids, and vitamins (e.g., XLG Feed A (Feed A4CHO); ExcellGene S.A.; etc.) and a feed comprising organics and other beneficial components (e.g., XLG Feed B (Feed B4CHO); ExcellGene S.A., etc.) may be added to the bioreactor at different time points during the production phase. These feeds may be added in the same or different volumes, i.e., fractions of the working volume of a production bioreactor. A person of ordinary skill in the art would understand how to modify process conditions and amounts in order to produce a defined quality of recombinant AAT in large amounts.

A further embodiment provides a method of producing recombinant AAT comprising: culturing or growing clonally-derived cells expressing recombinant AAT; feeding clonally-derived cells expressing recombinant AAT with at least one feed, where the feeding occurs continuously or discontinuously, where feeding may occur using controlled flow-rates; maintaining a cell culture temperature; shifting the maintained cell culture temperature to a shifted cell culture temperature; growing the cells at the decreased cell culture temperature until the cells express recombinant AAT at a titer of greater than about 1 g/L, greater than about 2 g/L, greater than about 3 g/L, greater than about 4 g/L, greater than about 5 g/L, greater than about 6 g/L, greater than about 7 g/L, greater than about 8 g/L, greater than about 9 g/L, or greater than about 10 g/L; or at least about 1.5 g/L, at least about 2.5 g/L, at least about 3.5 g/L, at least about 4.5 g/L, at least about 5.5 g/L, at least about 6.5 g/L, at least about 7.5 g/L, at least about 8.5 g/L, at least about 9.5 g/L, or at least about 10.5 g/L; or ranging from about 1 g/L to about 10 g/L, ranging from about 2 g/L to about 10 g/L, ranging from about 3 g/L to about 10 g/L, ranging from about 4 g/L to about 10 g/L, ranging from about 5 g/L to about 10 g/L, ranging from about 6 g/L to about 10 g/L, ranging from about 7 g/L to about 10 g/L, ranging from about 8 g/L to about 10 g/L, or ranging from about 9 g/L to about 10 g/L. In a further embodiment, the clonally-derived cells are fed every day. Yet another embodiment may be directed to feeding the clonally-derived cells every other day. A further embodiment may be directed to feeding the clonally-derived cells every 3 days, or any other feeding schedule that benefits the overall health of the cells and thereby increases the final harvested product titer of recombinant AAT.

One embodiment may be directed to growing clonally-derived cells expressing recombinant AAT for a number of days sufficient to reach a recombinant AAT titer of greater than about 1 g/L, greater than about 2 g/L, greater than about 3 g/L, greater than about 4 g/L, greater than about 5 g/L, greater than about 6 g/L, greater than about 7 g/L, greater than about 8 g/L, greater than about 9 g/L, or greater than about 10 g/L; or at least about 1.5 g/L, at least about 2.5 g/L, at least about 3.5 g/L, at least about 4.5 g/L, at least about 5.5 g/L, at least about 6.5 g/L, at least about 7.5 g/L, at least about 8.5 g/L, at least about 9.5 g/L, or at least about 10.5 g/L; or ranging from about 1 g/L to about 10 g/L, ranging from about 2 g/L to about 10 g/L, ranging from about 3 g/L to about 10 g/L, ranging from about 4 g/L to about 10 g/L, ranging from about 5 g/L to about 10 g/L, ranging from about 6 g/L to about 10 g/L, ranging from about 7 g/L to about 10 g/L, ranging from about 8 g/L to about 10 g/L, or ranging from about 9 g/L to about 10 g/L. The number of days sufficient to obtain a recombinant AAT titer of greater than about 1 g/L may range from 7 days to 21 days, or the number of days may be at least 7 days, at least 11 days, at least 14 days, at least 17 days, or at least 21 days.

In a further embodiment, the cell culture temperature during the production phase may be maintained at a temperature ranging from about 35° C. to about 38° C., including but not limited to, at about 35° C., at about 36° C., at about 37° C., at about 38° C., or less than about 39° C. Another embodiment may be directed to a shifted cell culture temperature ranging from about 24° C. to about 34° C., or any temperatures at or in between, including but not limited to, at about 24° C., at about 25° C., at about 26° C., at about 27° C., at about 28° C., at about 29° C., at about 30° C., at about 31° C., at about 32° C., at about 33° C., or at about 34° C. Another embodiment may be directed to a cell culture temperature that is maintained at about 37° C. for the first 2 days or the first 3 days, or portions of day 3 thereof. A further embodiment may be directed to a decreased cell culture temperature of about 33° C., about 32° C., about 31° C., about 30° C., about 29° C., about 28° C., about 27° C., about 26° C., about 25° C., or about 24° C. to about 25° C., where the decreased cell culture temperature occurs on day 3, or a portion of day 3 thereof. Yet, a further embodiment may be directed to a decreased cell culture temperature of about 31° C. occurring on day 5, or a portion of day 5 thereof. In another embodiment, the decreased cell culture temperature comprises more than one decreased cell culture temperature, where a first decreased cell culture temperature of about 33° C., about 32° C., about 31° C., about 30° C., about 29° C., about 28° C., about 27° C., about 26° C., about 25° C., or about 24° C. to about 25° C. occurs on day 3, or a portion of day 3 thereof, and a second decreased cell culture temperature occurs on day 5, or portions of day 5 thereof by about 2° C. to about 3° C. below the previously used temperature after the first temperature shift. Alternative days to those mentioned here are also contemplated for the temperature shift to occur, as long as the clonally-derived cells expressing recombinant AAT are healthy, i.e., not dying or dead.

In another embodiment, the cell culture during the production phase comprising host cells incorporated with rAAT described here, may be maintained to have an osmolarity ranging of about 200 mOsm/kg to about 600 mOsm/kg, about 250 mOsm/kg to about 400 mOsm/kg, about 260 mOsm/kg to about 320 mOsm/kg, about 450 mOsm/kg to about 600 mOsm/kg, about 500 mOsm/kg or greater, about 550 mOsm/kg or greater, or any appropriate osmolarity which maintains or promotes normal, healthy cells for achieving the desired protein levels of the gene of interest, including for example, human recombinant AAT protein. Changes in osmolarity over the days in culture are also contemplated as osmolarity increases during the course of cell culture and nutrients in fed-batch cultures also increase the osmolarity. Another embodiment may be directed to increasing osmolarity of the cell culture to about 550 mOsm/kg or greater by or at Day 5 or later, or any appropriate day that allows for normal, healthy cells for achieving the desired protein levels of the gene of interest, including for example, human recombinant AAT protein as described here.

Yet a further embodiment may be directed to a feed that is animal component-free, or chemically defined, optimized for high-yield protein production, including but not limited to, in fed-batch processes, free of growth factors, animal tissue-derived peptides, animal tissue-derived hydrolysates, phenol red, or 2-mercaptoethanol. In yet another embodiment, the feed may comprise of a neutral or close to neutral pH, i.e., a neutral feed, where the neutral feed may, in some embodiments, contain amino acids, vitamins, salts, and glucose. In a further embodiment, the feed may be chemically defined, or may contain non-animal derived components, such as hydrolysates from plant seeds, from certain cereals (wheat), from certain beans or peas (soybean) or the like. In a further embodiment, the feed may comprise of an alkaline pH, i.e., an alkaline feed, where the alkaline feed may, in some embodiments, contain a concentrated solution of amino acids. Another embodiment may be directed to feeding the clonally-derived cells expressing recombinant AAT with a combination of feeds, where the combination of feeds may include the neutral feed and the alkaline feed, fed either simultaneously, essentially simultaneously, sequentially, or essentially sequentially. Moreover, feeding the cell culture with additives, including but not limited to, feeds, nutrients, amino acids, and the like, may occur continuously or discontinuously, where feeding may occur using controlled flow-rates, and a schedule comprising feeding at least one feed every day or every other day, or any other feeding schedule that maintains or promotes normal, healthy cells for achieving the desired protein levels of the gene of interest, including for example, human recombinant AAT protein.

A further embodiment may be directed to a combination of feeds, where the neutral feed has a concentration ranging from about 1% to about 8% of the total cell culture volume, or any percentages at or in between, including but not limited to, at about 1.8%, at about 3.6%, or at about 7.1%; and where the alkaline feed has a concentration ranging from about 0.1% to about 0.8% of the total cell culture volume, or any percentages at or in between, including but not limited to, at about 0.18%, at about 0.36%, or at about 0.71%. Another embodiment may be directed to concentrations of feed, where the alkaline feed is in an amount of about one-tenth ( 1/10) the amount of the neutral feed. For example, the percentage of the neutral feed ranges from about 1.8% to about 7.1% and the percentage of the alkaline feed ranges from about 0.18% to about 0.71%, i.e., one-tenth of the percentage of the neutral feed, where the feed percentages are in relation to the total cell culture volume that may include at least one of: the cells, culture medium, feeds, and any other nutrients or additives for culturing the cells to maintain or produce normal, healthy cells for achieving the desired protein levels of the gene of interest, including for example, human recombinant AAT protein.

In one embodiment, a method of producing recombinant AAT may be directed to: growing clonally-derived cells expressing recombinant AAT; feeding the clonally-derived cells expressing recombinant AAT a feed comprising: a neutral feed, such as for example, 7A feed (HyClone™ Cell Boost 7a) and an alkaline feed, such as for example, 7B feed (HyClone™ Cell Boost 7b) every other day; maintaining a culture temperature of 37° C. from day 0 to day 3, including a portion of day 3, i.e., from greater than 0 hours (day 0) to 72 hours, 78 hours or 84 hours or any timing in between the provided hours (day 3) shifting the culture temperature to 33° C. on day 3, when the previously provided timing of the first temperature setting ends, purifying recombinant AAT from the cells; and collecting the purified recombinant AAT. In one embodiment, the concentration of the neutral feed is about 7.1% of the total volume of cell culture and the concentration of the alkaline feed is about 0.71% of the total volume of cell culture. An embodiment of the invention may be directed to feeding a feed comprising a combination of a neutral feed and an alkaline feed, where the feed is fed every other day to the clonally-derived cells expressing recombinant AAT, where the cell culture temperature is maintained at about 37° C. from day 0 to day 3, or a portion of day 3 thereof, and where the decreased cell culture temperature is about 33° C. starting on day 3, or a portion of day 3 thereof. In another embodiment further to the feeding schedule described here is a media used to grow the clonally-derived cells expressing recombinant AAT, where the media provide additional nutrients, amino acids, metals, and the like, which enhance the cell culture conditions, and may include, for example, a chemically defined medium such as XLG_E21_07 (ExcellGene SA). Further embodiments of the invention may include the XLG_E21_07 medium by modifying the concentration of several components, such as increasing the concentration of glucose, zinc, asparagine, glutamic acid, and phosphate, simply by, for example, adding stock solutions of higher concentrations in small volumes for adjustment. The concentrations of these exemplary five components can be modified according to the following ranges found in TABLE 2:

TABLE 2

| | CONCENTRATION | CONCENTRATION |
|---|---|---|
| COMPONENT | FROM | TO |
| Glucose | about 2 g/L | about 30 g/L |
| Zinc | about 0.1 mg/L | about 10 mg/L |
| Asparagine | about 500 mg/L | about 7000 mg/L |
| Glutamic Acid | about 100 mg/L | about 3000 mg/L |
| Phosphate | about 100 mg/L | about 3000 mg/L |

A further embodiment may be directed to a method of producing recombinant AAT, comprising: culturing or growing clonally-derived cells expressing recombinant AAT; feeding the clonally-derived cells expressing recombinant AAT at least one feed including but not limited to: a neutral feed, such as for example, HyClone™ Cell Boost 7A feed and an alkaline feed, such as for example, HyClone™ Cell Boost 7B feed every day, where the alkaline feed is present in an amount of about 1/10 that of the neutral feed, and the amount (vol/vol) of the feeds are based on the total cell culture volume; maintaining a culture temperature of 37° C. from day 0 to day 3, whereby the inoculation of the production vessel with fresh cells from the “N-1” bioreactor (pre-culture or seed bioreactor) is defined as the start of day 0, and then shifting the culture temperature to 33° C. on day 3 (i.e, 72 hours after the start of the production culture); purifying recombinant AAT expressed by the cells; and collecting the purified recombinant AAT. In one embodiment, the concentration of the neutral feed is about 3.6% of the total volume of cell culture and the concentration of the alkaline feed is about 0.36% of the total volume of cell culture. An embodiment of the invention may be directed to feeding a feed comprising a combination of a neutral feed and an alkaline feed, where the feed is fed every day to the clonally-derived cells expressing recombinant AAT, where the cell culture temperature is maintained at about 37° C. from day 0 to day 3, or a portion of day 3 thereof, and where the decreased cell culture temperature is about 33° C. starting on day 3, or a portion of day 3 thereof. The feed, either alone or in combination, that may be used in the methods and processes described here include, but are not limited to, those presented in the table of feeds, TABLE 3:

TABLE 3

| | | CATALOG |
|---|---|---|
| FEED | MANUFACTURER | No |
| HyClone ™ Cell Boost 7a | HyClone ™ | SH31026.01 |
| | | (RRG168030) |
| HyClone ™ Cell Boost 7b | HyClone ™ | SH31027.01 |
| Cellvento ™ Feed-210 | Merck Millipore | 1.02488.0005 |
| Cellvento ™ Feed-220 | Merck Millipore | 1.02578.0003 |
| Cellvento ™ Feed-200 | Merck Millipore | 1.01883.0003 |
| Cellvento ™ 4Feed | Merck Millipore | 1.03796.0005 |
| L-Cyteine + L-Tyrosine (Feed B) | Merck Millipore | 1.02735.0100 |
| BalanCD CHO FEED 1 | Irvine | 91127 |
| BalanCD CHO FEED 2 | Irvine | 91129 |
| BalanCD CHO FEED 3 | Irvine | 99471 |
| BalanCD CHO FEED 4 | Irvine | 94134 |
| Efficient Feed ™ A + AGT (3X) | Thermo Fisher | A25023-04 |
| Efficient Feed ™ B + AGT (3X) | Thermo Fisher | A25030-04 |
| Efficient Feed ™ C + AGT (2X) | Thermo Fisher | A25031-04 |
| PowerFeed A | Sartorius | BE 02-044Q |
| CHO Xtreme Feed ™ | Sartorius | BE02-056Q |
| PowerFeed Advance | Sartorius | |
| Ex-Cell Advanced CHO Feed 1 | Sigma | 24367-1L |
| (with glucose) | | |
| Ex-Cell CHOZN Platform Feed | Sigma | 24331C-10L |
| EX-Cell ™ Hydrolysate | Sigma | 24700C-100G |
| Cell Boost TM 1 (R05.2) | GE | SH30584.02 |
| Cell Boost TM 2 (R15.4) | GE | SH30596.01 |
| Cell Boost TM 3 (JM 3.5) | GE | SH30825.01 |
| Cell Boost TM 4 (PS307) | GE | SH30857.01 |
| Cell Boost TM 5 (CN-F) | GE | SH30865.01 |
| Cell Boost TM 6 (CN-T) | GE | SH30866.01 |
| Feed A4CHO (CDM) | ExcellGene SA | |
| Feed B4CHO (CDM) | ExcellGene SA | |
| Feed 3CHO (ACF) | ExcellGene SA | |

In another embodiment further to the feeding schedule described here are media used to grow the clonally-derived cells expressing recombinant AAT, where the media may be free of any components derived from animals, such as but not limited to fetal bovine serum (FBS), for example a chemically defined medium, such as medium XLG_E21_07 (ExcellGene SA). Non-limiting examples of media, either alone or in combination, useful in the methods and processes of producing recombinant AAT described herein include also those presented in the table of media, where chemically derived (CD) and animal component-free (ACF) are specified, i.e. TABLE 4:

TABLE 4

| | CD/ | | |
|---|---|---|---|
| MEDIUM | ACF | MANUFACTURER | CATALOG NO. |
| CD CHO Medium (1x), liquid | CD | GIBCO (Invitrogen) | 10743-011 |
| CD FortiCHO | CD | GIBCO (Invitrogen) | A11483-01 |
| CD OptiCHO ™ Medium | CD | GIBCO (Invitrogen) | 12681-029 |
| HyClone ™ CDM4 PERMAb | CD | HyClone ™ (GE Healthcare) | SH30871.02 |
| HyClone ™ CDM4CHO | CD | HyClone ™ (GE Healthcare) | SH30558.02 |
| HyClone ™ CDM4MAb | CD | HyClone ™ (GE Healthcare) | SH30802.02 |
| EX-CELL ™ CD CHO Fusion | CD | SAFC Biosc (SIGMA) | 14365C |
| ActiPro | CD | HyClone ™ (GE Healthcare) | SH31039.02 |
| CD 293 | CD | GIBCO (Invitrogen) | 11913-01 |
| PowerCHO1 CD | CD | Lonza | 12-770Q |
| PowerCHO2 CD | CD | Lonza | BE12-771Q |
| PowerCHO3 CD | CD | Lonza | 12-772Q |
| ProNSO 1 | CD | Lonza | 12-773 Q |
| ProNSO 2 CD | CD | Lonza | 12-774Q |
| POWER CHO Advance | CD | Lonza | 12-927Q |
| CD DG 44 medium | CD | GIBCO (Invitrogen) | 12610-010 |
| PeproGrow CD-CHO Medium | CD | PEPROTECH | AF-CD-CHO |
| EX-CELL ™ CD CHO3 Medium | CD | SAFC Biosc (SIGMA) | C1490-1L |
| EX-CELL ™ Advanced ™ CHO | CD | SAFC Biosciences (SIGMA) | 14366C-1000 |
| Feed- batch Medium | | | ML |
| IS CHO CD XP | CD | Irvine | 91120 |
| BalanCD ™ CHO GROWTH A | CD | Irvine | 91128 |
| EX-CELL ™ CHOZN Platform | CD | SAFC Biosci (SIGMA) | 14330C-1L |
| Medium | | | |
| Hycell CHO Medium | CD | HyClone ™ (GE Healthcare) | SH30934.01 |
| Dynamis ™ Medium | CD | GiBCO (Invitrogen) | A26615-01 |
| Cellvento ™ CHO-100 | CD | Merck Millipore | 1.00899.0010 |
| Cellvento ™ CHO-200 | CD | Merck Millipore | 1.01885.0010 |
| Cellvento ™ CHO-210 | CD | Merck Millipore | 1.02485.0010 |
| Cellvento ™ CHO-220 | CD | Merck Millipore | 1.02680.0500 |
| Pro293s-CDM | CD | Lonza | BE02-025Q |
| CD Hybridoma | CD | GIBCO (Invitrogen) | 11279-023 |
| EX-CELL ™ CD Hybridoma | CD | SAFC Biosciences (SIGMA) | H4409-1L |
| Medium | | | |
| CHOMACS CD | CD | Miltenyi Biotec GmbH | 170-077-001 |
| SHEFF-CHO CD COMPLETE | CD | Kerry | S-870392 |
| ASF CHO 3.2 | CD | AJINOMOTO | |
| EX-CELL ™ 620-HSF | ACF | SAFC Biosc (SIGMA) | 14621C |
| Hybridoma | | | |
| EX-CELL ™ ACF CHO medium | ACF | SAFC Bios (SIGMA) | C5467 |
| EX-CELL ™ CHO DHFR- | ACF | SAFC Biosc(SIGMA) | C8862 |
| medium Animal Component Free | | | |
| EX-CELL ™ 325 PF CHO | ACF | SAFC Biosc(SIGMA) | 14340C |
| EX-CELL ™ 302 SF for CHO | ACF | SAFC Biosc(SIGMA) | 14324C |
| Adenovirus expression medium | ACF | GIBCO (Invitrogen) | 12582-011 |
| (AEM) | | | |
| FreeStyle CHO expression | ACF | GIBCO (Invitrogen) | 12651-014 |
| Hybridoma-SFM serum-free | ACF | GIBCO (Invitrogen) | 12045-084 |
| Protein Expression Medium | ACF | GIBCO (Invitrogen) | 12661-013 |
| (PEM) | | | |
| HyQ ADCF MAB | ACF | HyClone ™ (Socochim) | SH 30349.02 |
| ProCHO5 | ACF | Lonza | 4SP144 |
| PowerCHO -GS | ACF | Lonza | BE12-776Q |
| EX-CELL ™ 293 | ACF | SAFC Biosc (SIGMA) | 14571C |
| EX-CELL ™ CHO5 Medium | ACF | SAFC Biosc(SIGMA) | C0363-1L |
| EX-CELL ™ GTM-3 ACF | ACF | SAFC Biosc(SIGMA) | G9916 |
| FreeStyle ™ 293 Expression | ACF | GIBCO (Invitrogen) | 12338-018 |

Another embodiment may be directed to the use of various media and/or feed compositions and any suitable combinations thereof, as well as combinations of the media and feed combinations with any other process condition including but not limited to certain defined pH values, oxygen level and/or oxygen-providing sparging (gas-providing) regimen (oxygen and/or air, with or without providing nitrogen and/or CO2 simultaneously), temperature, and osmolarity that may result in an increase in the volumetric productivity of recombinant AAT in cell culture to a titer of greater than or equal to about 6 g/L, greater than or equal to about 8 g/L, or greater than or equal to about 10 g/L.

Briefly, the pH value of the culture conditions during the production phase may range from about pH 6.25 to about pH 7.5; about pH 6.5 to about pH 7.3; about pH 6.7 to about pH 7.3; about pH 6.7 to about pH 7.1; about pH 6.8 to about pH 7; greater than or equal to about pH 6.5; greater than or equal to about pH 6.7; greater than or equal to about pH 6.8; less than or equal to about pH 7.5; less than or equal to about pH 7.3; less than or equal to about pH 7.1; or less than or equal to about pH 7. The oxygen level and/or sparging regimen of the culture conditions may range from about 20% oxygen relative to air saturation to about 60% oxygen relative to air saturation; about 25% oxygen relative to air saturation to about 55% oxygen relative to air saturation; about 20% oxygen relative to air saturation to about 60% oxygen relative to air saturation; greater than or equal to about 20% oxygen relative to air saturation; greater than or equal to about 25% oxygen relative to air saturation; greater than or equal to about 30% oxygen relative to air saturation; greater than or equal to about 35% oxygen relative to air saturation; greater than or equal to about 40% oxygen relative to air saturation; greater than or equal to about 45% oxygen relative to air saturation; greater than or equal to about 50% oxygen relative to air saturation; greater than or equal to about 55% oxygen relative to air saturation; less than or equal to about 60% oxygen relative to air saturation; less than or equal to about 55% oxygen relative to air saturation; less than or equal to about 50% oxygen relative to air saturation; less than or equal to about 45% oxygen relative to air saturation; less than or equal to about 40% oxygen relative to air saturation; less than or equal to about 35% oxygen relative to air saturation; less than or equal to about 30% oxygen relative to air saturation; or less than or equal to about 25% oxygen relative to air saturation. The temperature of the culture conditions may also range from about 26° C. to about 38° C.; about 28° C. to about 38° C.; about 29° C. to about 37° C.; about 31° C. to about 37° C.; about 34° C. to about 37° C.; about 31° C. to about 33° C.; greater than or equal to about 26° C.; greater than or equal to about 28° C.; greater than or equal to about 31° C.; greater than or equal to about 33° C.; greater than or equal to about 34° C.; greater than or equal to about 37° C.; less than or equal to about 38° C.; less than or equal to about 37° C.; less than or equal to about 36° C.; less than or equal to about 34° C.; less than or equal to about 33° C.; or less than or equal to about 31° C.

In one embodiment, the cell culture harvest titers of a recombinant AAT may have a titer of: at least about 1 g/L, at least about 3 g/L, at least about 4 g/L, at least about 6 g/L, at least about 8 g/L, and at least about 10 g/L; about 3 g/L, about 4 g/L, about 5 g/L, about 6 g/L, about 7 g/L, about 8 g/L, about 9 g/L, and about 10 g/L; greater than or equal to 1 g/L, greater than or equal to about 2 g/L, greater than or equal to about 2.5 g/L, greater than or equal to about 3.5 g/L, greater than or equal to about 4.5 g/L, greater than or equal to about 5.5 g/L, greater than or equal to about 6.5 g/L, greater than or equal to about 7.5 g/L, greater than or equal to about 8.5 g/L, greater than or equal to about 9.5 g/L, and greater than or equal to about 10.5 g/L.

Yet a further embodiment may be directed to a method of producing recombinant AAT, where the recombinant AAT may have a high number of terminal sialic acids, such that the circulation half-life of recombinant AAT may be increased in a subject who received a dose of recombinant AAT.

In one embodiment, a method for producing a human recombinant AAT protein comprises culturing a host cell with a first nucleic acid sequence encoding a human AAT protein and at least a second nucleic acid sequence encoding a transposase, wherein the culturing step occurs at a first period of time at a first temperature and at a second period of time at a second temperature, and optionally at a third period of time at a third temperature. Another embodiment of the method may provide for the second temperature that is less than the first temperature (e.g., lower by at least: about 1° C., about 2° C., about 3° C., about 4° C., about 5° C., about 6° C., about 8° C., about 10° C., etc.). A further embodiment of the method may provide for the third temperature that is less than either the second temperature or the first temperature (e.g., lower by at least: about 1° C., about 2° C., about 3° C., about 4° C., about 5° C., about 6° C., about 8° C., about 10° C., etc.). Another embodiment of the method provides for the first temperature, the second temperature, and/or the third temperature that is greater than room temperature (e.g., about 15° C., about 20° C., about 21° C., about 22° C., about 23° C., about 24° C., about 25° C., etc.). In yet another embodiment, the first period of time of culturing may be for at least about 1 day, about 2 days, about 3 days, about 4 days, about 5 days, about 6 days, about 10 days, about 15 days, about 1-2 days, about 1-3 days, about 1-4 days, about 1-5 days, about 1-7 days, about 1-10 days, about 1-15 days, about 1-16 days, about 1-17 days, about 1-18 days, about 1-20 days, etc. A further embodiment provides for the second period of time of culturing, where the second period of time may be for at least about 1 day, about 2 days, about 3 days, about 4 days, about 5 days, about 6 days, about 10 days, about 15 days, about 1-2 days, about 1-3 days, about 1-4 days, about 1-5 days, about 1-7 days, about 1-10 days, about 1-15 days, about 1-16 days, about 1-17 days, about 1-18 days, about 1-20 days, etc. Yet another embodiment may be directed to a third period of time of culturing, where the third period of time may be for at least about 1 day, about 2 days, about 3 days, about 4 days, about 5 days, about 6 days, about 10 days, about 15 days, about 1-2 days, about 1-3 days, about 1-4 days, about 1-5 days, about 1-7 days, about 1-10 days, about 1-15 days, about 1-16 days, about 1-17 days, about 1-18 days, about 1-20 days, etc. A further embodiment may be directed to the first period of time comprising about 1-20 days, about 1-18 days, about 1-17 days, about 1-16 days, about 1-15 days, about 1-10 days, about 1-6 days, about 1-5 days, about 1-4 days, about 1-3 days, or about 1-2 days; the second period of time comprising about 1-20 days, about 1-18 days, about 1-17 days, about 1-16 days, about 1-15 days, about 1-10 days, about 1-6 days, about 1-5 days, about 1-4 days, about 1-3 days, or about 1-2 days; and optionally, a third period of time comprising about 1-20 days, about 1-18 days, about 1-17 days, about 1-16 days, about 1-15 days, about 1-10 days, about 1-6 days, about 1-5 days, about 1-4 days, about 1-3 days, or about 1-2 days.

Yet a further embodiment may provide for a method for producing a human recombinant AAT protein comprises culturing a host cell with a first nucleic acid sequence encoding a human AAT protein and using in a first phase for generation of the host cell at least a second nucleic acid sequence encoding a transposase, wherein the culturing step occurs at a first period of time at a first temperature and at a second period of time at a second temperature, wherein a first feed and a second feed are administered every other day or every day, the first period of time comprises about 1-10 days, about 1-6 days, about 1-5 days, about 1-4 days, about 1-3 days, about 1-2 days at a first temperature ranging from about 31° C. to about 37° C., about 33° C. to about 37° C., and the second period of time comprises about 1-20 days, about 1-18 days, about 1-17 days, about 1-15 days, about 1-10 days, about 1-8 days, about 1-7 days, about 1-6 days, about 1-5 days, about 1-4 days, about 1-3 days, about 1-2 days at a second temperature ranging from about 31° C. to about 37° C., about 33° C. to about 37° C., and if the method comprises a third period of time at a third temperature, the third period of time comprises about 1-20 days, about 1-18 days, about 1-17 days, about 1-15 days, about 1-10 days, about 1-8 days, about 1-7 days, about 1-6 days, about 1-5 days, about 1-4 days, about 1-3 days, about 1-2 days at a third temperature ranging from about 31° C. to about 37° C., about 33° C. to about 37° C.

Thus, one solution to the problem of insufficient supply of plasma-derived AAT was directed to embodiments for producing very high yields from recombinant mammalian cells expressing recombinant AAT. In embodiments of the invention, process conditions for growth and productivity while taking into consideration the diverse physiological metabolism of clonally derived cell populations were used and media and feed compositions that support advantageous phenotypes of cells for bioreactor-based manufacturing of proteins were identified.

### AAT Product Characterization and Activity

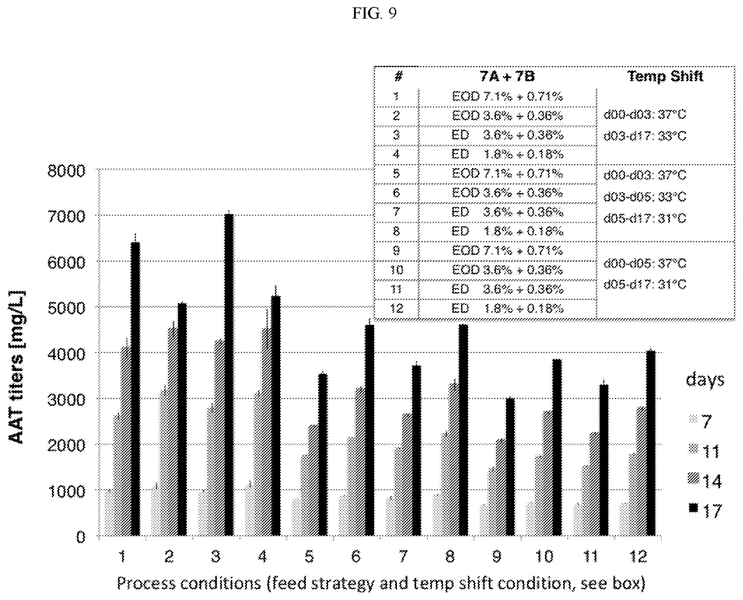

Recombinant AAT was purified from harvested cell culture fluids by a 2-step chromatographic procedure and subsequently analyzed by size exclusion chromatography. The main peak in FIG. 9 shows the purified recombinant AAT material fraction eluting at about 12 minutes to about 14 minutes. Analytical HPLC was executed to obtain the glycosylation pattern of recombinant AAT, as produced by one of the early production runs (FIG. 9). Also, analytical IEF was executed with materials obtained from different process conditions in order to see how such process conditions would affect the overall structural representation of the glycosylations of AAT (data not shown). A comparison between plasma-derived AAT and recombinant AAT as produced by CHO cells concerns oxidized methionines. Wildtype AAT contains 8 methionines that could be a target of oxidation. Particularly methionine 358 and methionine 351 have been reported to be susceptible to oxidation, and when oxidized, the methionines seem to have a profound effect on AAT, reducing its capacity to inhibit elastase and to be responsible for a pro-inflammatory response (Li Z, et al. Am J Physiol Lung Cell Mol Physiol. 297(2):L388-400, 2009). Accordingly, oxidation of some methionine residues in AAT may result in a decreased ability to inhibit neutrophil elastase, thereby contributing to the pathogenesis of diseases or conditions that have a deficiency of AAT. Thus, in one embodiment of the invention, conditions during the production phase in the bioreactor are being used that minimize oxidation of methionines and thus an active, glycosylated recombinant AAT protein or variant recombinant AAT protein may have fewer than 8 methionine oxidation targets, fewer than 6 methionine oxidation targets, fewer than 4 methionine oxidation targets, fewer than 3 methionine oxidation targets, or a variant recombinant AAT protein having fewer than 7 oxidizable methionines, fewer than 5 oxidizable methionines, fewer than 3 oxidizable methionines, or fewer than 1 oxidizable methionine. Another embodiment of the invention may be directed to an active recombinant AAT having fewer oxidized methionines than that of a plasma-derived AAT, or where the level of oxidized methionines in plasma-derived AAT is greater than that in recombinant AAT produced in the modified CHO cells described here. Such an embodiment of the invention may be the provisioning of oxygen to the cells in culture under avoidance of use of pure oxygen gas, but to use only purified air during the cell culture production phase.

Another embodiment may be directed to a human recombinant AAT protein including variants thereof, that may have benefits over the human wild-type AAT protein. One embodiment may provide a human recombinant AAT protein comprising a polypeptide sequence having about 80%, about 85%, about 90%, or about 95% sequence identity to SEQ ID NO:1. A recombinant AAT protein of the embodiment may comprise a sequence having at least one mutation at position 51, 351, or 358 of the wild-type AAT sequence (SEQ ID NO:1). A further embodiment may provide for a human recombinant AAT protein comprising a polypeptide sequence having a SEQ ID NO:1 mutation, wherein the mutation comprises at least one of: a phenylalanine to a leucine at position 51 (F51L), a methionine to valine mutation at position 351 (M351V), or a methionine to valine mutation at position 358 (M358V). For example, a human recombinant AAT protein including variants thereof may have a single mutation from phenylalanine to leucine at position 51 (F51L) of the wild type AAT, where the variant recombinant AAT has improved thermostability over that of the wild type AAT. (Kwon K S, et al. JBC, 269(13):9627-9631, 1994; Kim J, et al. JBC, 270(15):8597-8601, 1995). Additional recombinant AAT proteins and variants thereof may include single or double mutant forms of AAT, such as but not limited to methionine to valine mutations at positions 351 and 358 (M351V; M358V). (Taggart C, et al. JBC, 275(35):27258-65, 2000). A further embodiment may include a recombinant AAT protein or variants thereof having a single mutation, a double mutation, or multiple mutations, including but not limited to a triple mutation comprising F51L, M351V, and M358V. (Zhu W, et al. FEBS Open Bio. 8(10):1711-1721, 2018; eCollection 2018 October) Yet another embodiment may provide a human recombinant AAT protein, including variants thereof, comprising a polypeptide sequence having a SEQ ID NO:1 mutation, where in the mutation is a phenylalanine to a leucine at position 51 (F51L), a methionine to valine mutation at position 351 (M351V), or a methionine to valine mutation at position 358 (M358V), or any combinations thereof. Mutations that prevent oxidation of some methionine residues may decrease protease inhibitory activity, including for example, elastase and the like. Accordingly, variant recombinant AAT proteins having amino acid mutations that decrease protease inhibitory activity are useful in embodiments of the invention. Moreover, variant recombinant AAT proteins having any amino acid mutations that enhance thermal stability are also useful in embodiments of the invention, including any positioned at the hydrophobic core of AAT. Methods for producing a human recombinant AAT protein or variant thereof having thermal stability, as well as improved thermal stability over plasma-derived AAT, may be a useful type of recombinant AAT protein.

Another embodiment may be directed to human recombinant AAT proteins including variants thereof as produced by methods for producing the same, where the recombinant AAT proteins including variants thereof have mutations with resistance against oxidation. These mutations may be selected from cysteine, methionine, or any other amino acid that could be oxidized, or a plurality of amino acid mutations. Non-limiting examples of mutations that resist oxidation include methionine 351, methionine 358, or both as based on the wild-type AAT protein sequence. The methods of producing a human recombinant AAT protein or variant thereof may result in a recombinant AAT protein or variant thereof having increased thermal stability as compared to a plasma-derived AAT protein, where the mutation is from a phenylalanine at position 51 to a leucine. Further embodiments of the invention may be directed to methods of producing a human recombinant AAT protein including variants thereof having improved thermal stability and resistance against oxidation, where methionines susceptible to oxidation of the wild-type AAT protein are mutated, modified, or varied.

Yet another embodiment may be directed to methods of producing human recombinant AAT proteins including variants thereof, where they have increased activity, a circulation half-life in vivo or in patients greater than that of plasma-derived AAT, facilitates the application of the AAT protein in any of the routes of administration, or combinations thereof. The methods of producing human recombinant AAT proteins including variants thereof may, in additional embodiments, be grown in a cell culture medium essentially free of any animal-derived components, i.e., without any animal components or any contaminating human or non-human animal proteins, including those which might induce an immune response. In one embodiment, the producing a human recombinant AAT protein comprising a culturing step that occurs in a culture medium, and the culture medium contains less than about 5% (vol/vol), less than about 4% (vol/vol), less than about 3% (vol/vol), less than about 2% (vol/vol), or less than about 1% (vol/vol) of animal-derived components (human or non-human), or the culture medium essentially contains no animal-derived components. A further embodiment provides a method for producing a human recombinant AAT protein comprising a culturing step that occurs in a culture medium, where the culture medium may or may not comprise a human recombinant insulin.

Yet another embodiment may be directed to a human recombinant AAT protein, including variants thereof, comprising a polypeptide sequence having about at least about 95% sequence identity to SEQ ID NO:1, about or at least about 97% sequence identity to SEQ ID NO:1, about or at least about 98% sequence identity to SEQ ID NO:1, about or at least about 99% sequence identity to SEQ ID NO:1. These recombinant human AAT proteins may be produced by any of the methods of producing a human recombinant AAT protein

In a further embodiment of the invention, a recombinant AAT derived from the modified CHO cells transfected and grown by methods described here may reduce inflammation. Another embodiment may be directed to an active recombinant AAT having more anti-inflammatory activity than that of a plasma-derived AAT, where, for example, the active recombinant AAT inhibits interleukin-6 (IL-6) and tumor necrosis factor (TNF) expression or release upon exposure of peripheral blood mononuclear cell (PBMC) to lipopolysaccharide (LPS). A further embodiment may be directed to an active recombinant AAT that protects against TNF-alpha- or endotoxin-induced disease or death. In yet another embodiment, an active recombinant AAT may exert anti-inflammatory and immune modulatory activities, but also anti-apoptotic activities. For example, recombinant AAT derived from the modified CHO cells described here may inhibit acetaminophen- or paracetamol-induced liver cell apoptosis that may typically be found in acute liver failure in mammals. A further embodiment may be directed to an active recombinant AAT having more anti-apoptotic activity than that of a plasma-derived AAT, where, for example, the active recombinant AAT inhibits liver injury induced by acetaminophen.

Another embodiment may be directed to the production of recombinant AAT preparation with human-like glycosylation and with a higher purity and quality than plasma-derived AAT products. A further embodiment encompasses the production of recombinant AAT preparations made in mammalian host cells that grow to very high density and that produce very high levels of recombinant AAT, thus allowing for commercially viable manufacture of these AAT preparations. In yet another embodiment, production of recombinant AAT preparations made in high-yielding CHO host cells as described here, and without any animal origin-derived components thus assuring a higher safety profile than human plasma-derived AAT.

### Recombinant AAT Compositions and Treatments Therewith

Embodiments described here provide for administration of compositions to subjects suffering from a deficiency of AAT, a biologically compatible form, which may also be a therapeutic composition, suitable for administration in vivo. The biologically compatible form comprises an active recombinant AAT that may be administered in which any toxic effects are outweighed by the therapeutic effects of the active recombinant AAT protein. An amount effective, at dosages and for periods of time to achieve the desired result may be utilized when administrating a therapeutically effective amount of the therapeutic composition comprising a recombinant AAT protein, such as for example, a human recombinant AAT protein, as defined here. Factors such as the disease state, age, sex, and weight of a subject or individual who may receive or be administered the therapeutic composition comprising a recombinant AAT, and the ability of AAT to elicit a desired response in the individual, are contemplated and considered for a therapeutically active amount of an active. These factors may also be considered for dosage purposes and as well as regimens may be adjusted accordingly thus providing the most optimum therapeutic response.

One embodiment may be directed to compositions, including pharmaceutical compositions, comprising an active recombinant AAT, including variants thereof, described here in an appropriate pharmaceutically acceptable carrier, diluent, or medium, including but not limited to, water, saline, aqueous buffer, and the like, that are sufficiently sterile for administration (e.g., intravenous, subcutaneous, etc.). In some embodiments, hyaluronidase-based formulations may be a useful vehicle for an active recombinant AAT (e.g., herceptin). A subject suffering from an alpha 1-antitrypsin deficiency may be treated by administering to the subject an effective amount of a recombinant AAT protein, including variants thereof, and in one embodiment, a human recombinant AAT protein, where the amount is effective to ameliorate or reduce the alpha 1-antitrypsin deficiency in the subject, thereby treating the subject and increasing the alpha 1-antitrypsin plasma level in the subject to a level such that the subject no longer suffers from an AAT deficiency. A further embodiment provides for a method of treating a subject suffering from an AAT deficiency by administering a human recombinant AAT protein, including variants thereof, or a composition comprising a human recombinant AAT protein including variants thereof, in an amount effective to ameliorate, improve, or reduce the AAT deficiency in the subject, thereby treating the subject. Another embodiment may be directed to a method of treating a subject suffering from a disease that results in protease-induced tissue damage, comprising administering to the subject an effective amount of a human recombinant AAT protein or composition comprising the human recombinant AAT protein to ameliorate the protease-induced tissue damage in the subject, thereby treating the subject.

Dosage formulations, dosage amounts, and routes of administrating such dosage formulations may vary depending on the nature and severity of the condition to be treated, the age and weight of the subject, etc. A dose of about 60 mg/kg of body weight administered, for example, once weekly via intravenous infusion may be a desired augmentation protocol using the recombinant AAT in subjects, for example, AAT-deficient patients or those in need of AAT. Alternatively, the dose may be increased or decreased based on the level to provide the best benefit for the subject suffering from an AAT deficiency. In another embodiment, dosing every two weeks at 120 mg/kg, dosing every third week at 180 mg/kg, or monthly dosing at 250 mg/kg may also be administered to the subject in need thereof. Compositions of the invention comprising an effective amount of a recombinant AAT or variant thereof may be administered by any suitable route to humans or non-human animals as deemed appropriate by a physician. It will be appreciated that the amount of the recombinant AAT or variant thereof according to this invention to be administered to the patient and required for use in treatment or prophylaxis according to the present invention will vary with the route of administration, the nature and severity of the condition for which treatment or prophylaxis is required, the age, weight, and condition of the patient, and will be ultimately at the discretion of the attendant physician. In general, however, a useful amount is such that by the administration of the pharmaceutical composition to the patient to be treated is improved to levels of a person who is not affected by an AAT deficiency. For example, a pharmaceutical composition and pharmaceutically acceptable carrier, diluent, or medium comprising a recombinant AAT or variant thereof as described here may be administered to a subject or patient suffering from an AAT deficiency by any appropriate route in an amount sufficient to increase AAT plasma levels of the AAT deficient subject to those AAT plasma levels of a healthy subject, i.e., about 1 g/L to about 2 g/L or greater. Since the recombinant AAT or variant AAT of the embodiments described here may have a longer half-life, weekly intravenous administrations may not be necessary. However, subjects or individuals may be monitored and dosages or regiments may be altered in order to provide the best outcome for the subject. The infusion rate may also vary; however, 0.08 ml/kg per minute has been a well-established rate that may be useful when administrating compositions comprising the recombinant AAT, including variants thereof.

Another embodiment may be directed to compositions including pharmaceutical compositions and pharmaceutically acceptable carriers, diluents, or mediums, where the compositions may contain the recombinant AAT including variants described here, where the compositions can be used externally (i.e., topically) and internally (i.e., by injections, infusions, inhalations) for diseases or conditions that cause an AAT deficiency, inflammation, protease-induced tissue damage, and the like, and may be administered to a subject in need thereof, by for example parenteral administration, including but not limited to, intravenously, intracardiacally, intracoronarily, intramuscularly, subcutaneously, by inhalation, bronchial/tracheal instillation, dermally, intradermally, transdermally, intramucosally, transmucosally, intravaginally, topically, intranasally, rectally, and the like, and combinations thereof. Administration may also occur in tissues and cavities by a route including but not limited to intraperitoneally, intrapleurally, intrathecally, intraarterially, parenterally, and the like, and combinations thereof. Intramucosal administration may occur via mucous membranes, such as but not limited to, lips, mouth, nasal passages, middle ear, eustachian tube, the lining of the digestive tract, the lining of the urogenital tract (including the urethra and vagina), the lining of the respiratory tract, and eyes (including conjunctival membranes), which may include topical application as well as, for example, intravitreal injection. Depending on the route of administration, the active recombinant AAT including variants thereof may be coated in a material to protect the compound from the degradation by enzymes, acids and other natural conditions that may inactivate the recombinant AAT. In one embodiment, the active recombinant AAT, including human recombinant AAT, variants thereof, or compositions comprising the recombinant AAT thereof, may be administered intravenously. In another embodiment, the active recombinant AAT, including human recombinant AAT, variants thereof, or compositions comprising the recombinant AAT thereof, may be administered intranasally, or by direct inhalation into the lungs. In yet a further embodiment, the active recombinant AAT, including human recombinant AAT, variants thereof, or compositions comprising the recombinant AAT thereof, may be administered topically.

In an embodiment of the invention, the active, recombinant AAT protein including variants thereof may be prepared in a composition for topical administration. Non-limiting examples of compositions comprising the recombinant AAT, including variants thereof, derived from the modified CHO cells described here include a solution, a spray, a lotion, a gel, a cream, a balm, a paste, or an ointment.

In another embodiment of the invention, subjects with a deficiency of AAT may be treated with the recombinant AAT protein including variants thereof described herein. Another embodiment may be directed to the treatment of diseases or conditions that are impacted by inflammation, immune reactions, or apoptosis, where the recombinant AAT protein including variants thereof inhibits inflammation, immune responses, and apoptosis.

The diseases to be treated with recombinant AAT preparations, including variants thereof, obtained from high-level producing mammalian cells cultivated in bioreactors—as demonstrated here—are to be selected from the group of diseases that are induced by any occurrence of reduced AAT activity in a given subject, and as they are presented by patients who suffer from, for example, functional AAT deficiency, from hepatic cirrhosis, from cystic fibrosis, from inflammatory skin diseases, from diabetes-induced lack of wound healing, protease-induced tissue damage, and others.

The recombinant AAT, including variants thereof, described here may also be used in treating subjects suffering from protease-induced tissue damage. There are a variety of underlying diseases that may manifest as protease-induced tissue damage. Non-limiting examples of these diseases include those that may be caused by any malignant disease, including but not limited to cancer (e.g., malignancies of epithelial origin and/or mesenchymal origin, sarcoma, malignant pleural mesothelioma), neurodegenerative disorders, and inflammatory and cardiovascular diseases. The diseases may also include those caused by inflammation of an undefined or unknown origin.

A further embodiment may be directed to treating a subject suffering from an AAT deficiency or protease-induced tissue damage by administering human recombinant AAT protein, including variants thereof, or compositions comprising human recombinant AAT protein including variants thereof and pharmaceutically acceptable carriers, diluents, or mediums, in tissues and cavities by a route of administration selected from any of: subcutaneously, intramuscularly, intraperitoneally, intrapleurally, intrathecally, intradermally, transdermally, intravenously, intraarterially parenterally, intramucosally, topically, inhalation, and the like, and combinations thereof.

Alpha 1 antitrypsin therapy may provide additional applications beyond anti-inflammatory actions and serine protease inhibition. These may include, in some embodiments, immunomodulation and anti-apoptosis through caspase inhibition such as but not limited to caspase-1, caspase-3, etc. These effects may be present or retained even if alpha-1 antitrypsin were to lose its anti-protease effect. See, e.g., Wanner A. (2016) Alpha-1 Antitrypsin as a Therapeutic Agent for Conditions not Associated with Alpha-1 Antitrypsin Deficiency. In: Wanner A., Sandhaus R. (eds) Alpha-1 Antitrypsin. Respiratory Medicine. Humana Press, Cham; Siebers K, et al. “Alpha-1 Antitrypsin Inhibits ATP-Mediated Release of Interleukin-1β via CD36 and Nicotinic Acetylcholine Receptors.” Front Immunol. April 25, 9:877, 2018, both of which are incorporated herein by reference in their entirety.

A further embodiment may be directed to treating a subject suffering from a destruction of pancreatic cells, due, for example, to an autoimmune disease, such as in children and adolescents and adults who suffer from diabetic disease (e.g., diabetes type I, maturity onset diabetes of the young (MODY)). An elevated level of circulating AAT in these patients, following injected human recombinant AAT, may prevent the destruction of beta cells and reverse the effects of hyperglycemia.

Another application for recombinant AAT may be in organ transplantation and preservation, which as a result may utilize safer and less dangerous techniques than common classical methods. Combination therapies with immunosuppressants for patients including those who subsequently become immune deficient and accumulate mostly potentiated toxicities in order to address graft dysfunction. Graft-versus-host disease oftentimes is a complication associated with stem cell transplantations. Perfusion solutions used during organ harvesting may be treated with recombinant AAT, for example, in Perfadex® for lung transplantations. Recombinant AAT may also be used during organ preservation or storage, and as a result, there may be an increase in living and dead donor pools, which may be a significant limiting factor for organ transplantation. Factors may include longer storage times, better organ protection, and the like such that less harm falls on the transplant patient including but not limited to injuries during transplantation, to the organ itself, the patient (e.g., brain damage), and fewer episodes of long-term graft failures or rejections.

In other embodiments, recombinant AAT may be of relevance for tissue protection in severe and vital non-organ transplantation diseases with huge components of ischemia-reperfusion injury, such as myocardial infarction, stroke, and the like. (See, e.g., Abouzaki N A, et al. Inhibiting the Inflammatory Injury After Myocardial Ischemia Reperfusion With Plasma-Derived Alpha-1 Antitrypsin: A Post Hoc Analysis of the VCU-α1RT Study. J Cardiovasc Pharmacol. 2018 June; 71(6):375-379; Mauro A G, et al. Preclinical Translational Study of the Cardioprotective Effects of Plasma-Derived Alpha-1 Anti-trypsin in Acute Myocardial Infarction. J Cardiovasc Pharmacol. 2017 May; 69(5):273-278; Toldo S, et al. Recombinant Human Alpha-1 Antitrypsin-Fc Fusion Protein Reduces Mouse Myocardial Inflammatory Injury After Ischemia-Reperfusion Independent of Elastase Inhibition. J Cardiovasc Pharmacol. 2016 July; 68(1):27-32; Toldo S, et al. Alpha-1 antitrypsin inhibits caspase-1 and protects from acute myocardial ischemia-reperfusion injury. J Mol Cell Cardiol. 2011 August; 51(2):244-51; Mollnes T E, et al. Acute phase reactants and complement activation in patients with acute myocardial infarction. Complement. 1988; 5(1):33-9, all of which are incorporated by reference in their entireties for the teachings of the use of recombinant AAT therapies.)

A further use of recombinant AAT is the use in hemolytic anaemia, sepsis, and other related diseases, to prevent or reduce damaging effects of free-heme release, particularly during hemolysis of red blood cells. Moreover, recombinant AAT may play an important role in graft-versus-host disease. (See, e.g., Janciauskiene, Sabina, and Tobias Welte. “Future directions: diagnostic approaches and therapy with AAT.” Alpha-1 Antitrypsin Deficiency 85 (2019): 159; Geiger S, et al. Alpha-1 Antitrypsin-Expressing Mesenchymal Stromal Cells Confer a Long-Term Survival Benefit in a Mouse Model of Lethal GvHD. Mol Ther. 2019 Aug. 7; 27(8):1436-1451; Thangavelu G, Blazar BR. Achievement of Tolerance Induction to Prevent Acute Graft-vs.-Host Disease. Front Immunol. 2019 Mar. 6; 10:309; Baranovski B M, et al. Alpha-1 Antitrypsin Substitution for Extrapulmonary Conditions in Alpha-1 Antitrypsin Deficient Patients. Chronic Obstr Pulm Dis. 2018 Sep. 19; 5(4):267-276; Magenau J M, et al. α1-Antitrypsin infusion for treatment of steroid-resistant acute graft-versus-host disease. Blood. 2018 Mar. 22; 131(12):1372-1379; Jerkins J H, et al. Alpha-1-antitrypsin for the treatment of steroid-refractory acute gastrointestinal graft-versus-host disease. Am J Hematol. 2017 October; 92(10):E610-E611; Lee S, et al. IL-32-induced Inflammatory Cytokines Are Selectively Suppressed by α1-antitrypsin in Mouse Bone Marrow Cells. Immune Netw. 2017 April; 17(2):116-120; Gerner R R, et al. Treatment With α-1-Antitrypsin for Steroid-Refractory Acute Intestinal Graft-Versus-Host Disease: A Report of 2 Cases. Transplantation. 2016 December; 100(12):e158-e159; Marcondes A M, et al. Response of Steroid-Refractory Acute GVHD to α1-Antitrypsin. Biol Blood Marrow Transplant. 2016 September; 22(9):1596-1601; Kekre N, Antin J H. Emerging drugs for graft-versus-host disease. Expert Opin Emerg Drugs. 2016 June; 21(2):209-18; Lior Y, et al. Therapeutic compositions and uses of alpha1-antitrypsin: a patent review (2012-2015). Expert Opin Ther Pat. 2016 May; 26(5):581-9, all of which are incorporated herein by reference for their teachings on the uses of AAT therapy).

A central role of disease burden may be caused by viral disease (e.g., in animals, of animal origin, with potential to spread to humans). The influenza A virus (and of note is that influenza infection has become the most important infectious disease of the world concerning morbidity, mortality and costs), coronaviruses with their specific epidemic or pandemic dangers and challenges, and also human immunodeficiency virus type 1 (HIV-1) use host serine proteases for cell entry and subsequent infection. Alpha-1 antitrypsin levels may play a role in disease spread and evolution (including viral spread reduction, inflammation modulation and reduction, and cell death prevention), and thus potentially may also be useful in augmentation therapy. (See, e.g., Wanner A. Alpha-1 antitrypsin as a therapeutic agent for conditions not associated with alpha-1 antitrtrypsin deficiency. In: Wanner A, Sandhaus R. S. (eds): Alpha-an antrypsin. Role in health and disease. Humana Press 2016; Springer International Publishing, Cham, Heidelberg, N.Y., Dordrecht, London, pp. 141-55, which is incorporated herein by reference in its entirety.)

Moreover, AAT may be useful in the treatment of Cystic Fibrosis and in Chronic Obstructive Pulmonary disease. Alpha-1 antitrypsin repletion may be of therapeutic potential, with respect to raising its level not to normal, but beyond normal levels. As Alpha-1 antitrypsin is a potent inhibitor of neutrophil elastase, aiming ultimately at losing the ability for progressive lung remodelling and destruction in cystic fibrosis. Furthermore, as neutrophil-dependent and neutrophil-independent inflammation as well as cell death by apoptosis are central, alpha-1 antitrypsin may be used in methods for tissue protection. (See, e.g., McElvaney N G. Alpha-1 Antitrypsin Therapy in Cystic Fibrosis and the Lung Disease Associated with Alpha-1 Antitrypsin Deficiency. Ann Am Thorac Soc. 2016 April; 13 Suppl 2:S191-6; Janciauskiene S, Welte T. Future directions: diagnostic approaches and therapy with AAT. In: Strand P, Prantyl M L, Bals R, eds. al-antitrypsin deficiency (ERS monograph). Sheffield, European Respiratory Society, 2019: pp. 158-78; Wanner A. Alpha-1 antitrypsin as a therapeutic agent for conditions not associated with alpha-1 antitrtrypsin deficiency. In: Wanner A, Sandhaus R. S. (eds): Alpha-an antrypsin. Role in health and disease. Humana Press 2016; Springer International Publishing, Cham, Heidelberg, N.Y., Dordrecht, London, pp. 141-55, all of which are incorporated herein by reference).

Yet another embodiment of the invention may be directed to administration of the active, recombinant AAT including variants thereof to individuals suffering from chronic obstructive pulmonary disease (COPD) or related diseases or conditions, including but not limited to liver disease, respiratory disease, and the like, where there is an AAT deficiency caused by a lack or deficiency of circulating AAT or an accumulation of AAT in the liver because it is not secreted properly. Non-limiting examples of diseases, conditions, or disorders that may be treated with the recombinant AAT including variants thereof include: emphysema, cirrhosis, liver failure, COPD, pneumothorax, asthma, granulomatosis with polyangiitis, pancreatitis, bronchiectasis, autoimmune hepatitis, and any malignant disease, including but not limited to some cancers (e.g., malignancies of epithelial origin and/or mesenchymal origin, sarcoma, malignant pleural mesothelioma), and those cancers of, for example, the lungs, liver, and bladder.

One embodiment may be directed to augmentation or replacement therapy using the active, glycosylated recombinant AAT protein including variants thereof by infusing the protein intravenously into a subject suffering from an AAT deficiency. The administration may follow conventional methods except that instead of infusing plasma-derived AAT protein, embodiments of the invention may be directed to administration using the active, recombinant AAT protein including variants thereof derived from modified CHO cells as produced by the methods and as described here.

A further embodiment may be directed to administration of the recombinant AAT protein CHO-derived recombinant AAT efficiently through skin or mucosa to access, for example, the lungs, the respiratory system, the circulatory system, and the like. In one embodiment, the recombinant AAT may be inhaled and used as a topical treatment of, for example, lung emphysema induced by AAT-deficiency. Recombinant AAT may traverse layers of skin in a fashion that is more efficient and improved over plasma-derived AAT.

The problem is furthermore solved by embodiments of the present invention that provide a method of treating a disease, wherein the recombinant AAT made in cultivated mammalian cells, is produced in quantities and with a quality useful for ameliorating or improving the levels of AAT in AAT deficient subjects or those subjects who are affected by diseases, disorders, or conditions associated with AAT and are in need thereof.

Immune therapy has taken an important place in different cancer types and has extensively changed the prognosis in some malignant diseases, including but not limited to cancers (e.g., malignancies of epithelial origin and/or mesenchymal origin, sarcoma, malignant pleural mesothelioma). A clinical obstacle under such treatment is the occurrence of autoimmune diseases due to the potent curbing of autoimmunity. AAT may be used in autoimmune disease treatment in some embodiments.

### EXAMPLES

The following examples illustrate specific aspects of the instant description. The examples should not be construed as limiting, as the examples merely provide a specific understanding and practice of the embodiments and their various aspects.

### Example 1: Alpha 1-Antitrypsin (AAT) Protein Sequence

The most common allele of the AAT gene (i.e., the M-allele) in the form of a synthetised CHO codon optimized DNA was cloned into a high-performance expression vector. The desired protein of interest (FIG. 1A without leader sequence; FIGS. 1B-1D with leader sequences) encoded by the Gene of Interest (GOI) was expressed from the pXLG-6-AAT expression vector. See, FIGS. 2A-2C and FIG. 3. The sequence of the wild type AAT gene with 394 amino acids is highly conserved in primates, with only one amino acid difference in Chimpanzees in comparison to the M-allele of humans. In spite of this, in the human population, more than 50 allele variants are present, many of them contributing to or being the reason for disease. The M-allele sequence can be found in Long G L, et al. (Biochemistry 23(21): 4828-4837, 1984), but is also available from the Human Genome Sequencing platforms (see appendix). The AAT protein exhibits three N-linked glycosylation sites.

The alpha 1-antitrypsin (AAT) sequence used is identical to the sequence published in Long G L, et al. (Biochemistry 23(21):4828-4837, 1984); however, the leader sequence (i.e., secretory signal peptide sequence) of the original sequence is replaced with the leader sequence of a human heavy chain IgG1 sequence. According to a signal peptide cleavage program, the same mature polypeptide would be made in CHO cells, starting with the expected first three amino-terminal amino acids being Glutamic Acid (E), Aspartic Acid (D), and Proline (P). The SERPINA1 gene encodes the alpha 1-antitrypsin (AAT) protein that is based on the gene sequence represented by the M-allele, which is considered to be the most common and functional human AAT. The secreted full-length wild type AAT amino acid sequence has 394 amino acids, as published in Long G L, et al. 1984.

The sequence, including the human heavy chain IgG1 leader sequence (in bold and underlined) is provided in FIG. 1C as the amino acid sequence of the recombinant human Alpha-1 antitrypsin protein (M).

### Example 2: Host Cell Expression System for Alpha 1-Antitrypsin

The modified CHO cells described here (i.e., fast growing, high protein yielding, high density robustness, scalability under suspension culture, etc.) were developed for bioreactor use, i.e., large-scale manufacturing, by modifying and selecting a specific Chinese hamster ovary (CHO) cell line that was originally generated in an academic laboratory (Puck T T, et al. J. Exptl. Med. 108(6):845-956, 1958) that had been subsequently provided to numerous researchers, including university groups, over the decades. CHO cells are known to change their genetic make-up rapidly and to adapt to various culture conditions in a manner that is very similar to cancer cells in humans or animals. The inventive cells were derived from a non-recombinant cell host, extensively optimized for rapid, robust growth under suspension culture in animal component-free media. These phenotypic features were inherited after transfection to the recombinant cells that express AAT, and among other characteristics allowed these cells to grow to a density of greater than 20 million cells/mL and a growth rate resulting in a doubling rate of less the 20 hours/cell doubling, yielding over about 5 g/L of ATT or over about 6 g/L, or over 7 g/L or over 8 g/L of AAT, demonstrating the robustness and productivity in large-scale manufacturing, while growing in an animal component-free media. Clonal CHO cell lines expressing recombinant human AAT were successfully established using a highly efficient transposon-based gene transfer technology described here. The convoluted history of CHO cells and their uncertain genetic makeup was described in a publication. (Wurm, F. M. Processes 1(3):296-311, 2013). The literature also reported levels of AAT expression in CHO cells only as 100 μg/L per day (Paterson T, et al. Applied Microbiol. Biotechnol. 40:691-698, 1994) and more recently at about 1 g/L as a final yield (Lalone M-E et al. J. Biotechn. 307 (2020) 87-97, 2019), but in the human retinal tissue PerC6 cell line AAT was expressed in amounts just over 2.5 g/L as a final yield (Ross D, et al. J. Biotech. 162(2-3):262-273, 2012).

### Example 3: Expression Vector System for Transfection

A two-vector co-transfection approach was used for the transfection of CHO cells with the desired AAT protein of interest. The desired AAT protein is encoded by the gene of interest (GOI)-vector, pXLG-6-AAT expression vector (ExcellGene S.A.; FIG. 2A, FIG. 2B, FIG. 2C, FIG. 2D). FIG. 3 shows the pXLG-6 vector map into which the AAT gene was cloned within the multi-cloning site (MCS). A second plasmid vector, pXLG-5 (ExcellGene S.A.), encoded for the PiggyBac transposase (mPBase) (SEQ ID NO:10) (FIG. 4A, FIG. 4B) was used in the two-vector co-transfection approach.

The most common human allele of the AAT gene (i.e., the M-allele) in the form of an intron-free DNA was cloned into a high-performance expression vector, for example, the pXLG6 expression vector. The sequence of the wild type AAT gene with 394 amino acids is highly conserved in primates, with only one amino acid difference in Chimpanzees in comparison to the M-allele of humans. In spite of this, in the human population, more than 50 allele variants are present, many of them contributing to or being the reason for disease. The M-allele sequence can be found in Long G L, et al. Biochemistry 1984, 23, 4828-4837, but is also readily available from the Human Genome Sequence platforms. The wild type human AAT protein exhibits three N-linked glycosylation sites. The recombinant AAT of embodiments of the invention described here may have additional glycosylation sites.

### Example 4: Transfection and Selection of Recombinant AAT-Expressing CHO Cells

Non-recombinant host cells having the desired phenotype for rapid growth while in an animal component-free media were used in generating a master cell bank (MCB), which was verified as being Chinese hamster ovary cells. The described efficient transposon-based gene transfer technology occurs by co-transfecting the host cells with a donor vector comprising the AAT gene sequence and a second transposase expressing nucleic acid that mediates the insertion of the AAT gene into the genome of the host cells with the help of a transposase encoded by the transposase gene. Cells derived from a seed train culture under suspension culture in ProCHO5 medium (Lonza) (established from the aforementioned Master Cell Bank) were spun down by centrifugation and transfected strictly following instructions provided in the commercially provided transfection kit (CHO4Tx®; ExcellGene SA), providing 5 μg of the expression vector cocktail (comprising of the two aforementioned vectors) for a cell culture volume of 10 ml. Subsequently, transfected cells were cultured under suspension while shaking in a CO₂-controlled and humidified incubator-shaker at 180 rounds per minute and at 37° C. Each day, the cell culture was centrifuged and the sedimented cells, formed as a pellet in the container, were taken up in fresh pre-warmed medium provided by the transfection kit (CHO4Tx®), containing 50 μg/ml of puromycin for selection. The viability and cell number of the culture decreased for about 4 days after transfection, but then the cell population began to recover and both viability and cell number began to increase. After 10 days under selective conditions, a highly viable population of healthy growing cells was re-established. No further puromycin selection of the established culture was executed. This rapidly growing, recombinant population of cells was shown to express human recombinant AAT at high levels. This population of cells was considered a “pool” of recombinant AAT cells, representing a mixture of multiple and diverse genetic integration events of the AAT gene into the genome of the CHO cells.

The expanded recombinant cell population, i.e., the recombinant CHO-AAT pool, was used to generate a Research Cell Bank (RCB-P-rAAT; 10 vials). A vial of this population was thawed, expanded using Lonza ProCHO 5 medium, and single cell cloned and re-cloned a second time using a limiting dilution approach. This approach assured a clonal origin of the emerging cell populations with a probability of greater than 98%. The Research Cell Bank “RCB-P 03-rAAT” was used for the twice limiting dilution approach of single cells and delivered 72 highly productive clonally-derived cell populations, five (5) of which were further studied in long-term cultures. These long-term cultures were derived from additional Research Cell Banks, generated with the clonally-derived cell populations, indicated as RCB-0 and RCB-1, respectively, and the corresponding clone name. The productivity of subcloned cells of these 5 cell lines (from the bank RCB-1) remained stable and the four (4) best performing clonally derived cell populations were designated as “clones 112, 423, 555 and 585” and frozen again as RCB-2 with an indication of the corresponding clone name.

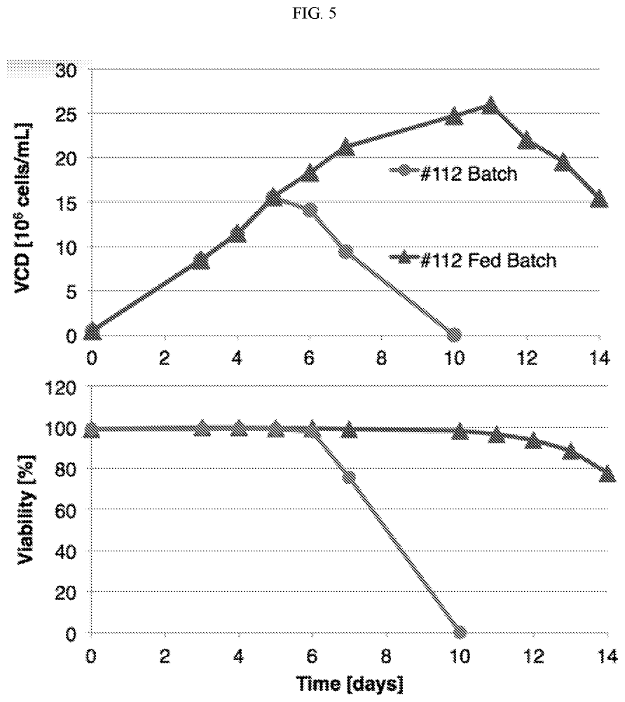

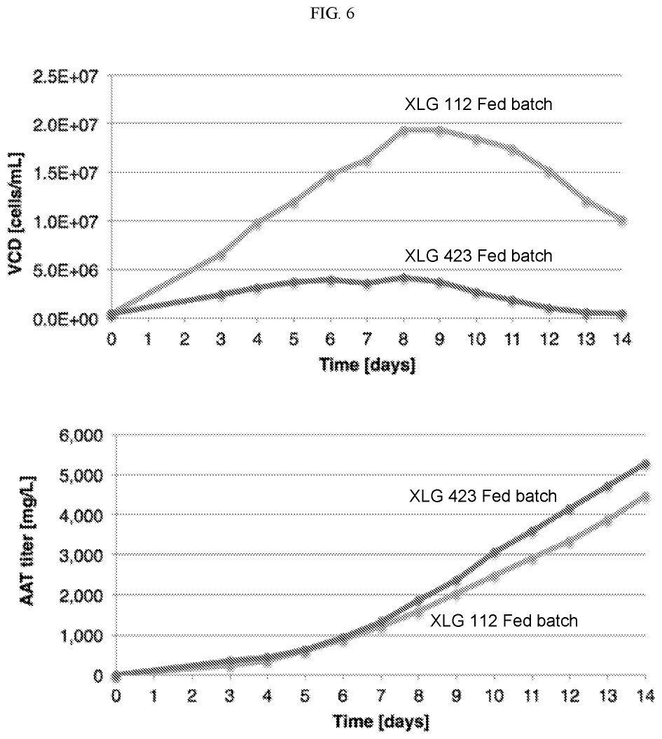

Two of these cell lines, XLG-AAT#112 and XLG-AAT#423, were derived from yet an additional Research Cell Bank RCB-3, when studied under batch and fed-batch conditions with a non-optimized process (FIGS. 5 and 6). Under these non-optimized cell culture production conditions these two clonally derived cell populations showed expression levels of 4 g/L and 5 g/L, respectively, after a 14-day process, which was significantly higher than the yields obtainable from human plasma (i.e., about 1-1.5 g/L).

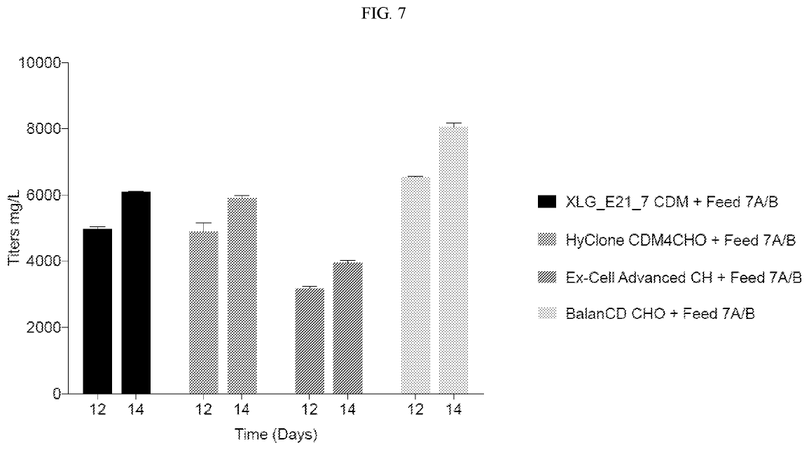

Another clonal cell line, XLG-AAT#30, was developed from transfection performed under similar conditions as described previously. The production conditions included the use of a diversity of media and feeds, without being optimized in any profound way, in 10 ml cultures maintained in 50 ml Orb Shake™ tubes. As can be seen in FIG. 7, under certain of these studied conditions, product titers of 6-8 g/L were obtained over 14 days. Following the schedule used in FIG. 9, at time points of 12 and 14 days, four different media conditions were compared (n=4). The black columns (far left) refer to the use of an ExcellGene medium (XLG_E21_7 CDM) in combination with Feeds 7A/7B (HyClone™ Cell Boost 7a Supplement (SH31026.01); HyClone™ Cell Boost 7b Supplement (SH31027.01); GE Healthcare Life Sciences); HyClone CDM4CHO with Feeds 7A/7B (GE Healthcare Life Sciences), Ex-Cell® Advanced™ CHO Fed-batch medium from Sigma-Aldrich, and BalanCD is a medium from Irvine.

### Example 5: Alpha 1-Antitrypsin (AAT) Protein Produced in Chinese Hamster Ovary (Cho) Cells

A culture of 10 ml of non-recombinant CHOExpress™ cells (ExcellGene SA) at a density of 1 million cells/ml were co-transfected with a highly efficient transposon-based gene transfer system comprising the plasmid vector pXLG6-AAT comprising the expression cassette for recombinant AAT and the plasmid vector pXLG5 comprising the piggyBac transposase for transposase-mediated gene integration. The non-recombinant CHOExpress™ cells can act as a high-performing production host system for large-scale manufacturing using, for example, a bioreactor with an efficient mixing system. The host cells were transformed with the nucleotide sequence encoding the human AAT of interest (SEQ ID NO:5), where the vector comprising the nucleotide sequence of interest was the pXLG6-AAT vector (SEQ ID NO:9), and a transformant (i.e. a clonally derived cell population) was isolated expressing the recombinant AAT protein (e.g., SEQ ID NO:1 or variants of SEQ ID NO:1). The clonally-derived cell lines were selected by single cell cloning and expansion. Among numerous others, five stable recombinant clonal CHO cell lines (XLG AAT#112, XLG AAT#275, XLG AAT#423, XLG AAT#555, XLG AAT#585) were banked and studied for further analysis.

The cell growth for each of the established cell lines were studied in simple fed-batch (FB or F.B.) processes, using chemically-defined medium (e.g., XLG_E21_7; ExcellGene S.A.) and a single addition of Feed A (ExcellGene S.A.), and demonstrated acceptable cell growth and maintained high cell viability levels for up to 14 days under these conditions (data not shown). The highest cell densities for certain cell lines, including but not limited to XLG 112 Fed-batch, reached peak growth on or about Day 9 achieving about 18-19×10⁶ cells/ml viable cell density (VCD) (FIG. 6). By Day 14, the cell density dropped to about 10×10⁶ cells/ml for XLG 112 Fed-batch.

AAT production in each of the five clonal CHO cell lines, i.e., AAT clonal Nos. 112, 275, 423, 555, and 585) (FIG. 8) under different culture conditions demonstrated that use of the medium (XLG_E21_7 CDM, ExcellGene S.A.) with an XLG feed (XLG FB; black column) was greatest at Day 14 compared to a commercially available, chemically defined medium (CDM) (e.g., PowerCHO™ 2 Serum-free CDM; LONZA; #BE12-771Q) or an XLG medium alone (gray column, e.g., XLG_E21_7 CDM without XLG feed; ExcellGene S.A.) at Day 6. All cell lines were compared in suspension cultures using a process involving a seed density of 0.5×10⁶ cells/ml, a production run time of 14 days, a temperature shift, and under fed-batch conditions with certain feeds (ExcellGene S.A.). The titers of the XLG AAT#112 CHO cell line cultured in an XLG media and feed process (FB) on Day 14 had about 4.5 g/L of AAT titer. Whereas, the same cell line on Day 6 in a commercially available CDM and XLG-medium without XLG feed (i.e., Batch process) had about 0.750 g/L and about 1.1 g/L of AAT titer, respectively (p<0.05). Recombinant AAT from different CHO cultures were analyzed by SDS-PAGE for protein expression under different fed-batch processes. The XLG AAT#112 clonal CHO cell line culture was repeatedly found to highly express AAT under various conditions.

Further investigation of the XLG AAT#112 clonal CHO cell line culture as cultured in 12 different fed-batch processes demonstrated that two particular fed batch processes (i.e., Conditions 1 and 3) delivered a titer of more than about 6 g/L AAT by Day 17 (FIG. 9). The fed-batch process under Conditions 1 and 3 entailed: a seed density of 0.5×10⁶ cells/ml in a CDM (e.g., XLG_E21_7; ExcellGene S.A.) and grown at temperature 37° C. for Day 0-Day 3, temperature 33° C. for Day 3-Day 17, and supplemented with Feeds 7a and 7b (HyClone™ Cell Boost; GE Healthcare Life Sciences) every other day (EOD) or every day (ED), respectively for Conditions 1 and 3, as indicated.

The table inset of FIG. 9 refers to chemically-defined medium feeds, 7a and 7b, as well as to temperature shifts executed during a fed-batch process. The 7a and 7b medium feeds are commercially available (HyClone™ Cell Boost 7a Supplement (SH31026.01); HyClone™ Cell Boost 7b Supplement (SH31027.01); GE Healthcare Life Sciences) and are given to the production process in certain volumes represented in a percent of the total culture volume (EOD: every second day, ED: every day). Temperatures of the production cultures are given over periods of time indicated as Day 0 (d00) to Day 3 (d03); Day 3 to Day 5 (d05) or Day 17 (d17); and Day 5 to Day 17 as indicated. The columns from the left to the right indicate the number of days, i.e., 7, 11, 14, and 17, respectively, when samples for product concentration in the culture were taken and analyzed for AAT titers.

In experiments designed for optimization of sialic acid (SA) content with the cell line XLG AAT#112, a high sialic acid (SA) content of about 5.5 moles of sialic acid/mole of AAT on Day 7 was observed. In contrast, commercially available plasma-derived AAT (Prolastin®, Grifols) was found to have 3.5 moles of sialic acid per mole of AAT. Thus, the XLG AAT#112 CHO cell line was found capable of producing a recombinant AAT with a sialic acid content of greater than about 55% and may be up to about 80% greater than that of plasma-derived AAT under certain conditions. Cells were grown in medium (e.g., ExcellGene SA) and the production was initiated at a seed density of 1×10⁶ cells/ml. The temperature was maintained at 37° C. from the start of production until Day 3 and then shifted to 33° C. until the end of production. The HyClone™ Cell Boost 7a and 7b feeds were used at a volume of 7.1% and 0.71%, respectively, and fed every other day, starting from Day 3 in accordance with Process Condition #1. While the 7a and 7b feeds were used at a volume of 3.6% and 0.36%, respectively, and fed every day, starting from Day 3 in accordance with Process Condition #3.

### Example 6: Recombinant AAT from Cho Cells Reduces Elastase Activity In Vitro

For quantification of the inhibitory capacity of elastase, recombinant AAT (recAAT) or plasma-derived AAT (plasma AAT) was pre-incubated with pancreatic elastase (E7885; Sigma Aldrich) (FIG. 11A-FIG. 11B). Thereafter, the remaining elastase activity was determined spectrophotometrically (FIG. 11C). In more detail, AAT was diluted to 0.08 mg/ml with assay buffer (0.1 M Tris buffer, pH 8) and 5 μl of diluted AAT was mixed with 5 μl of elastase (0.26 μM) in a total volume of 275 μl. After 5 minutes of incubation at 37° C., N-succinyl-Ala-Ala-Ala-p-nitroanilide substrate (S4760, Sigma Aldrich) was added and absorbance at 405 nm was measured for 3 min using an Infinite® M200 microplate reader (Tecan). A sample containing substrate and buffer alone was used for blank reduction and another sample with elastase, substrate, and buffer to determine 100% activity or 0% inhibition. All samples were analyzed in duplicate. (FIG. 11A). FIG. 11B shows the percent reduction of elastase activity with recombinant AAT and plasma-derived AAT in comparison to the AAT-free control. The complex-formation of AATs with elastase was demonstrated by running a 7.5% SDS-PAGE, stained with Coomassie Brilliant Blue R350, on samples which contained reaction mixes of elastase and AATs (FIG. 11D).

### Example 7: Recombinant AAT Reduces the Activity of Other Proteases, Such as Trypsin In Vitro

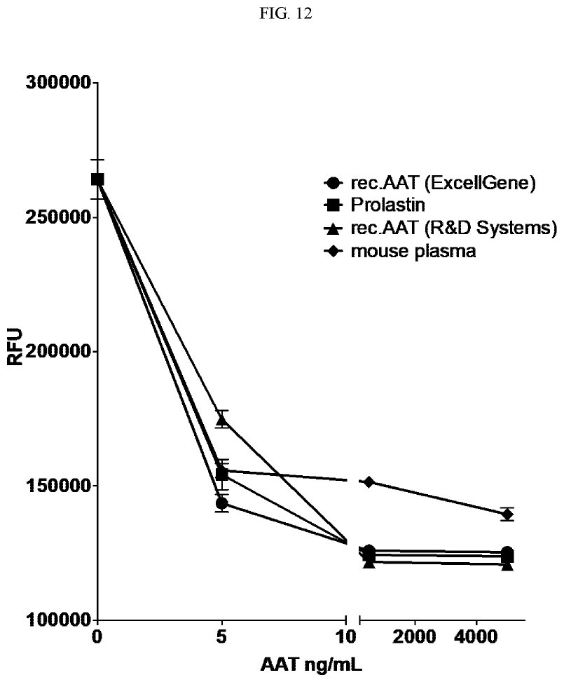

Trypsin is a widely used and powerful protease. Different concentrations of AAT from different origins, including the recombinant AAT and variants thereof, that are described and obtained using the methods disclosed here were added to a trypsin-containing buffer (0.25 μg/ml), together with a fluorogenic peptide (Mca-R-P-K-P-V-E-Nval-W-R-K(Dnp)-NH₂; SEQ ID NO:17) as a substrate. At a concentration of 5 ng/ml, the recombinant AAT described and obtained by the methods presented herein (ExcellGene) drastically reduced trypsin activity in a similar manner as a plasma-derived AAT (Prolastin), another recombinant AAT (R&D systems), or mouse plasma diluted from a sample that contained about 1.2 g/L mouse AAT (FIG. 12). Experiments were performed in triplicate. The horizontal lines near some of the symbols indicate the variation from the mean value.

### Example 8: Recombinant AAT Reduces Endotoxin- (or Liposaccharide-) Induced TNF-Alpha Release

Adherent human peripheral blood mononuclear cells (PBMCs) were treated for 4 hours with lipopolysaccharide (LPS, 1 μg/ml). Afterwards, LPS was removed by washing three (3) times, and subsequently various amounts of recombinant AAT (ranging from 0 mg/ml to 1 mg/ml) were added for 10 hours. Supernatants were analyzed by a TNF-α specific immunoassay using the Human TNF-α Quantikine® ELISA designed to measure human TNF-α in cell culture supernatants, serum, and plasma (R&D Systems; Minneapolis, Minn., USA). The results are shown in FIG. 13. The duplicate run samples show that at 0.5 mg/ml to 1 mg/ml of recombinant AAT, there was a decrease in TNF-alpha release by 1 μg/ml lipopolysaccharide.

In FIG. 14A-FIG. 14B, peripheral blood mononuclear cells (PBMCs) were incubated alone, with lipopolysaccharide (LPS) (1 μg/ml), or with both LPS and recombinant AAT (1 mg/ml) for 10 hours. mRNA (TNF-α (FIG. 14A) and IL-6 (FIG. 14B)) was isolated and analyzed for gene expression. Total RNA was prepared using RNeasy® Micro Kit (QIAGEN). For cDNA synthesis, 1 μg of total RNA was transcribed using a high-capacity cDNA reverse transcription kit (Applied Biosystems, Thermo Fisher Scientific). mRNA levels of selected genes were determined using TaqMan® Gene Expression Assays (Applied Biosystems, Life Technologies) on a StepOnePlus™ Real-Time PCR System (Applied Biosystems) as compared to the housekeeping gene, hypoxanthine guanine phosphoribosyl transferase (HPRT).

### Example 9: AAT-Treated Skin In Vitro Analysis

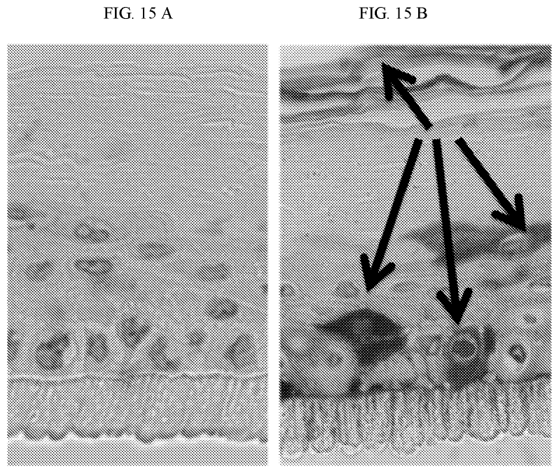

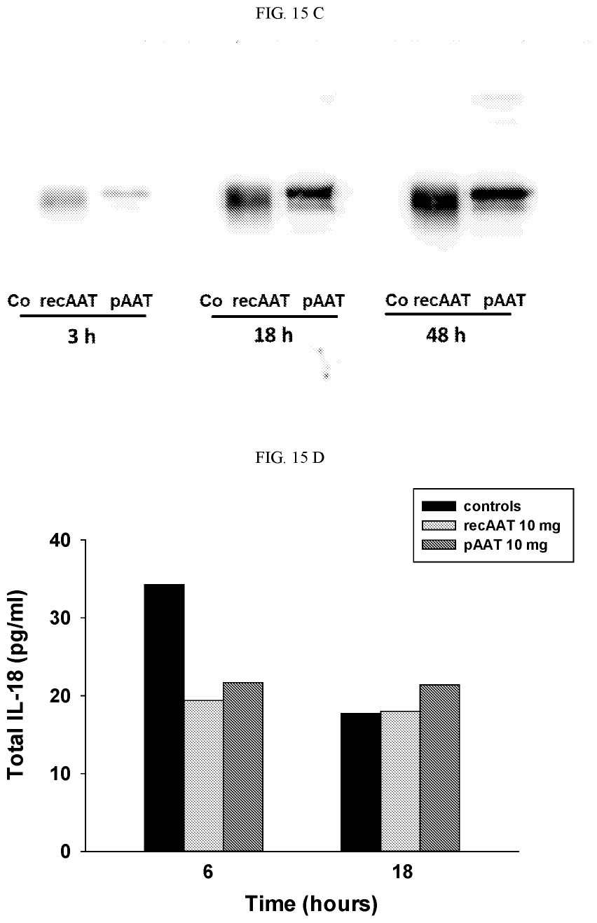

AAT was applied (10 mg recAAT) to the “external” layer of a human skin model (epiCS®; CellSystems) and incubated for several hours (18 hours) as described here. Subsequently, AAT was identified below the skin layers through a Western Blot approach identifying AATs using specific antibodies. Untreated epiCS® skin stained with rabbit polyclonal anti-human AAT antibody (1:5000; DAKO; Denmark) showed that epiCS® had no staining for AAT (FIG. 15A); whereas, epiCS® skin treated with recAAT (10 mg) for 18 hours and then stained demonstrated positive staining for AAT (grey darker areas and arrows, FIG. 15B). Supernatant analysis of epiCS® culture supernatant treated with recombinant AAT and plasma AAT by Western blot showed increasing AAT expression over 3 hours, 18 hours, and 48 hours. (FIG. 15C). FIG. 15D shows the results of an ELISA (R&D Systems, USA) analysis of total levels of IL-18, a pro-inflammatory cytokine and a marker of skin irritation, in epiCS® skin supernatant. Skin irritation was not induced by 10 mg recAAT (light grey) and 10 mg plasma AAT (dark grey; pAAT; Prolastin®). In fact, at 6 hours, both recAAT and pAAT showed an inhibitory effect on IL-18 release as opposed to control without AAT (black).

### Specific Embodiments

Non-limiting specific embodiments are described below each of which is considered to be within the present disclosure.

Specific embodiment 1) A method for producing a human recombinant alpha 1-antitrypsin (AAT) protein, comprising:
a) introducing into a host cell, an expression vector comprising a nucleic acid fragment encoding the human AAT protein;
b) culturing the host cell under conditions which allow for expression of the human recombinant AAT protein; and
c) isolating the human recombinant AAT protein from the cultured host cell, thereby producing the human recombinant AAT protein.

Specific embodiment 2) The method according to specific embodiment 1, wherein the nucleic acid fragment comprises a nucleic acid sequence encoding a human AAT CHO-cell codon-optimized sequence (and is driven by an optimized constitutive promoter).

Specific embodiment 3) The method according to specific embodiment 1 or specific embodiment 2, where the introducing step comprises co-transfecting the human recombinant AAT expression vector and an expression vector encoding a transposase.

Specific embodiment 4) The method according to specific embodiment 3, wherein the transposase is a piggyBac transposase.

Specific embodiment 5) The method according to any of specific embodiments 1-4, wherein the host cell is a eukaryotic cell.

Specific embodiment 6) The method according to any of specific embodiments 1-5, wherein the host cell is a Chinese hamster ovary (CHO) cell line.

Specific embodiment 7) The method according to specific embodiment 6, wherein the CHO cell line is a modified CHO cell line.

Specific embodiment 8) The method according to any of specific embodiments 1-7, wherein the culturing step occurs in a culture medium, and the culture medium contains less than about 5% (vol/vol) of animal-derived components.

Specific embodiment 9) The method according to any of specific embodiments 1-8, wherein the culturing step occurs in a culture medium, and the culture medium contains less than about 2% (vol/vol) of animal-derived components.

Specific embodiment 10) The method according to any of specific embodiments 1-9, wherein the culturing step occurs in a culture medium, and the culture medium consists of a chemically defined composition and does not contains a human recombinant insulin or any other protein.

Specific embodiment 11) The method according to any of specific embodiments 1-10, wherein the produced human recombinant AAT protein was in an amount of about 1 g/L to about 10 g/L of human recombinant AAT protein.

Specific embodiment 12) The method according to any of specific embodiments 1-11, wherein the produced human recombinant AAT protein was in an amount of about 2 g/L to about 6 g/L of human recombinant AAT protein.

Specific embodiment 13) The method according to any of specific embodiments 1-12, wherein the culturing step comprises:
selecting the host cell with the nucleic acid fragment expressing the human recombinant AAT protein, wherein the selected cells are clonally-derived cells expressing human recombinant AAT.

Specific embodiment 14) The method according to specific embodiment 13, wherein the selecting step comprises:
a) culturing the clonally-derived cells expressing human recombinant AAT in a culture medium;
b) feeding the clonally-derived cells expressing human recombinant AAT with at least one feed;
c) maintaining the culture medium at a cell culture temperature;
d) decreasing the cell culture temperature; and
e) culturing the clonally-derived cells at the decreased cell culture temperature, wherein the clonally-derived cells express the human recombinant AAT protein at a titer of about 1 g/L or greater.

Specific embodiment 15) The method according to any of specific embodiments 13-14, wherein the clonally-derived cells express human recombinant AAT protein at a titer of greater than about 4 g/L.

Specific embodiment 16) The method according to any of specific embodiments 13-15, wherein the clonally-derived cells express human recombinant AAT protein at a titer of greater than about 6 g/L.

Specific embodiment 17) The method according to any of specific embodiments 14-16, wherein the cell culture temperature ranges from about 35° C. to about 38° C.

Specific embodiment 18) The method according to any of specific embodiments 14-17, wherein the cell culture temperature is maintained from Day 0 to Day 3 or Day 0 to Day 5.

Specific embodiment 19) The method according to any of specific embodiments 14-18, wherein the decreased cell culture temperature ranges from about 25° C. to about 34° C.

Specific embodiment 20) The method according to any of specific embodiments 14-19, wherein the cell culture medium is at a decreased cell culture temperature from Day 3 to Day 17, Day 3 to Day 5, Day 5 to Day 17, or combinations thereof.

Specific embodiment 21) The method according to any of specific embodiments 14-20, wherein the at least one feed comprises a neutral feed.

Specific embodiment 22) The method according to specific embodiment 19, wherein the neutral feed is in a volume ranging from about 1% to about 8% of the total cell culture volume.

Specific embodiment 23) The method according to any one of specific embodiments 14-22, wherein the at least one feed comprises an alkaline feed.

Specific embodiment 24) The method according to specific embodiment 23, wherein the alkaline feed is in a volume ranging from about 0.1% to about 0.8% of the total cell culture volume.

Specific embodiment 25) The method according to any of specific embodiments 14-24, wherein the at least one feed comprises a neutral feed and an alkaline feed.

Specific embodiment 26) The method according to specific embodiment 25, wherein the alkaline feed is in an amount one-tenth ( 1/10) of an amount of a neutral feed.

Specific embodiment 27) The method according to any of specific embodiments 14-26, wherein the feeding step occurs every day.

Specific embodiment 28) The method according to any of specific embodiments 14-26, wherein the feeding step occurs every other day.

Specific embodiment 29) The method according to any of specific embodiments 1-28, wherein the culturing step comprises an osmolarity of the cell culture of about 550 mOsm/kg or greater.

Specific embodiment 30) The method according to any of specific embodiments 1-29, wherein the culturing step comprises an osmolarity of the cell culture of about 550 mOsm/kg or greater at Day 5 or later.

Specific embodiment 31) A method for producing a human recombinant alpha 1-antitrypsin (AAT) protein, comprising:
a) introducing into a eukaryotic host cell, a first nucleic acid sequence encoding a human AAT protein and at least an additional nucleic acid sequence encoding a transposase;
b) culturing the eukaryotic host cell under conditions which allow expression of the first nucleic acid sequence encoding a human AAT protein;
c) selecting the eukaryotic host cell with the nucleic acid fragment expressing a human AAT protein, wherein the selected cells are clonally-derived cells expressing human recombinant AAT protein; and
d) isolating the human recombinant AAT protein from the clonally-derived cells, thereby producing the human recombinant AAT protein.

Specific embodiment 32) The method according to specific embodiment 31, wherein the eukaryotic host cell is transformed with the nucleic acid sequence encoding a human recombinant AAT protein.

Specific embodiment 33) The method according to specific embodiment 31 or specific embodiment 32, wherein the step of isolating comprises purifying the human recombinant AAT protein.

Specific embodiment 34) The method according to specific embodiment 33, wherein the step of purifying is by at least one of: size exclusion chromatography, affinity chromatography, ion exchange chromatography, hydrophobic interaction chromatography, reverse phase chromatography, gel filtration, magnetic bead separation, selective precipitation, molecular weight-based membrane filtration or exclusion, buffer exchange, virus filtration, pH-based inactivation of viruses, and the like.

Specific embodiment 35) The method according to any of specific embodiments 1-34, wherein the isolated human recombinant protein has a purity of about or greater than about 95%.

Specific embodiment 36) The method according to any of specific embodiments 1-35, wherein the isolated human recombinant protein has a purity of about or greater than about 98%.

Specific embodiment 37) An expression vector, comprising: a nucleic acid fragment containing a nucleotide sequence encoding a human recombinant AAT protein, wherein the nucleic acid fragment is positioned in a multiple cloning site; an intron upstream of the nucleic acid fragment; a cytomegalovirus (CMV) promoter upstream of an intron; a 5′ Inverted Terminal Repeat (5′ ITR) upstream of the CMV promoter; a poly-adenosine tail signal sequence downstream of the nucleic acid fragment; a replication origin sequence downstream of the nucleic acid fragment; a selectable marker sequence downstream of the replication origin sequence; and a 3′ Inverted Terminal Repeat (3′ ITR) downstream of the selectable marker sequence.

Specific embodiment 38) The expression vector according to specific embodiment 37, wherein the selectable marker sequence is a puromycin resistance gene.

Specific embodiment 39) The expression vector according to any of specific embodiments 37-38, wherein the nucleic acid fragment and the selectable marker sequence are positioned in opposite reading frames and in between the 5′ ITR and the 3′ ITR.

Specific embodiment 40) The expression vector according to any of specific embodiments 37-39, wherein the nucleotide sequence encodes a human recombinant AAT polypeptide sequence of at least one of: SEQ ID NO:1, SEQ ID NO:3, SEQ ID NO:5, and SEQ ID NO:6.

Specific embodiment 41) The expression vector according to any of specific embodiments 37-40, wherein the nucleotide sequence encoding a human recombinant AAT polypeptide sequence comprises a sequence of at least one of: SEQ ID NO:2, SEQ ID NO:4, SEQ ID NO:6, and SEQ ID NO:8.

Specific embodiment 42) The expression vector according to any of specific embodiments 37-41, wherein the expression vector comprises SEQ ID NO:9.

Specific embodiment 43) A human recombinant AAT protein, comprising a polypeptide sequence having about 99% identity to SEQ ID NO: 1.

Specific embodiment 44) The human recombinant AAT protein according to specific embodiment 43, comprising a polypeptide sequence having a SEQ ID NO:1 mutation, wherein the mutation is: a phenylalanine to a leucine at position 51 (F51L), a methionine to valine mutation at position 351 (M351V), a methionine to valine mutation at position 358 (M358V), or any combinations thereof.

Specific embodiment 45) The human recombinant AAT protein according to any of specific embodiments 43-44, comprising a sialic acid content ranging from about 3 moles of sialic acid per mole of AAT to about 12 moles of sialic acid per mole of AAT.

Specific embodiment 46) The recombinant AAT protein according to specific embodiment 45, where the sialic acid content ranges from about 4 moles of sialic acid per mole of AAT to about 6 moles of sialic acid per mole of AAT.

Specific embodiment 47) The recombinant AAT protein according to any of specific embodiments 45-46, wherein the sialic acid content exceeds that of a plasma-derived AAT protein by at least 10%.

Specific embodiment 48) A composition, comprising a human recombinant AAT protein produced by the method of any one of specific embodiments 1-36, and a pharmaceutically acceptable carrier.

Specific embodiment 49) A composition, comprising a human recombinant AAT protein according to any one of specific embodiments 43-47, and a pharmaceutically acceptable carrier.

Specific embodiment 50) A method of treating a subject suffering from an alpha 1-antitrypsin deficiency, comprising administering to the subject an effective amount of a human recombinant AAT protein according to any of specific embodiments 43-47 to ameliorate the alpha 1-antitrypsin deficiency in the subject, thereby treating the subject.

Specific embodiment 51) A method of treating a subject suffering from a disease that results in protease-induced tissue damage, comprising administering to the subject an effective amount of a human recombinant AAT protein according to any of specific embodiments 43-47 to ameliorate the protease-induced tissue damage in the subject, thereby treating the subject.

Specific embodiment 52) The method according to any of specific embodiments 50-51, wherein the human recombinant AAT protein is a pharmaceutical composition comprising a human recombinant AAT protein and a pharmaceutically acceptable carrier.

Specific embodiment 53) The method according to any of specific embodiments 50-52, wherein the administering occurs by at least one route selected from the group consisting of: intravenously, parenterally, intramucosally, topically, transdermally, and by inhalation.

Specific embodiment 54) A method for producing a human recombinant alpha 1-antitrypsin (AAT) protein, comprising: culturing a host cell with a first nucleic acid sequence encoding a human AAT protein and at least a second nucleic acid sequence encoding a transposase, wherein the culturing step occurs at a first period of time at a first temperature and at a second period of time at a second temperature, and optionally at a third period of time at a third temperature.

Specific embodiment 55) The method of specific embodiment 54, wherein the second temperature is less than the first temperature.

Specific embodiment 56) The method of specific embodiment 55, wherein the third temperature is less than the second temperature.

Specific embodiment 57) The method of specific embodiment 56, wherein the first temperature ranges from about 31° C. to about 37° C.

Specific embodiment 58) The method of specific embodiment 57, wherein the second temperature ranges from about 31° C. to about 37° C.

Specific embodiment 59) The method of specific embodiment 58, wherein the third temperature ranges from about 31° C. to about 37° C.

Specific embodiment 60) The method of specific embodiment 59, wherein the first period of time ranges from about 1-20 days.

Specific embodiment 61) The method of specific embodiment 60, wherein the second period of time ranges from about 1-20 days.

Specific embodiment 62) The method of specific embodiment 61, wherein the third period of time ranges from about 1-20 days.

Specific embodiment 63) The method of specific embodiment 62, wherein the culturing step further comprises adding a first feed and a second feed.

Specific embodiment 64) The method of specific embodiment 63, wherein the adding step occurs every other day.

Specific embodiment 65) The method of specific embodiment 64, wherein the adding step occurs every day.

Specific embodiment 66) The method of the specific embodiment 65, wherein the culture for production is oxygenated with air only under avoidance of pure oxygen.

Specific embodiment 67) A method of treating a subject suffering from an overproduction of immune factors, comprising administering to the subject an effective amount of a human recombinant AAT protein according to any one of specific embodiments 43-47 to reduce overproduction of immune factors, thereby treating the subject.

Specific embodiment 68) The method of specific embodiment 67, wherein the immune factor is selected from TNF, IL-2, or the like, or any combinations thereof.

As various changes can be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings. Accordingly, the present description is intended to embrace all such alternatives, modifications and variances that fall within the scope of the appended claims.

1)-68) (canceled)

69) A method for producing a human recombinant alpha 1-antitrypsin (AAT) protein, comprising:
a) introducing into a eukaryotic host cell, a first nucleic acid sequence encoding a human AAT protein and at least an additional nucleic acid sequence encoding a transposase;
b) culturing the eukaryotic host cell under conditions which allow expression of the first nucleic acid sequence encoding a human AAT protein;
c) selecting the eukaryotic host cell with the nucleic acid fragment expressing a human AAT protein, wherein the selected cells are clonally-derived cells expressing human recombinant AAT protein; and
d) isolating the human recombinant AAT protein from the clonally-derived cells, thereby producing the human recombinant AAT protein.

70) The method according to claim 69, wherein the transposase is a piggyBac transposase.

71) The method according to claim 69, wherein the eukaryotic host cell is a Chinese hamster ovary (CHO) cell line.

72) The method according to claim 69, wherein the eukaryotic host cell is transformed with the nucleic acid sequence encoding a human recombinant AAT protein.

73) The method according to claim 69, wherein the selecting step comprises:
a) culturing the clonally-derived cells expressing human recombinant AAT in a culture medium;
b) feeding the clonally-derived cells expressing human recombinant AAT with at least one feed;
c) maintaining the culture medium at a cell culture temperature;
d) decreasing the cell culture temperature; and
e) culturing the clonally-derived cells at the decreased cell culture temperature, wherein the clonally-derived cells express the human recombinant AAT protein at a titer of about 1 g/L or greater.

74) The method according to claim 73, wherein the cell culture temperature ranges from about 35° C. to about 38° C.

75) The method according to claim 73, wherein the decreased cell culture temperature ranges from about 25° C. to about 34° C.

76) The method according to claim 73, wherein the at least one feed comprises a neutral feed.

77) The method according to claim 76, wherein the neutral feed is in a volume ranging from about 1% to about 8% of the total cell culture volume.

78) The method according to claim 73, wherein the at least one feed comprises an alkaline feed.

79) The method according to claim 78, wherein the alkaline feed is in a volume ranging from about 0.1% to about 0.8% of the total cell culture volume.

80) The method according to claim 69, wherein the culturing step comprises an osmolarity of the cell culture of about 550 mOsm/kg or greater.

81) The method according to claim 69, wherein the isolated human recombinant protein has a purity of about or greater than about 95%.

82) An expression vector, comprising: a nucleic acid fragment containing a nucleotide sequence encoding a human recombinant AAT protein, wherein the nucleic acid fragment is positioned in a multiple cloning site; an intron upstream of the nucleic acid fragment; a cytomegalovirus (CMV) promoter upstream of an intron; a 5′ Inverted Terminal Repeat (5′ ITR) upstream of the CMV promoter; a poly-adenosine tail signal sequence downstream of the nucleic acid fragment; a replication origin sequence downstream of the nucleic acid fragment; a selectable marker sequence downstream of the replication origin sequence; and a 3′ Inverted Terminal Repeat (3′ ITR) downstream of the selectable marker sequence.

83) The expression vector according to claim 83, wherein the selectable marker sequence is a puromycin resistance gene.

84) The expression vector according to claim 82, wherein the nucleic acid fragment and the selectable marker sequence are positioned in opposite reading frames and in between the 5′ ITR and the 3′ ITR.

85) The expression vector according to claim 82, wherein the nucleotide sequence encodes a human recombinant AAT polypeptide sequence of at least one of: SEQ ID NO:1, SEQ ID NO:3, SEQ ID NO:5, and SEQ ID NO:6.

86) The expression vector according to claim 82, wherein the nucleotide sequence encoding a human recombinant AAT polypeptide sequence comprises a sequence of at least one of: SEQ ID NO:2, SEQ ID NO:4, SEQ ID NO:6, and SEQ ID NO:8.

87) The expression vector according to claim 82, wherein the expression vector comprises SEQ ID NO:9.

88) A method for producing a human recombinant alpha 1-antitrypsin (AAT) protein, comprising: culturing a host cell with a first nucleic acid sequence encoding a human AAT protein and at least a second nucleic acid sequence encoding a transposase, wherein the culturing step occurs at a first period of time at a first temperature and at a second period of time at a second temperature, and optionally at a third period of time at a third temperature.


SEQUENCE LISTING (18 sequences)

SEQ ID NO: 1 - protein, 394 aa, Homo sapiens
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 2 - DNA, 1210 bp, Homo sapiens
  misc_feature (1)..(6): Restriction Enzyme Recognition Sequence
  misc_feature (1205)..(1210): Restriction Enzyme Recognition Sequence
actagtcaccgaagatcctcaaggtgacgccgcccaaaagaccgatacctcgcatcatgaccaagaccacccgacctttaacaagatcactccaaacctggccgagttcgcattctccctctacagacagctggctcaccagtcaaactcaaccaacatcttcttctcccctgtgagcatcgccactgcgttcgccatgctttcactgggcaccaaagccgatacgcacgacgagatcctggaggggctcaactttaaccttaccgaaatcccggaagcgcaaatccacgaaggattccaagaacttctgcgcaccctcaatcagccagactcgcagttgcagctgactaccggcaacggactgtttctctcggaagggctgaaactcgtggacaaattcctcgaggacgtgaagaagctgtaccattcggaggcgtttaccgtcaatttcggagataccgaagaagctaaaaagcaaatcaatgactacgtggagaagggaacccagggaaagatcgtggacctcgtcaaggaattggaccgggacaccgtgttcgccctggtgaattacatcttctttaaaggaaagtgggaaagaccattcgaggtgaaggatactgaggaagaagatttccacgtcgatcaggtgactaccgtgaaggtccccatgatgaagcgcctgggcatgttcaacatccagcactgtaagaagctgtcctcgtgggtcctgctcatgaagtacctgggaaatgcaactgctattttcttcctcccggatgagggcaaactgcagcaccttgagaacgagctgactcatgatatcattacgaagtttctggaaaatgaggacaggcggagcgccagcctccatctcccaaagctgtccatcacggggacgtatgacctgaagtcagtccttggacagctgggcatcactaaggtgtttagcaacggtgctgacttgtccggagtgactgaagaggcaccgctgaaactgtctaaggcggtccacaaggccgtgctcaccatcgacgaaaagggaactgaggccgctggagcaatgttcttggaggcgatcccgatgtcgatccctcccgaagtgaagttcaataagccgttcgtgtttctgatgattgagcaaaacactaaaagccctctgttcatgggtaaagtggtgaacccgactcagaagtagtgatgataagaattc

SEQ ID NO: 3 - protein, 418 aa, Homo sapiens
  SIGNAL (1)..(24): Natural human AAT leader sequence
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 4 - DNA, 1282 bp, Homo sapiens
  misc_feature (1)..(6): Restriction Enzyme Recognition Site
  misc_feature (1277)..(1282): Restriction Enzyme Recognition Site
actagtcaccatgccgagcagcgtgagctggggcattctgctgctggcgggcctgtgctgcctggtgccggtgagcctggcggaagatcctcaaggtgacgccgcccaaaagaccgatacctcgcatcatgaccaagaccacccgacctttaacaagatcactccaaacctggccgagttcgcattctccctctacagacagctggctcaccagtcaaactcaaccaacatcttcttctcccctgtgagcatcgccactgcgttcgccatgctttcactgggcaccaaagccgatacgcacgacgagatcctggaggggctcaactttaaccttaccgaaatcccggaagcgcaaatccacgaaggattccaagaacttctgcgcaccctcaatcagccagactcgcagttgcagctgactaccggcaacggactgtttctctcggaagggctgaaactcgtggacaaattcctcgaggacgtgaagaagctgtaccattcggaggcgtttaccgtcaatttcggagataccgaagaagctaaaaagcaaatcaatgactacgtggagaagggaacccagggaaagatcgtggacctcgtcaaggaattggaccgggacaccgtgttcgccctggtgaattacatcttctttaaaggaaagtgggaaagaccattcgaggtgaaggatactgaggaagaagatttccacgtcgatcaggtgactaccgtgaaggtccccatgatgaagcgcctgggcatgttcaacatccagcactgtaagaagctgtcctcgtgggtcctgctcatgaagtacctgggaaatgcaactgctattttcttcctcccggatgagggcaaactgcagcaccttgagaacgagctgactcatgatatcattacgaagtttctggaaaatgaggacaggcggagcgccagcctccatctcccaaagctgtccatcacggggacgtatgacctgaagtcagtccttggacagctgggcatcactaaggtgtttagcaacggtgctgacttgtccggagtgactgaagaggcaccgctgaaactgtctaaggcggtccacaaggccgtgctcaccatcgacgaaaagggaactgaggccgctggagcaatgttcttggaggcgatcccgatgtcgatccctcccgaagtgaagttcaataagccgttcgtgtttctgatgattgagcaaaacactaaaagccctctgttcatgggtaaagtggtgaacccgactcagaagtagtgatgataagaattc

SEQ ID NO: 5 - protein, 413 aa, Homo sapiens
  SIGNAL (1)..(19): Human IgG heavy chain leader sequence
MEFWLSWVFLVAILKGVQCEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 6 - DNA, 1267 bp, Homo sapiens
  misc_feature (1)..(6): Restriction Enzyme Recognition Sequence
  misc_feature (1262)..(1267): Restriction Enzyme Recognition Sequence
actagtcaccatggaattttggctgtcctgggttttcctcgttgcaatcttgaaaggcgtccagtgcgaagatcctcaaggtgacgccgcccaaaagaccgatacctcgcatcatgaccaagaccacccgacctttaacaagatcactccaaacctggccgagttcgcattctccctctacagacagctggctcaccagtcaaactcaaccaacatcttcttctcccctgtgagcatcgccactgcgttcgccatgctttcactgggcaccaaagccgatacgcacgacgagatcctggaggggctcaactttaaccttaccgaaatcccggaagcgcaaatccacgaaggattccaagaacttctgcgcaccctcaatcagccagactcgcagttgcagctgactaccggcaacggactgtttctctcggaagggctgaaactcgtggacaaattcctcgaggacgtgaagaagctgtaccattcggaggcgtttaccgtcaatttcggagataccgaagaagctaaaaagcaaatcaatgactacgtggagaagggaacccagggaaagatcgtggacctcgtcaaggaattggaccgggacaccgtgttcgccctggtgaattacatcttctttaaaggaaagtgggaaagaccattcgaggtgaaggatactgaggaagaagatttccacgtcgatcaggtgactaccgtgaaggtccccatgatgaagcgcctgggcatgttcaacatccagcactgtaagaagctgtcctcgtgggtcctgctcatgaagtacctgggaaatgcaactgctattttcttcctcccggatgagggcaaactgcagcaccttgagaacgagctgactcatgatatcattacgaagtttctggaaaatgaggacaggcggagcgccagcctccatctcccaaagctgtccatcacggggacgtatgacctgaagtcagtccttggacagctgggcatcactaaggtgtttagcaacggtgctgacttgtccggagtgactgaagaggcaccgctgaaactgtctaaggcggtccacaaggccgtgctcaccatcgacgaaaagggaactgaggccgctggagcaatgttcttggaggcgatcccgatgtcgatccctcccgaagtgaagttcaataagccgttcgtgtttctgatgattgagcaaaacactaaaagccctctgttcatgggtaaagtggtgaacccgactcagaagtagtgatgataagaattc

SEQ ID NO: 7 - protein, 418 aa, Homo sapiens
  SIGNAL (1)..(24): Chimpanzee AAT leader sequence
MLSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 8 - DNA, 1282 bp, Homo sapiens
  misc_feature (1)..(6): Restriction Enzyme Recognition Sequence
  misc_feature (1277)..(1282): Restriction Enzyme Recognition Sequence
actagtcaccatgctgagcagcgtgagctggggcattctgctgctggcgggcctgtgctgcctggtgccggtgagcctggcggaagatcctcaaggtgacgccgcccaaaagaccgatacctcgcatcatgaccaagaccacccgacctttaacaagatcactccaaacctggccgagttcgcattctccctctacagacagctggctcaccagtcaaactcaaccaacatcttcttctcccctgtgagcatcgccactgcgttcgccatgctttcactgggcaccaaagccgatacgcacgacgagatcctggaggggctcaactttaaccttaccgaaatcccggaagcgcaaatccacgaaggattccaagaacttctgcgcaccctcaatcagccagactcgcagttgcagctgactaccggcaacggactgtttctctcggaagggctgaaactcgtggacaaattcctcgaggacgtgaagaagctgtaccattcggaggcgtttaccgtcaatttcggagataccgaagaagctaaaaagcaaatcaatgactacgtggagaagggaacccagggaaagatcgtggacctcgtcaaggaattggaccgggacaccgtgttcgccctggtgaattacatcttctttaaaggaaagtgggaaagaccattcgaggtgaaggatactgaggaagaagatttccacgtcgatcaggtgactaccgtgaaggtccccatgatgaagcgcctgggcatgttcaacatccagcactgtaagaagctgtcctcgtgggtcctgctcatgaagtacctgggaaatgcaactgctattttcttcctcccggatgagggcaaactgcagcaccttgagaacgagctgactcatgatatcattacgaagtttctggaaaatgaggacaggcggagcgccagcctccatctcccaaagctgtccatcacggggacgtatgacctgaagtcagtccttggacagctgggcatcactaaggtgtttagcaacggtgctgacttgtccggagtgactgaagaggcaccgctgaaactgtctaaggcggtccacaaggccgtgctcaccatcgacgaaaagggaactgaggccgctggagcaatgttcttggaggcgatcccgatgtcgatccctcccgaagtgaagttcaataagccgttcgtgtttctgatgattgagcaaaacactaaaagccctctgttcatgggtaaagtggtgaacccgactcagaagtagtgatgataagaattc

SEQ ID NO: 9 - DNA, 6504 bp, Homo sapiens
  misc_feature (1948)..(1953): Restriction Enzyme Recognition Sequence
  misc_feature (2015)..(3196): CHO-cell codon-optimized AAT protein sequence
  misc_feature (3209)..(3214): Restriction Enzyme Recognition Sequence
ggcgcgccttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcgataacaggaaagttccattggagccaagtacattgagtcaatagggactttccaatgggttttgcccagtacataaggtcaatgggaggtaagccaatgggtttttcccattactggcacgtatactgagtcattagggactttccaatgggttttgcccagtacataaggtcaataggggtgaatcaacaggaaagttccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtagaacgcagagctcctcgctgcaggcaagcttggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcagggatccactagtcaccatggaattttggctgtcctgggttttcctcgttgcaatcttgaaaggcgtccagtgcgaagatcctcaaggtgacgccgcccaaaagaccgatacctcgcatcatgaccaagaccacccgacctttaacaagatcactccaaacctggccgagttcgcattctccctctacagacagctggctcaccagtcaaactcaaccaacatcttcttctcccctgtgagcatcgccactgcgttcgccatgctttcactgggcaccaaagccgatacgcacgacgagatcctggaggggctcaactttaaccttaccgaaatcccggaagcgcaaatccacgaaggattccaagaacttctgcgcaccctcaatcagccagactcgcagttgcagctgactaccggcaacggactgtttctctcggaagggctgaaactcgtggacaaattcctcgaggacgtgaagaagctgtaccattcggaggcgtttaccgtcaatttcggagataccgaagaagctaaaaagcaaatcaatgactacgtggagaagggaacccagggaaagatcgtggacctcgtcaaggaattggaccgggacaccgtgttcgccctggtgaattacatcttctttaaaggaaagtgggaaagaccattcgaggtgaaggatactgaggaagaagatttccacgtcgatcaggtgactaccgtgaaggtccccatgatgaagcgcctgggcatgttcaacatccagcactgtaagaagctgtcctcgtgggtcctgctcatgaagtacctgggaaatgcaactgctattttcttcctcccggatgagggcaaactgcagcaccttgagaacgagctgactcatgatatcattacgaagtttctggaaaatgaggacaggcggagcgccagcctccatctcccaaagctgtccatcacggggacgtatgacctgaagtcagtccttggacagctgggcatcactaaggtgtttagcaacggtgctgacttgtccggagtgactgaagaggcaccgctgaaactgtctaaggcggtccacaaggccgtgctcaccatcgacgaaaagggaactgaggccgctggagcaatgttcttggaggcgatcccgatgtcgatccctcccgaagtgaagttcaataagccgttcgtgtttctgatgattgagcaaaacactaaaagccctctgttcatgggtaaagtggtgaacccgactcagaagtagtgatgataagaattctgcagatatccatcacactggcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagtggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcataacttgtttattgcagcttataatggttacaaataaagcaatagcatcacaaatttcacaaataaagcatttttttcactgcattctagttgtggtttgtccaaactcatcaatgtatcttatcatgtctggatccgcttcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagcctggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggtggcctgcagagtcgctctgtgttcgaggccacacgcgtcaccttaatatgcgaagtggacctgggaccgcgccgccccgactgcatctgcgtgttttcgccaatgacaagacgctgggcggggtttgtgtcatcatagaactaaagacatgcaaatatatttcttccggggacaccgccagcaaacgcgagcaacgggccacggggatgaagcagctggctagctaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaattcgaacagctggttctttccgcctcaggactcttcctttttcaataaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaat

SEQ ID NO: 10 - protein, 594 aa, Trichoplusia ni
MGSSLDDEHILSALLQSDDELVGEDSDSEISDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 11 - protein, 24 aa, Homo sapiens
  SIGNAL (1)..(24): H. sapiens & Hylobates sp. natural AAT leader sequence
MPSSVSWGILLLAGLCCLVPVSLA

SEQ ID NO: 12 - protein, 19 aa, Homo sapiens
  Signal (1)..(19): H. sapiens IgG Heavy chain leader sequence
MEFWLSWVFLVAILKGVQC

SEQ ID NO: 13 - protein, 24 aa, Pan troglodytes
  Signal (1)..(24): Pan troglodytes (Chimpanzee) AAT leader sequence
MLSSVSWGILLLAGLCCLVPVSLA

SEQ ID NO: 14 - protein, 18 aa, Homo sapiens
  Signal (1)..(18): H. sapiens serum albumin leader sequence
MKWVTFISLLFLFSSAYS

SEQ ID NO: 15 - protein, 19 aa, Homo sapiens
  Signal (1)..(19): H. sapiens Azurocidin leader sequence
MTRLTVLALLAGLLASSRA

SEQ ID NO: 16 - protein, 22 aa, Homo sapiens
  Signal (1)..(22): H. sapiens Ig kappa Light chain leader sequence
METPAQLLFLLLLWLPVSDTTG

SEQ ID NO: 17 - protein, 20 aa, Mus musculus
  Signal (1)..(20): Mouse and Hamster Ig kappa Light chain
METDTLLLWVLLLWVPGSTG

SEQ ID NO: 18 - protein, 9 aa, Artificial Sequence
  Synthetic fluorogenic peptide
  MISC_FEATURE (1)..(1): (7-Methoxycoumarin-4-yl)acetyl (Mca) attached before residue position 1
  MOD_RES (6)..(6): Xaa = Norvaline (Nva)
  MISC_FEATURE (9)..(9): 2,4-Dinitrophenyl (Dnp) attached after residue position 9
RPKVEXWRK